(19) United States
(12) Patent Application Publication
(10) Pub. No.: US 2021/0009557 A1
(43) Pub. Date: Jan. 14, 2021

(54) ACYCLIC CXCR4 INHIBITORS AND USES THEREOF

(71) Applicant: X4 Pharmaceuticals, Inc., Cambridge, MA (US)

(72) Inventors: Elyse Marie Josee Bourque, L'Etang-du-Nord (CA); Renato Skerlj, West Newton, MA (US)

(21) Appl. No.: 16/955,205

(22) PCT Filed: Dec. 18, 2018
(86) PCT No.: PCT/US18/66141

§ 371 Date: Jun. 18, 2020

Related U.S. Application Data
(60) Provisional application No. 62/607,623, filed on Dec. 19, 2017.

(51) Int. Cl. C07D 401/14; C07D 401/12; C07D 213/36; C07D 413/14

(52) U.S. Cl. CPC C07D 401/14; C07D 413/14; C07D 213/36; C07D 401/12

(57) ABSTRACT

The present invention relates to compounds and methods useful for modulation, e.g. inhibition, of C-X-C receptor type 4 (CXCR4). The invention also provides pharmaceutically acceptable compositions comprising compounds of the present invention and methods of using the compositions in the treatment of various disorders.

### TECHNICAL FIELD OF THE INVENTION

The present invention relates to compounds and methods useful for modulation, e.g. inhibition, of C-X-C receptor type 4 (CXCR4). The invention also provides pharmaceutically acceptable compositions comprising compounds of the present invention and methods of using said compositions in the treatment of various disorders, such as certain cancers.

### CROSS-REFERENCE TO RELATED APPLICATIONS

This application claims the benefit of U.S. Provisional Patent Application No. 62/607,623, filed Dec. 19, 2017, the entire contents of which is hereby incorporated by reference.

### BACKGROUND OF THE INVENTION

C-X-C chemokine receptor type 4 (CXCR4), also known as fusin or cluster of differentiation 184 (CD184), is a seven transmembrane G-protein coupled receptor (GPCR) belonging to Class I GPCR or rhodopsin-like GPCR family. Under normal physiological conditions, CXCR4 carries out multiple roles and is principally expressed in the hematopoietic and immune systems. CXCR4 was initially discovered as one of the co-receptors involved in human immunodeficiency virus (HIV) cell entry. Subsequent studies showed that it is expressed in many tissues, including brain, thymus, lymphatic tissues, spleen, stomach, and small intestine, and also specific cell types such as hematopoietic stem cells (HSC), mature lymphocytes, and fibroblasts. CXCL12, previously designated SDF-1α, is the only known ligand for CXCR4. CXCR4 mediates migration of stem cells during embryonic development as well as in response to injury and inflammation. Multiple roles have been demonstrated for CXCR4 in human diseases such as cellular proliferative disorders, Alzheimer's disease, HIV, rheumatoid arthritis, pulmonary fibrosis, and others. For example, expression of CXCR4 and CXCL12 have been noted in several tumor types. CXCL12 is expressed by cancer-associated fibroblast (CAFs) and is often present at high levels in the tumor microenvironment (TME). In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver, and brain, which are sites of CXCL12 expression. CXCR4 is frequently expressed on melanoma cells, particularly the CD133+ population that is considered to represent melanoma stem cells; in vitro experiments and murine models have demonstrated that CXCL12 is chemotactic for such cells.

Furthermore, there is now evidence implicating the CXCL12/CXCR4 axis in contributing to the loss or lack of tumor responsiveness to angiogenesis inhibitors (also referred to as “angiogenic escape”). In animal cancer models, interference with CXCR4 function has been demonstrated to alter the TME and sensitize the tumor to immune attack by multiple mechanisms such as elimination of tumor re-vascularization and increasing the ratio of CD8⁺ T cells to Treg cells. These effects result in significantly decreased tumor burden and increased overall survival in xenograft, syngeneic, and transgenic cancer models. See Vanharanta et al. (2013) Nat Med 19: 50-56; Gale and McColl (1999) BioEssays 21: 17-28; Highfill et al. (2014) Sci Transl Med 6: ra67; Facciabene et al. (2011) Nature 475: 226-230.

These data underscore the significant, unmet need for CXCR4 inhibitors to treat the many diseases and conditions mediated by aberrant or undesired expression of the receptor, for example in cellular proliferative disorders.

### SUMMARY OF THE INVENTION

It has now been found that compounds of the present invention, and pharmaceutically acceptable compositions thereof, are effective as CXCR4 inhibitors. In one aspect, the present invention provides a compound of Formula I:

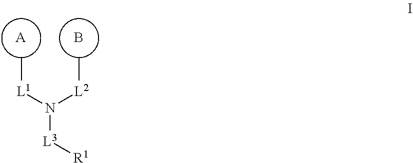

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with CXC receptor type 4 (CXCR4). Such diseases, disorders, or conditions include cellular proliferative disorders (e.g., cancer) such as those described herein.

### DETAILED DESCRIPTION OF CERTAIN EMBODIMENTS

### 1. General Description of Certain Embodiments of the Invention

Compounds of the present invention, and pharmaceutical compositions thereof, are useful as inhibitors of CXCR4. Without wishing to be bound by any particular theory, it is believed that compounds of the present invention, and pharmaceutical compositions thereof, may inhibit the activity of CXCR4 and thus treat certain diseases, such as cancer.

It has now been found that compounds of this invention, and pharmaceutically acceptable compositions thereof, are effective as CXCR4 inhibitors. In one aspect, the present invention provides a compound of Formula I:

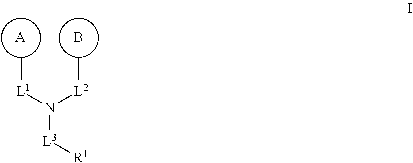

or a pharmaceutically acceptable salt thereof, wherein:
Ring A is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
Ring B is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
L¹ is —CH₂— or —CH(CH₃)—;
L² is a covalent bond, —CH₂—, or —CH(CH₃)—;
L³ is a C₂₋₃ bivalent straight or branched hydrocarbon chain;
R¹ is -Cy, —OR, —N(R)₂, —C(O)N(R)₂, or —N(R)C(O)R;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur,
   or two R groups on the same nitrogen are optionally take together with their intervening atoms to form a 5-6 membered saturated, partially unsaturated, or aromatic heterocyclic ring having 1-2 heteroatoms in addition to the nitrogen attached thereto independently selected from nitrogen, oxygen, or sulfur; and
-Cy is an optionally substituted ring selected from a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

In some embodiments, the present invention provides a compound of Formula I wherein said compound is other than a compound selected from:

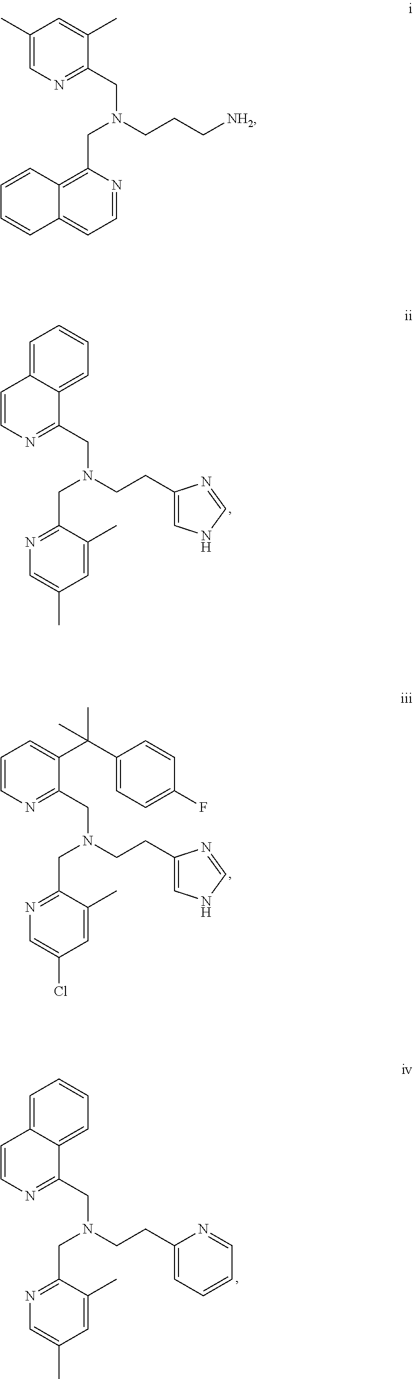 [SMILES: Cc1cnc(CN(CCCN)Cc2nccc3ccccc23)c(C)c1 ; Cc1cnc(CN(CCc2c[nH]cn2)Cc2nccc3ccccc23)c(C)c1 ; Cc1cc(Cl)cnc1CN(CCc1c[nH]cn1)Cc1ncccc1C(C)(C)c1ccc(F)cc1 ; Cc1cnc(CN(CCc2ccccn2)Cc2nccc3ccccc23)c(C)c1]

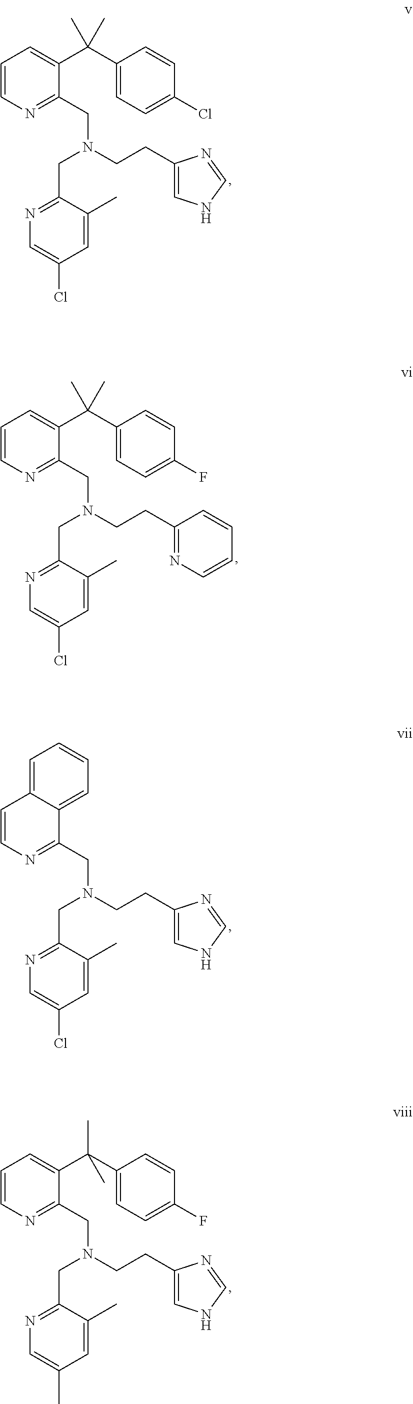 [SMILES: Cc1cc(Cl)cnc1CN(CCc1c[nH]cn1)Cc1ncccc1C(C)(C)c1ccc(Cl)cc1 ; Cc1cc(Cl)cnc1CN(CCc1ccccn1)Cc1ncccc1C(C)(C)c1ccc(F)cc1 ; Cc1cc(Cl)cnc1CN(CCc1c[nH]cn1)Cc1nccc2ccccc12 ; Cc1cnc(CN(CCc2c[nH]cn2)Cc2ncccc2C(C)(C)c2ccc(F)cc2)c(C)c1]

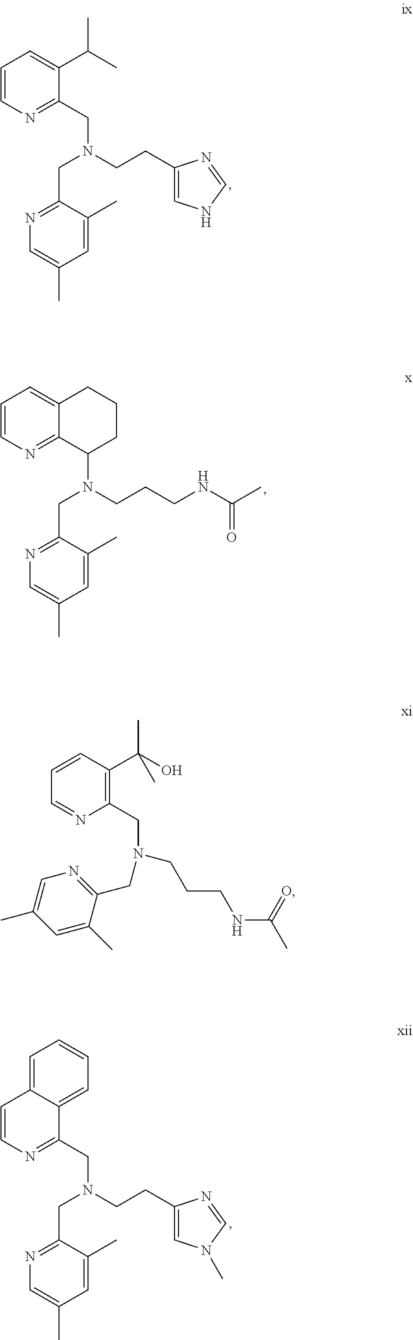

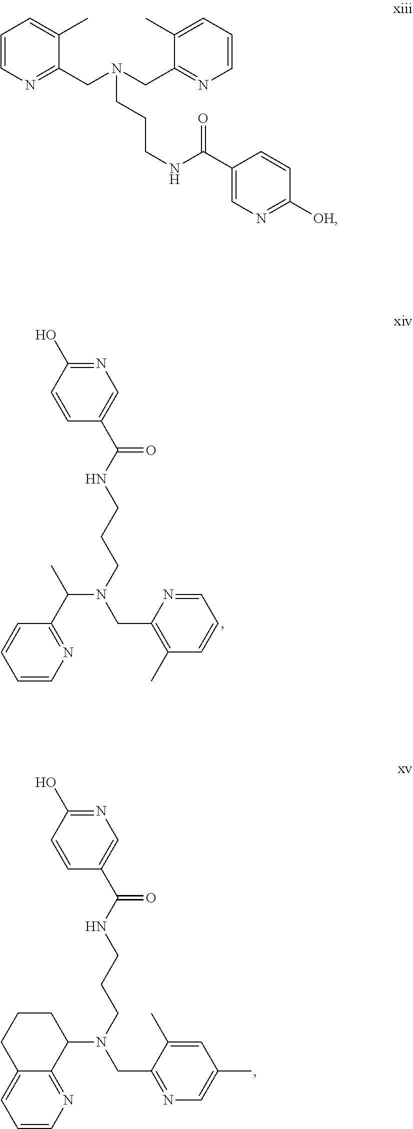 [SMILES: Cc1cccnc1CN(CCCNC(=O)c1ccc(O)nc1)Cc1ncccc1C ; Cc1cccnc1CN(CCCNC(=O)c1ccc(O)nc1)C(C)c1ccccn1 ; Cc1cnc(CN(CCCNC(=O)c2ccc(O)nc2)C2CCCc3cccnc32)c(C)c1]

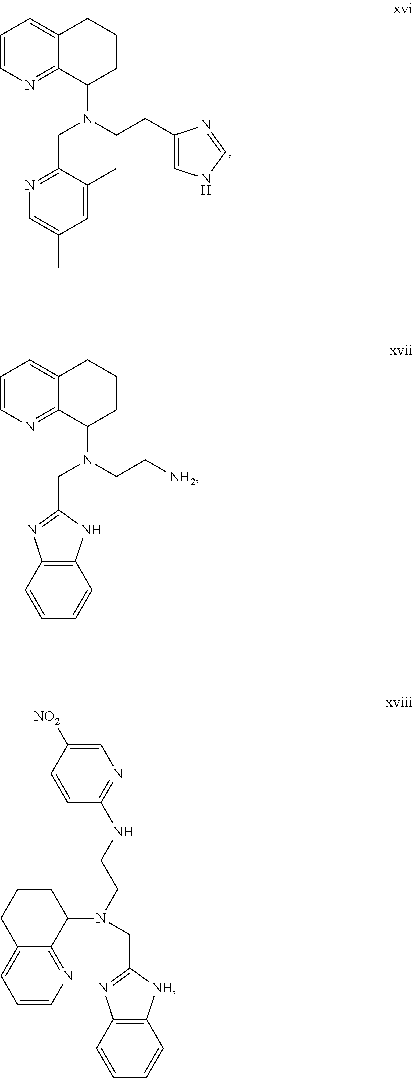 [SMILES: Cc1cnc(CN(CCc2c[nH]cn2)C2CCCc3cccnc32)c(C)c1 ; NCCN(Cc1nc2ccccc2[nH]1)C1CCCc2cccnc21 ; O=[N+]([O-])c1ccc(NCCN(Cc2nc3ccccc3[nH]2)C2CCCc3cccnc32)nc1]

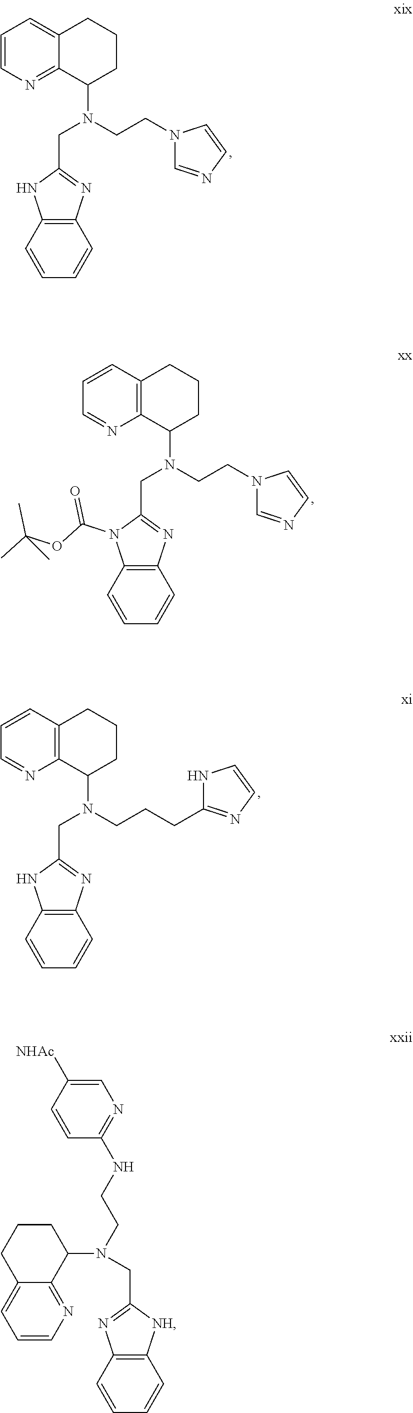 [SMILES: c1cnc2c(c1)CCCC2N(CCn1ccnc1)Cc1nc2ccccc2[nH]1 ; CC(C)(C)OC(=O)n1c(CN(CCn2ccnc2)C2CCCc3cccnc32)nc2ccccc21 ; c1cnc2c(c1)CCCC2N(CCCc1ncc[nH]1)Cc1nc2ccccc2[nH]1 ; CC(=O)Nc1ccc(NCCN(Cc2nc3ccccc3[nH]2)C2CCCc3cccnc32)nc1]

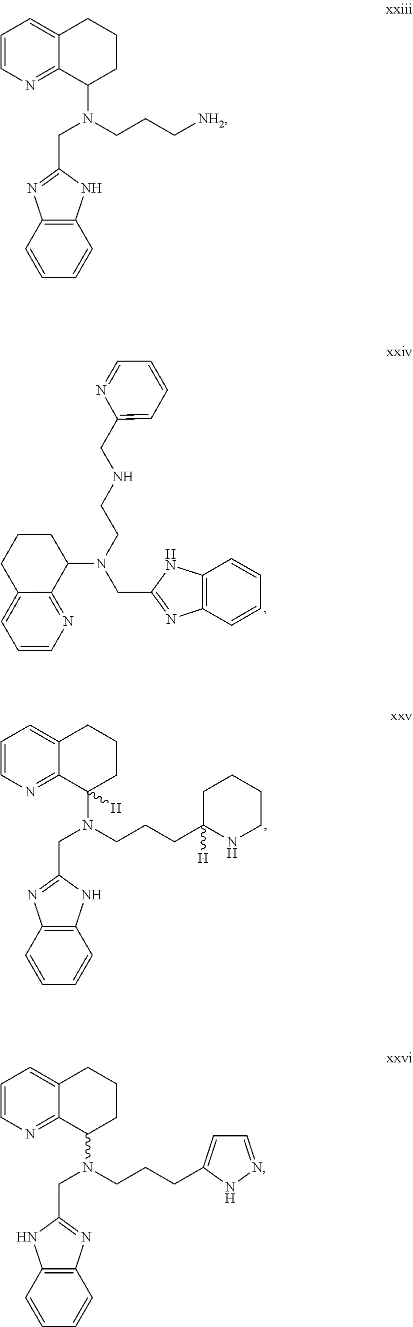 [SMILES: NCCCN(Cc1nc2ccccc2[nH]1)C1CCCc2cccnc21 ; c1ccc(CNCCN(Cc2nc3ccccc3[nH]2)C2CCCc3cccnc32)nc1 ; c1cnc2c(c1)CCCC2N(CCCC1CCCCN1)Cc1nc2ccccc2[nH]1 ; c1cnc2c(c1)CCCC2N(CCCc1ccn[nH]1)Cc1nc2ccccc2[nH]1]

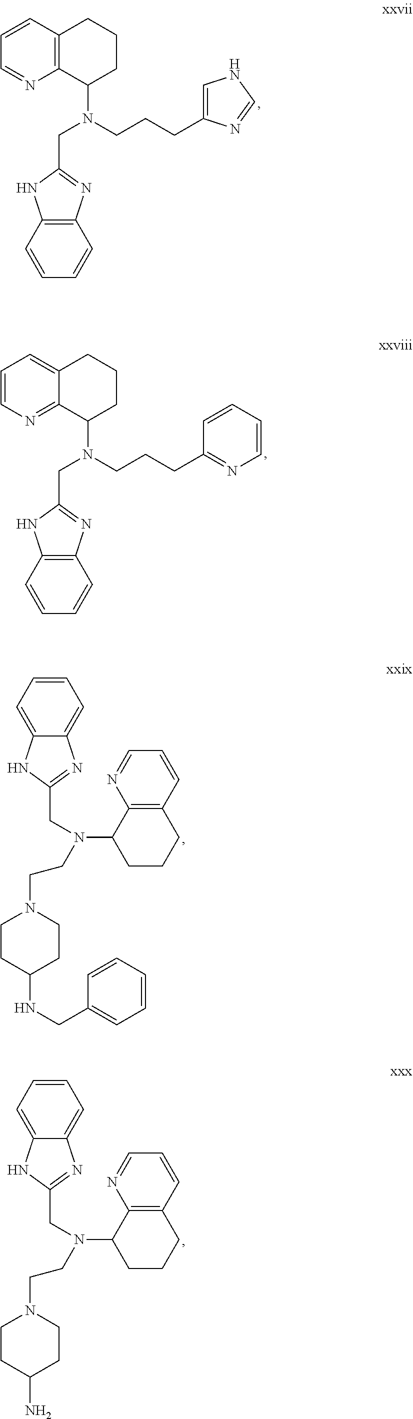 [SMILES: c1cnc2c(c1)CCCC2N(CCCc1c[nH]cn1)Cc1nc2ccccc2[nH]1 ; c1ccc(CCCN(Cc2nc3ccccc3[nH]2)C2CCCc3cccnc32)nc1 ; c1ccc(CNC2CCN(CCN(Cc3nc4ccccc4[nH]3)C3CCCc4cccnc43)CC2)cc1 ; NC1CCN(CCN(Cc2nc3ccccc3[nH]2)C2CCCc3cccnc32)CC1]

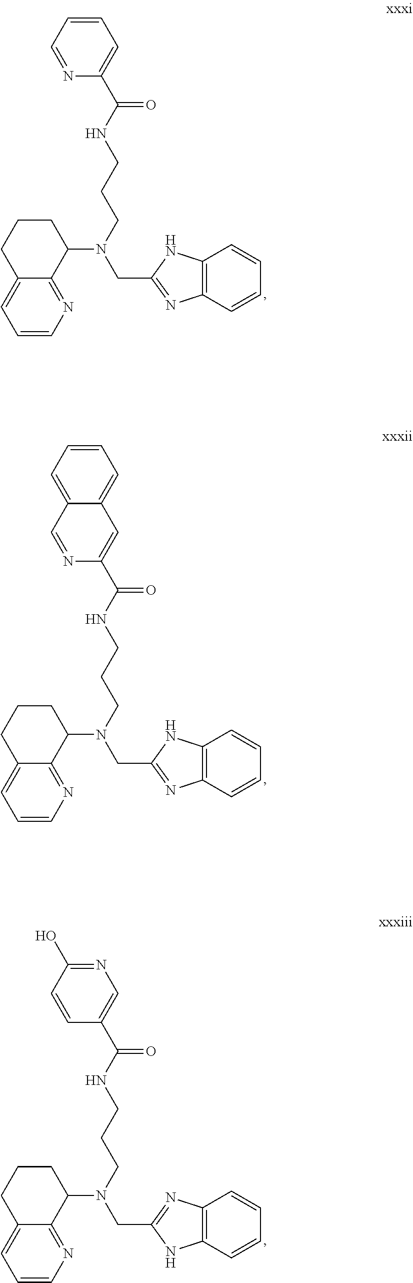 [SMILES: O=C(NCCCN(Cc1nc2ccccc2[nH]1)C1CCCc2cccnc21)c1ccccn1 ; O=C(NCCCN(Cc1nc2ccccc2[nH]1)C1CCCc2cccnc21)c1cc2ccccc2cn1 ; O=C(NCCCN(Cc1nc2ccccc2[nH]1)C1CCCc2cccnc21)c1ccc(O)nc1]

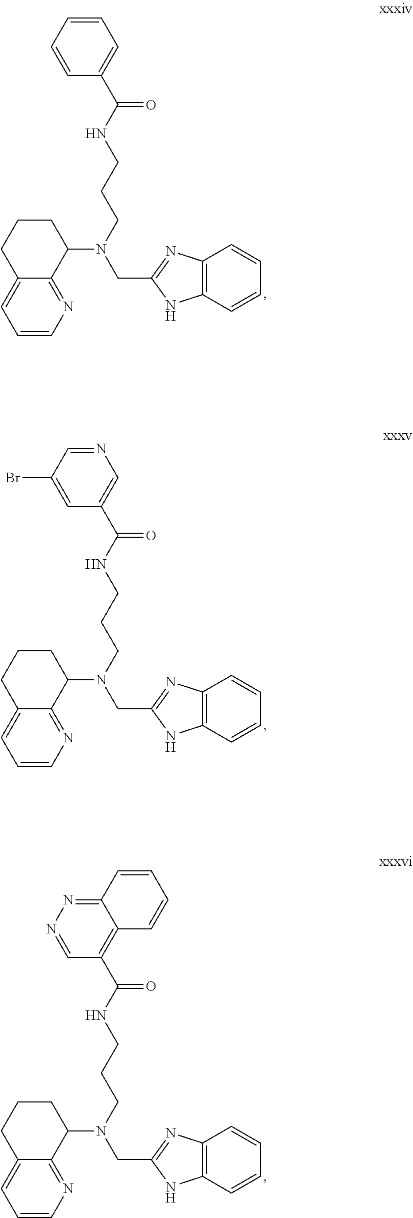 [SMILES: O=C(NCCCN(Cc1nc2ccccc2[nH]1)C1CCCc2cccnc21)c1ccccc1 ; O=C(NCCCN(Cc1nc2ccccc2[nH]1)C1CCCc2cccnc21)c1cncc(Br)c1 ; O=C(NCCCN(Cc1nc2ccccc2[nH]1)C1CCCc2cccnc21)c1cnnc2ccccc12]

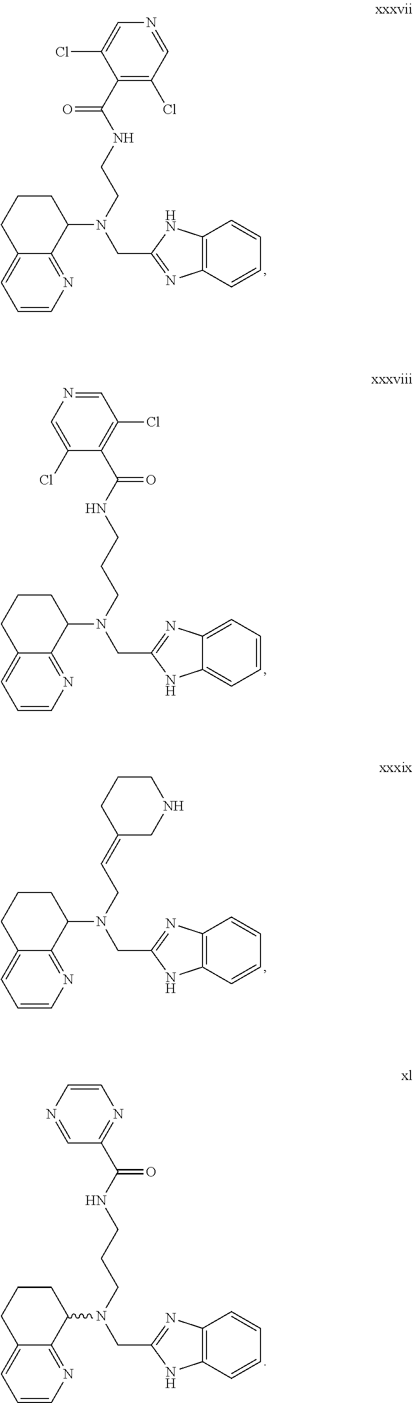 [SMILES: O=C(NCCN(Cc1nc2ccccc2[nH]1)C1CCCc2cccnc21)c1c(Cl)cncc1Cl ; O=C(NCCCN(Cc1nc2ccccc2[nH]1)C1CCCc2cccnc21)c1c(Cl)cncc1Cl ; C(/CN(Cc1nc2ccccc2[nH]1)C1CCCc2cccnc21)=C1\CCCNC1 ; O=C(NCCCN(Cc1nc2ccccc2[nH]1)C1CCCc2cccnc21)c1cnccn1]

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

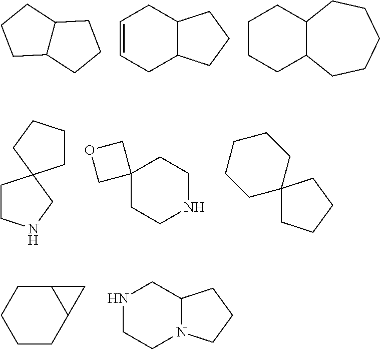

Exemplary bridged bicyclics include:

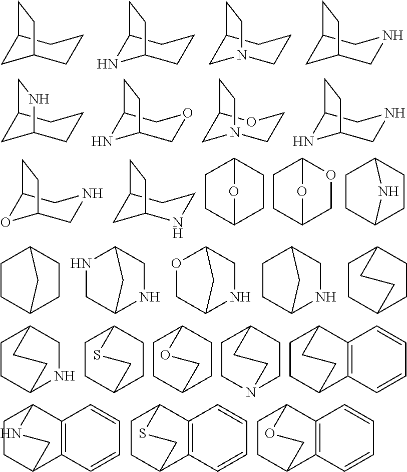

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

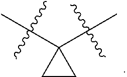 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the term “inhibitor” is defined as a compound that binds to and/or inhibits CXCR4 with measurable affinity. In certain embodiments, an inhibitor has an IC₅₀ and/or binding constant of less than about 100 μM, less than about 50 μM, less than about 1 μM, less than about 500 nM, less than about 100 nM, less than about 10 nM, or less than about 1 nM.

The terms “measurable affinity” and “measurably inhibit,” as used herein, means a measurable change in CXCR4 activity between a sample comprising a compound of the present invention, or composition thereof, and CXCR4, and an equivalent sample comprising CXCR4, in the absence of said compound, or composition thereof.

### 3. Description of Exemplary Embodiments

In some embodiments, the present invention provides a compound of Formula I

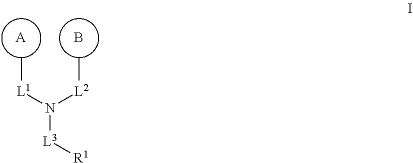

or a pharmaceutically acceptable salt thereof, wherein.
Ring A is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
Ring B is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
L¹ is —CH₂— or —CH(CH₃)—;
L² is a covalent bond, —CH₂—, or —CH(CH₃)—;
L³ is a C₂₋₃ bivalent straight or branched hydrocarbon chain;
R¹ is -Cy, —OR, —N(R)₂, —C(O)N(R)₂, or —N(R)C(O)R;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur,
   or two R groups on the same nitrogen are optionally take together with their intervening atoms to form a 5-6 membered saturated, partially unsaturated, or aromatic heterocyclic ring having 1-2 heteroatoms in addition to the nitrogen attached thereto independently selected from nitrogen, oxygen, or sulfur; and
-Cy is an optionally substituted ring selected from a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur.

As defined generally above, Ring A is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur. In some embodiments, Ring A is an optionally substituted ring selected from a 5-6 membered monocyclic heteroaromatic ring having 1-4 heteroatoms independently selected from nitrogen, oxygen, or sulfur. In certain embodiments, Ring A is an optionally substituted ring selected from a 5-membered monocyclic heteroaromatic ring having 1-4 heteroatoms independently selected from nitrogen, oxygen, or sulfur. In some embodiments, Ring A is an optionally substituted ring selected from a 5-membered monocyclic heteroaromatic ring having one nitrogen and one additional heteroatom selected from oxygen or sulfur. In some embodiments, Ring A is an optionally substituted ring selected from thiazolyl. In some embodiments, Ring A is an optionally substituted ring selected from a 6-membered monocyclic heteroaromatic ring having 1-2 nitrogens. In some embodiments, Ring A is an optionally substituted ring selected from pyridyl.

In other embodiments, Ring A is an optionally substituted ring selected from an 8-10 membered bicyclic partially unsaturated or aromatic ring having 0-3 heteroatoms independently selected from nitrogen, oxygen, or sulfur. In certain embodiments, Ring A is an optionally substituted ring selected from an 8-10 membered bicyclic aromatic ring having 1-3 heteroatoms independently selected from nitrogen, oxygen, or sulfur. In certain embodiments, Ring A is an optionally substituted ring selected from an 8-10 membered bicyclic aromatic ring having 1-2 nitrogens. In some embodiments, Ring A is an optionally substituted ring selected from benzimidazolyl or quinolinyl. In some embodiments, Ring A is an optionally substituted ring selected from an 8-10 membered bicyclic partially unsaturated or aromatic ring having 1-3 nitrogens. In certain embodiments, Ring A is an optionally substituted ring selected from tetrahydroquinolinyl.

In some embodiments, Ring A is selected from N or

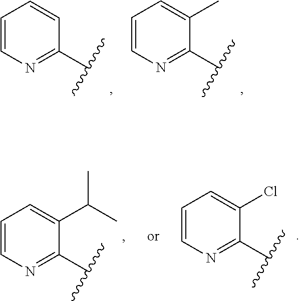

In some embodiments, Ring A is selected from

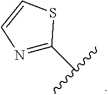 [SMILES: *c1nccs1]

In some embodiments, Ring A is selected from N or F

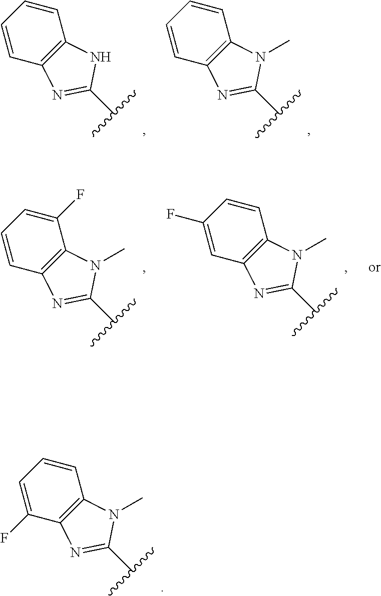 [SMILES: *c1nc2ccccc2[nH]1 ; *c1nc2ccccc2n1C ; *c1nc2cccc(F)c2n1C ; *c1nc2cc(F)ccc2n1C ; *c1nc2c(F)cccc2n1C]

In certain embodiments, Ring A is selected from those depicted in Table 1, below.

As defined generally above, Ring B is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur. In some embodiments, Ring B is an optionally substituted ring selected from a 5-6 membered monocyclic heteroaromatic ring having 1-4 heteroatoms independently selected from nitrogen, oxygen, or sulfur. In certain embodiments, Ring B is an optionally substituted ring selected from a 5-membered monocyclic heteroaromatic ring having 1-4 heteroatoms independently selected from nitrogen, oxygen, or sulfur. In some embodiments, Ring B is an optionally substituted ring selected from a 5-membered monocyclic heteroaromatic ring having one nitrogen and one additional heteroatom selected from oxygen or sulfur. In some embodiments, Ring B is an optionally substituted ring selected from thiazolyl. In some embodiments, Ring B is an optionally substituted ring selected from a 6-membered monocyclic heteroaromatic ring having 1-2 nitrogens. In some embodiments, Ring B is an optionally substituted ring selected from pyridyl.

In other embodiments, Ring B is an optionally substituted ring selected from an 8-10 membered bicyclic partially unsaturated or aromatic ring having 0-3 heteroatoms independently selected from nitrogen, oxygen, or sulfur. In certain embodiments, Ring B is an optionally substituted ring selected from an 8-10 membered bicyclic aromatic ring having 1-3 heteroatoms independently selected from nitrogen, oxygen, or sulfur. In certain embodiments, Ring B is an optionally substituted ring selected from an 8-10 membered bicyclic aromatic ring having 1-2 nitrogens. In some embodiments, Ring B is an optionally substituted ring selected from benzimidazolyl or quinolinyl. In some embodiments, Ring B is an optionally substituted ring selected from an 8-10 membered bicyclic partially unsaturated or aromatic ring having 1-3 nitrogens. In certain embodiments, Ring B is an optionally substituted ring selected from tetrahydroquinolinyl.

In some embodiments, Ring B is an optionally substituted ring selected from N

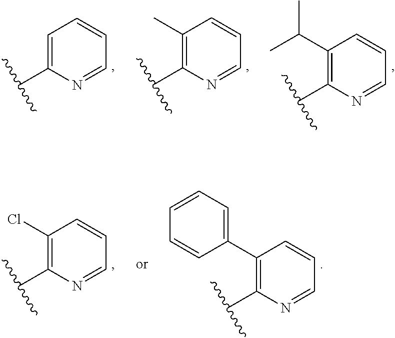

In some embodiments, Ring B is selected from

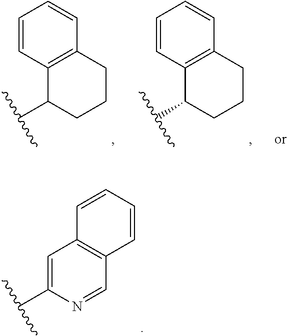 [SMILES: *C1CCCc2ccccc21 ; *[C@H]1CCCc2ccccc21 ; *c1cc2ccccc2cn1]

In certain embodiments, Ring B is selected from those depicted in Table 1, below.

As defined generally above, L¹ is —CH₂— or —CH(CH₃)—. In some embodiments, L¹ is —CH₂—. In some embodiments, L¹ is —CH(CH₃)—.

In certain embodiments, L¹ is selected from those depicted in Table 1, below.

As defined generally above, L² is a covalent bond, —CH₂—, or —CH(CH₃)—. In some embodiments, L² is a covalent bond. In other embodiments, L² is —CH₂— or —CH(CH₃)—. In some embodiments, L² is —CH₂—. In some embodiments, L² is —CH(CH₃)—.

In certain embodiments, L² is selected from those depicted in Table 1, below.

As defined generally above, L³ is a C₂₋₃ bivalent straight or branched hydrocarbon chain. In some embodiments, L³ is a C₂ bivalent straight or branched hydrocarbon chain. In some embodiments, L³ is a C₃ bivalent straight or branched hydrocarbon chain. In some embodiments, L³ is —CH₂CH₂—. In other embodiments, L³ is —CH₂CH₂CH₂—.

In certain embodiments, L³ is selected from those depicted in Table 1, below.

As defined generally above, R¹ is -Cy, —OR, —N(R)₂, —C(O)N(R)₂, or —N(R)C(O)R. In some embodiments, R¹ is —OR, —N(R)₂, —C(O)N(R)₂, or —N(R)C(O)R. In some embodiments, R¹ is —OR. In some embodiments, R¹ is —N(R)₂. In some embodiments, R¹ is —C(O)N(R)₂. In some embodiments, R¹ is —N(R)C(O)R. In some embodiments, R¹ is —OH, —N(H)₂, —C(O)N(H)₂, or —N(H)C(O)R. In some embodiments, R¹ is —OH or —OCH₃. In some embodiments, R¹ is —N(H)₂ or —NH(CH₃). In some embodiments, R¹ is —C(O)N(H)₂. In some embodiments, R¹ is —N(H)C(O)CH₃.

In certain embodiments, R¹ is -Cy. As defined generally above, -Cy is an optionally substituted ring selected from a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur. In certain embodiments, -Cy is an optionally substituted ring selected from a 3-8 membered saturated or partially unsaturated monocyclic carbocyclic ring. In certain embodiments, -Cy is optionally substituted phenyl. In certain embodiments, -Cy is an optionally substituted 8-10 membered bicyclic aromatic carbocyclic ring. In certain embodiments, -Cy is a 4-8 membered saturated or partially unsaturated monocyclic heterocyclic ring having 1-2 heteroatoms independently selected from nitrogen, oxygen, or sulfur. In certain embodiments, -Cy is an optionally substituted ring selected from a 5-6 membered monocyclic heteroaromatic ring having 1-4 heteroatoms independently selected from nitrogen, oxygen, or sulfur. In certain embodiments, -Cy is an optionally substituted ring selected from a 5-membered monocyclic heteroaromatic ring having 1-4 heteroatoms independently selected from nitrogen, oxygen, or sulfur. In some embodiments, -Cy is an optionally substituted ring selected from a 5-membered monocyclic heteroaromatic ring having 1-2 heteroatoms independently selected from nitrogen, oxygen, or sulfur. In some embodiments, -Cy is an optionally substituted ring selected from thiazolyl or imidazolyl. In some embodiments, -Cy is an optionally substituted ring selected from a 6-membered monocyclic heteroaromatic ring having 1-2 nitrogens. In some embodiments, -Cy is an optionally substituted ring selected from pyridyl.

In some embodiments, -Cy is an optionally substituted ring selected from a 4-8 membered saturated or partially unsaturated monocyclic heterocyclic ring having 1-2 heteroatoms independently selected from nitrogen, oxygen, or sulfur. In certain embodiments, -Cy is a 5-6 membered saturated monocyclic heterocyclic ring having 1 heteroatom independently selected from nitrogen, oxygen, or sulfur. In some embodiments, -Cy is pyranyl or tetrahydrofuranyl.

In some embodiments, R is an optionally substituted ring selected from

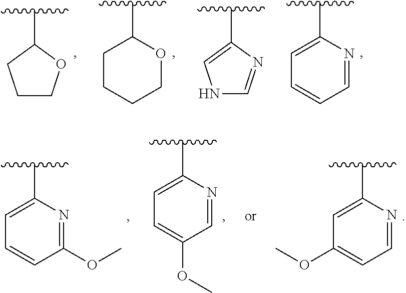

In certain embodiments, R¹ is selected from those depicted in Table 1, below.

In some embodiments, the present invention provides a compound of Formulae II-a or II-b:

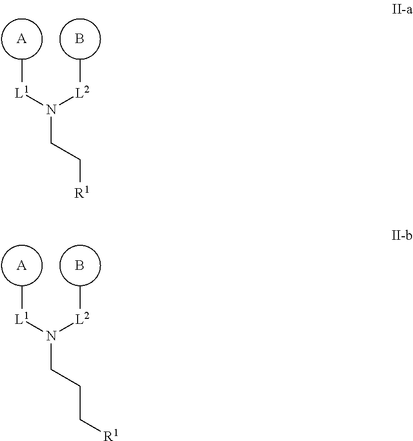

or a pharmaceutically acceptable salt thereof, wherein each variable is as defined above and described in embodiments herein, both singly and in combination.

In some embodiments, the present invention provides a compound of Formulae III-a or III-b:

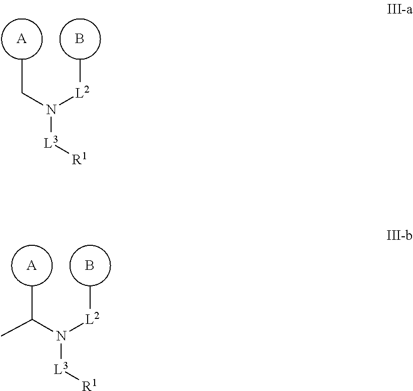

or a pharmaceutically acceptable salt thereof, wherein each variable is as defined above and described in embodiments herein, both singly and in combination.

In some embodiments, the present invention provides a compound of Formulae IV-a, IV-b, or IV-c:

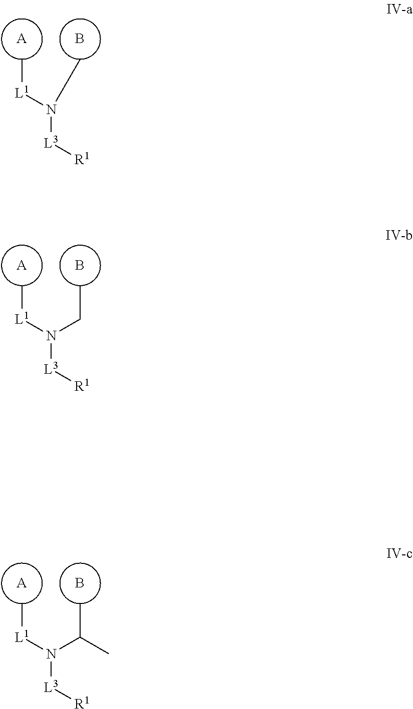

or a pharmaceutically acceptable salt thereof, wherein each variable is as defined above and described in embodiments herein, both singly and in combination.

In some embodiments, the present invention provides a compound of Formulae V-a or V-b:

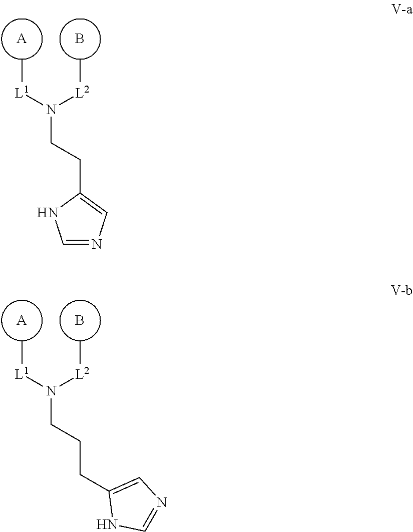

or a pharmaceutically acceptable salt thereof, wherein each variable is as defined above and described in embodiments herein, both singly and in combination.

In some embodiments, the present invention provides a compound of Formulae VI-a or VI-b:

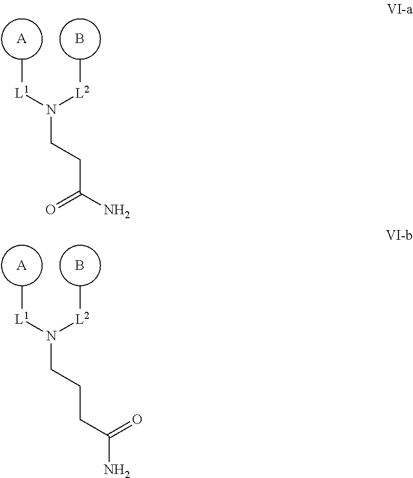

or a pharmaceutically acceptable salt thereof, wherein each variable is as defined above and described in embodiments herein, both singly and in combination.

In some embodiments, the present invention provides a compound of Formulae VII-a or VII-b:

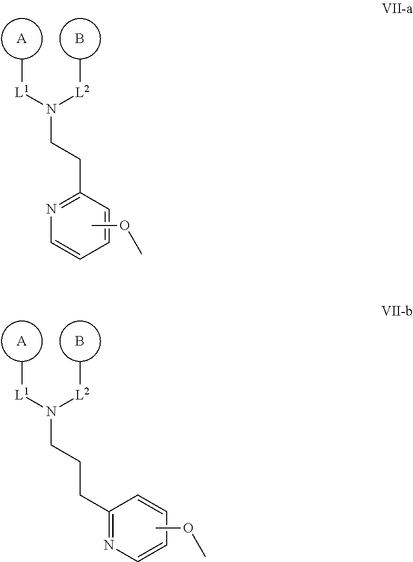

or a pharmaceutically acceptable salt thereof, wherein each variable is as defined above and described in embodiments herein, both singly and in combination.

In some embodiments, the present invention provides a compound of Formulae VIII-a, VIII-b, VIII-c or VIII-d:

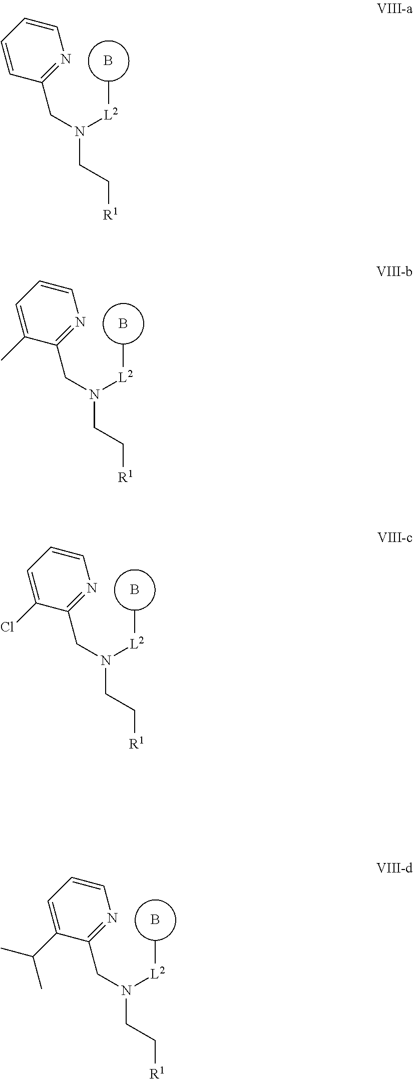

or a pharmaceutically acceptable salt thereof, wherein each variable is as defined above and described in embodiments herein, both singly and in combination.

In some embodiments, the present invention provides a compound of Formulae IX-a, IX-b, or IX-c:

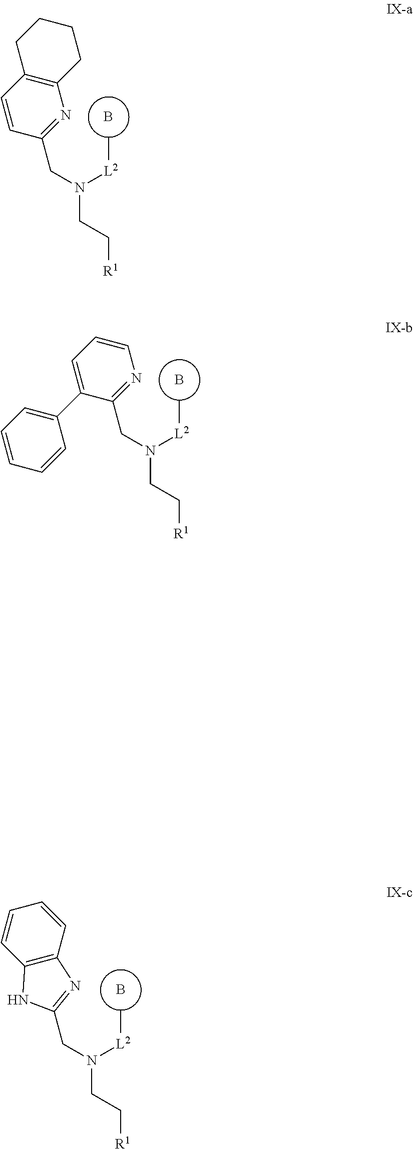

or a pharmaceutically acceptable salt thereof, wherein each variable is as defined above and described in embodiments herein, both singly and in combination.

In some embodiments, the present invention provides a compound of Formulae X-a, X-b, X-c or X-d:

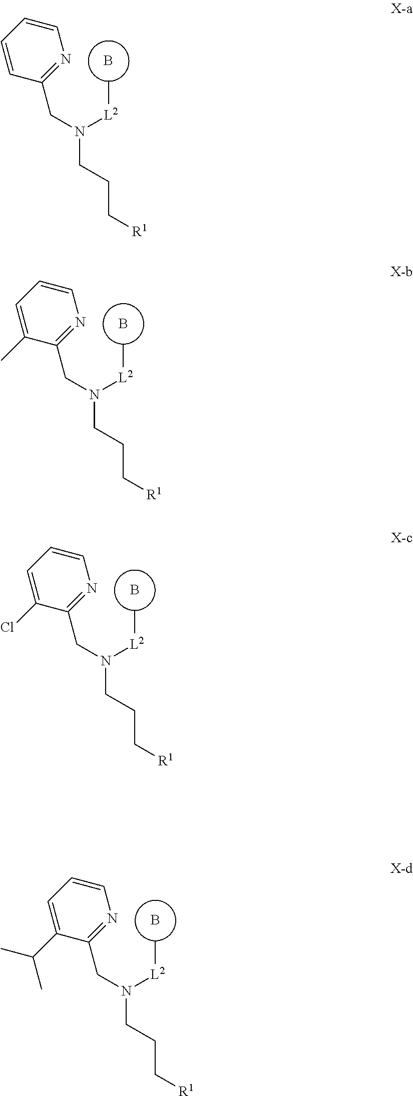

or a pharmaceutically acceptable salt thereof, wherein each variable is as defined above and described in embodiments herein, both singly and in combination.

In some embodiments, the present invention provides a compound of Formulae XI-a, XI-b, or XI-c:

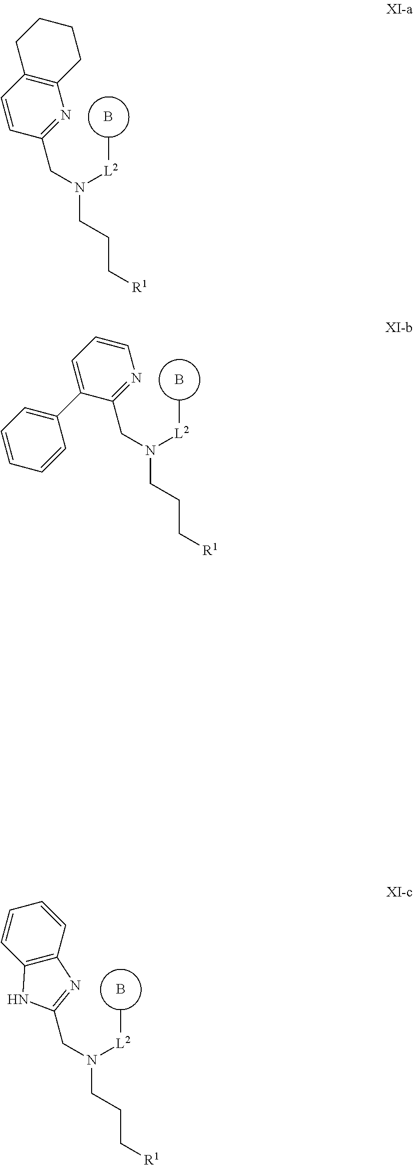

or a pharmaceutically acceptable salt thereof, wherein each variable is as defined above and described in embodiments herein, both singly and in combination.

In some embodiments, the present invention provides a compound of Formulae XII-a, XII-b, or XII-c:

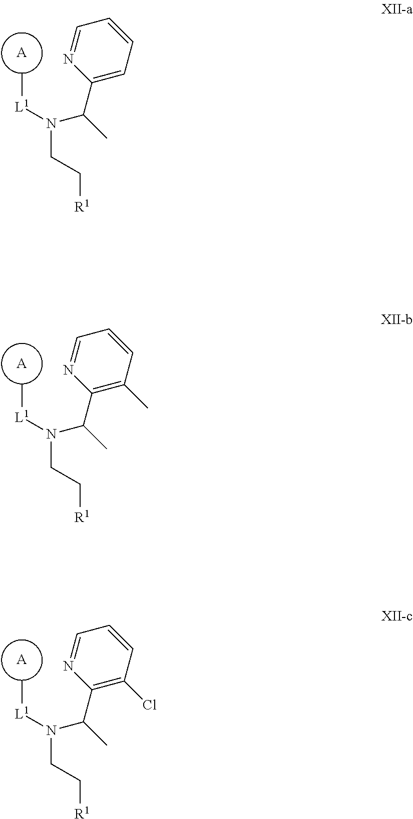

or a pharmaceutically acceptable salt thereof, wherein each variable is as defined above and described in embodiments herein, both singly and in combination.

In some embodiments, the present invention provides a compound of Formulae XIII-a, XIII-b, or XIII-c:

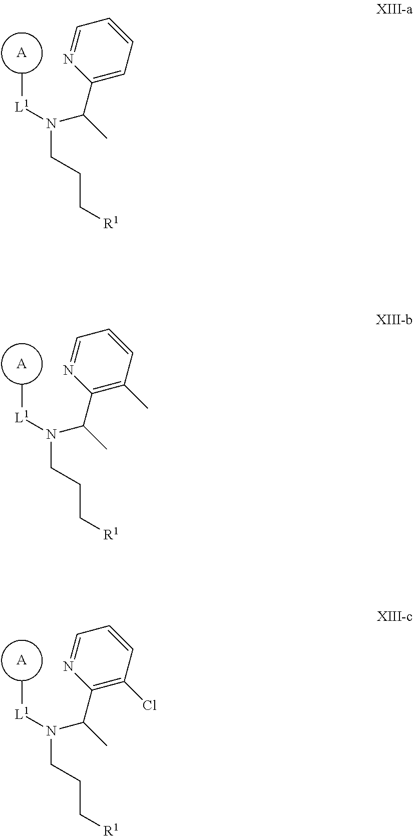

or a pharmaceutically acceptable salt thereof, wherein each variable is as defined above and described in embodiments herein, both singly and in combination.

In some embodiments, the present invention provides a compound of Formulae XIV-a, XIV-b, XIV-c, or XIV-d:

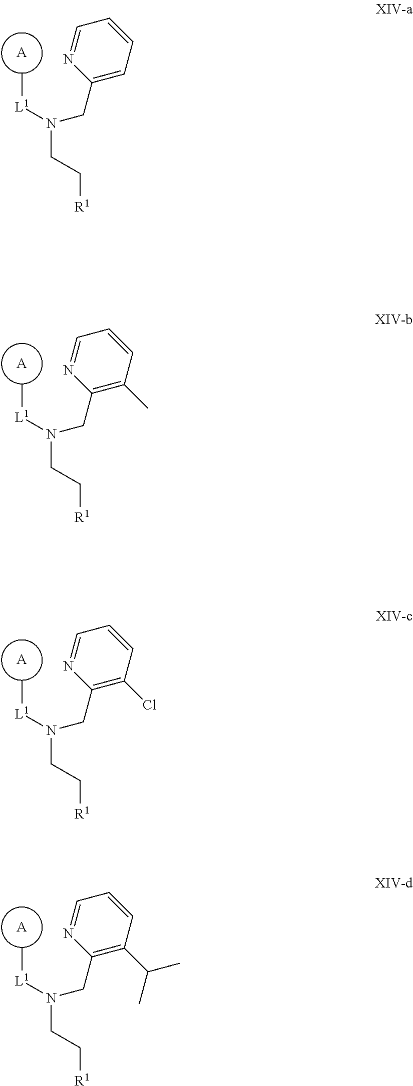

or a pharmaceutically acceptable salt thereof, wherein each variable is as defined above and described in embodiments herein, both singly and in combination.

In some embodiments, the present invention provides a compound of Formulae XV-a, XV-b, XV-c, or XV-d:

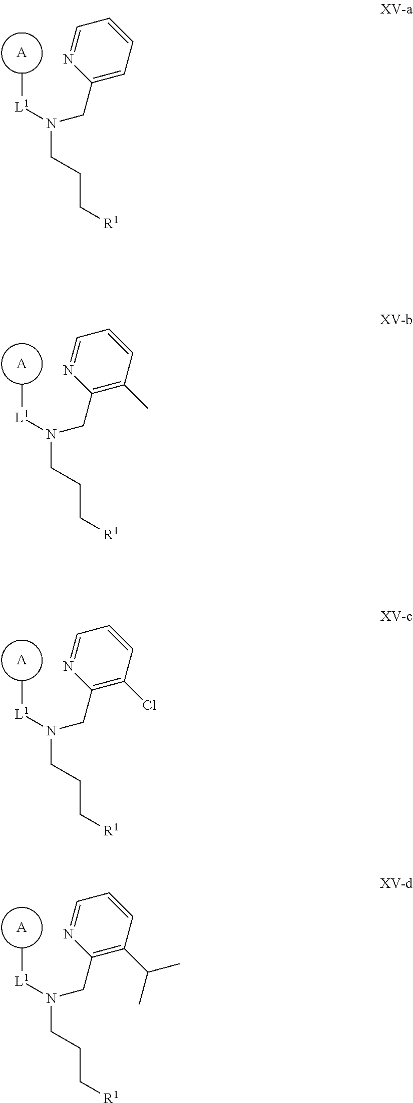

or a pharmaceutically acceptable salt thereof, wherein each variable is as defined above and described in embodiments herein, both singly and in combination.

In some embodiments, the present invention provides a compound of Formulae XVI-a or XVI-b:

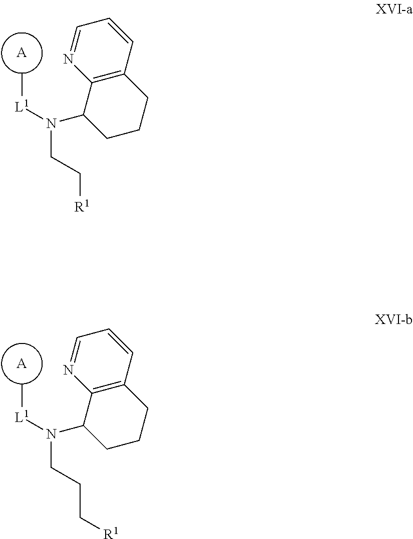

or a pharmaceutically acceptable salt thereof, wherein each variable is as defined above and described in embodiments herein, both singly and in combination.

In some embodiments, the present invention provides a compound of Formulae XVII-a or XVII-b:

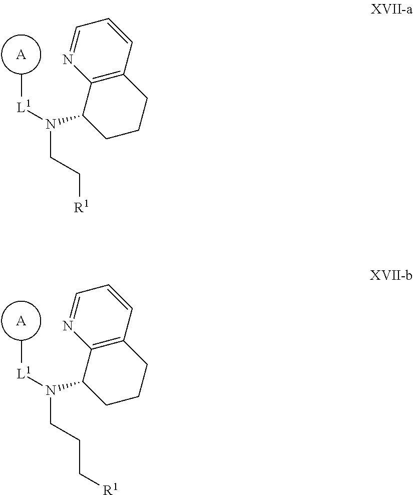

or a pharmaceutically acceptable salt thereof, wherein each variable is as defined above and described in embodiments herein, both singly and in combination.

Exemplary compounds of the invention are set forth in Table 1, below.

TABLE 1
Exemplary Compounds

| | |
|---|---|
| 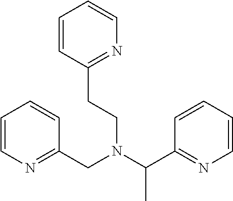 [SMILES: CC(c1ccccn1)N(CCc1ccccn1)Cc1ccccn1] | I-1 |
| 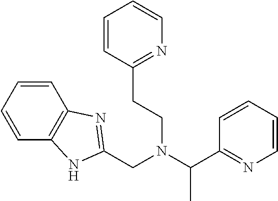 [SMILES: CC(c1ccccn1)N(CCc1ccccn1)Cc1nc2ccccc2[nH]1] | I-2 |
| 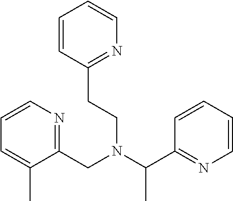 [SMILES: Cc1cccnc1CN(CCc1ccccn1)C(C)c1ccccn1] | I-3 |
| 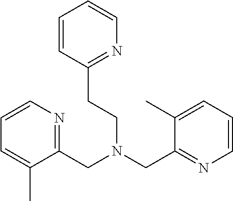 [SMILES: Cc1cccnc1CN(CCc1ccccn1)Cc1ncccc1C] | I-4 |
| 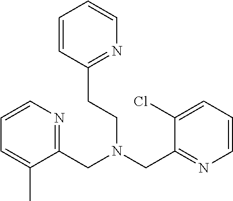 [SMILES: Cc1cccnc1CN(CCc1ccccn1)Cc1ncccc1Cl] | I-5 |
| 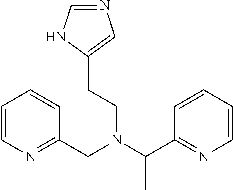 [SMILES: CC(c1ccccn1)N(CCc1cnc[nH]1)Cc1ccccn1] | I-6 |
| 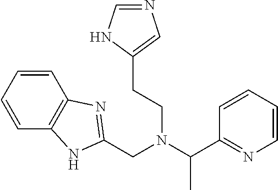 [SMILES: CC(c1ccccn1)N(CCc1cnc[nH]1)Cc1nc2ccccc2[nH]1] | I-7 |
| 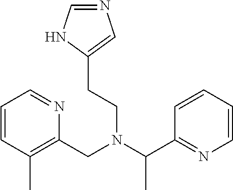 [SMILES: Cc1cccnc1CN(CCc1cnc[nH]1)C(C)c1ccccn1] | I-8 |
| 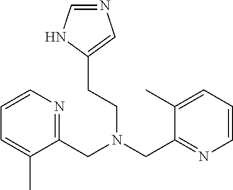 [SMILES: Cc1cccnc1CN(CCc1cnc[nH]1)Cc1ncccc1C] | I-9 |
| 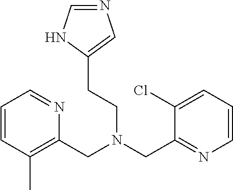 [SMILES: Cc1cccnc1CN(CCc1cnc[nH]1)Cc1ncccc1Cl] | I-10 |
| 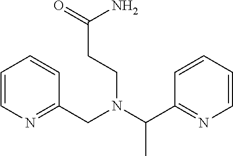 [SMILES: CC(c1ccccn1)N(CCC(N)=O)Cc1ccccn1] | I-11 |
| 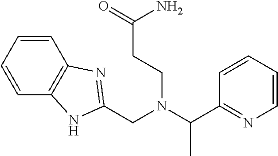 [SMILES: CC(c1ccccn1)N(CCC(N)=O)Cc1nc2ccccc2[nH]1] | I-12 |
| 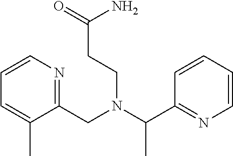 [SMILES: Cc1cccnc1CN(CCC(N)=O)C(C)c1ccccn1] | I-13 |
| 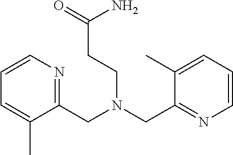 [SMILES: Cc1cccnc1CN(CCC(N)=O)Cc1ncccc1C] | I-14 |
| 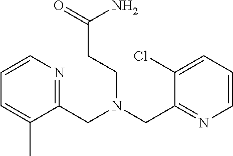 [SMILES: Cc1cccnc1CN(CCC(N)=O)Cc1ncccc1Cl] | I-15 |
| 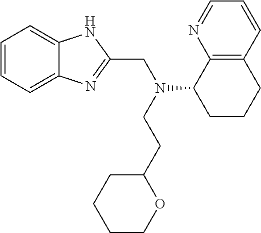 [SMILES: c1cnc2c(c1)CCC[C@@H]2N(CCC1CCCCO1)Cc1nc2ccccc2[nH]1] | I-16 |
| 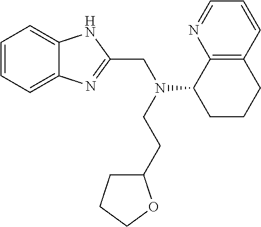 [SMILES: c1cnc2c(c1)CCC[C@@H]2N(CCC1CCCO1)Cc1nc2ccccc2[nH]1] | I-17 |
| 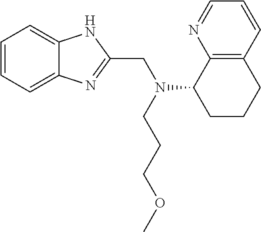 [SMILES: COCCCN(Cc1nc2ccccc2[nH]1)[C@H]1CCCc2cccnc21] | I-18 |
| 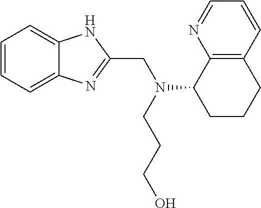 [SMILES: OCCCN(Cc1nc2ccccc2[nH]1)[C@H]1CCCc2cccnc21] | I-19 |
| 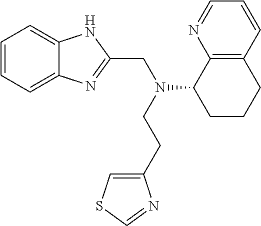 [SMILES: c1cnc2c(c1)CCC[C@@H]2N(CCc1cscn1)Cc1nc2ccccc2[nH]1] | I-20 |
| 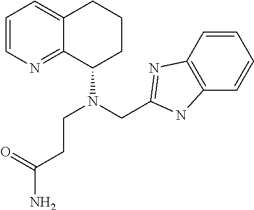 [SMILES: NC(=O)CCN(Cc1nc2ccccc2[nH]1)[C@H]1CCCc2cccnc21] | I-21 |
| 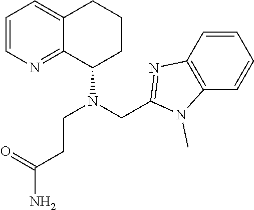 [SMILES: Cn1c(CN(CCC(N)=O)[C@H]2CCCc3cccnc32)nc2ccccc21] | I-22 |
| 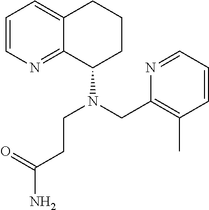 [SMILES: Cc1cccnc1CN(CCC(N)=O)[C@H]1CCCc2cccnc21] | I-23 |
| 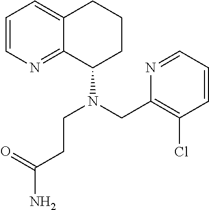 [SMILES: NC(=O)CCN(Cc1ncccc1Cl)[C@H]1CCCc2cccnc21] | I-24 |
| 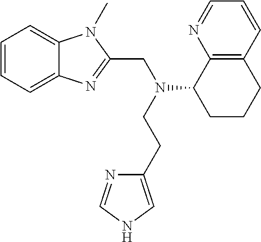 [SMILES: Cn1c(CN(CCc2c[nH]cn2)[C@H]2CCCc3cccnc32)nc2ccccc21] | I-25 |
| 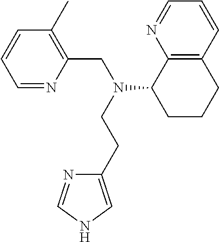 [SMILES: Cc1cccnc1CN(CCc1c[nH]cn1)[C@H]1CCCc2cccnc21] | I-26 |
| 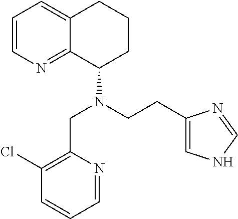 [SMILES: Clc1cccnc1CN(CCc1c[nH]cn1)[C@H]1CCCc2cccnc21] | I-27 |
| 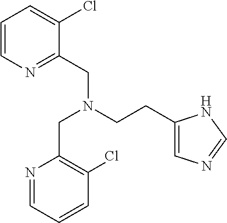 [SMILES: Clc1cccnc1CN(CCc1cnc[nH]1)Cc1ncccc1Cl] | I-28 |
| 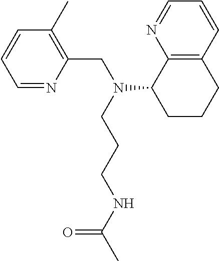 [SMILES: CC(=O)NCCCN(Cc1ncccc1C)[C@H]1CCCc2cccnc21] | I-29 |
| 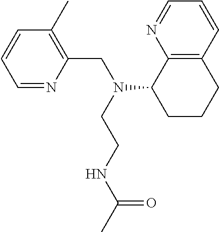 [SMILES: CC(=O)NCCN(Cc1ncccc1C)[C@H]1CCCc2cccnc21] | I-30 |
| 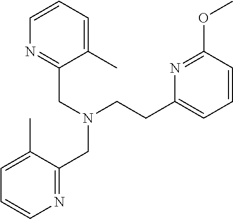 [SMILES: COc1cccc(CCN(Cc2ncccc2C)Cc2ncccc2C)n1] | I-31 |
| 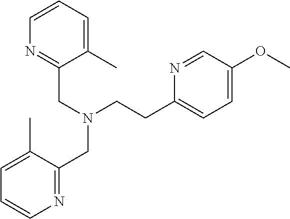 [SMILES: COc1ccc(CCN(Cc2ncccc2C)Cc2ncccc2C)nc1] | I-32 |
| 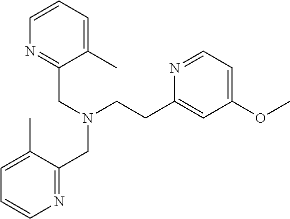 [SMILES: COc1ccnc(CCN(Cc2ncccc2C)Cc2ncccc2C)c1] | I-33 |
| 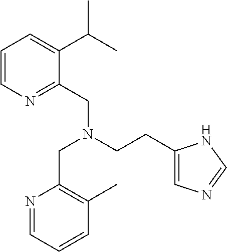 [SMILES: Cc1cccnc1CN(CCc1cnc[nH]1)Cc1ncccc1C(C)C] | I-34 |
| 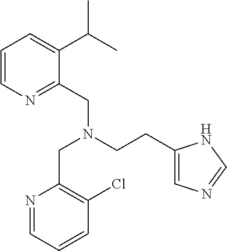 [SMILES: CC(C)c1cccnc1CN(CCc1cnc[nH]1)Cc1ncccc1Cl] | I-35 |
| 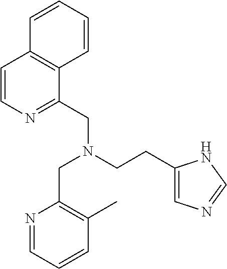 [SMILES: Cc1cccnc1CN(CCc1cnc[nH]1)Cc1nccc2ccccc12] | I-36 |
| 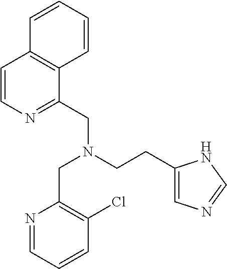 [SMILES: Clc1cccnc1CN(CCc1cnc[nH]1)Cc1nccc2ccccc12] | I-37 |
| 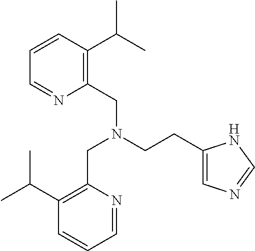 [SMILES: CC(C)c1cccnc1CN(CCc1cnc[nH]1)Cc1ncccc1C(C)C] | I-38 |
| 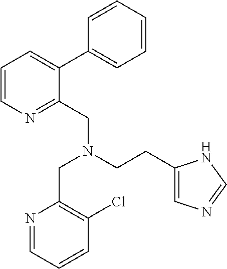 [SMILES: Clc1cccnc1CN(CCc1cnc[nH]1)Cc1ncccc1-c1ccccc1] | I-39 |
| 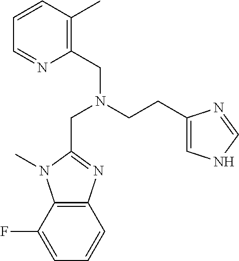 [SMILES: Cc1cccnc1CN(CCc1c[nH]cn1)Cc1nc2cccc(F)c2n1C] | I-40 |
| 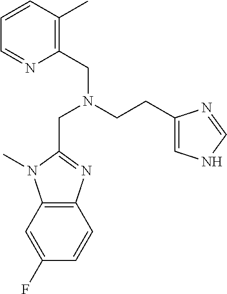 [SMILES: Cc1cccnc1CN(CCc1c[nH]cn1)Cc1nc2ccc(F)cc2n1C] | I-41 |
| 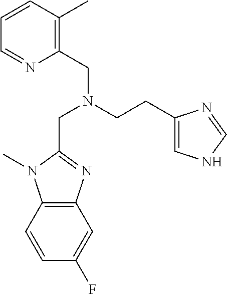 [SMILES: Cc1cccnc1CN(CCc1c[nH]cn1)Cc1nc2cc(F)ccc2n1C] | I-42 |
| 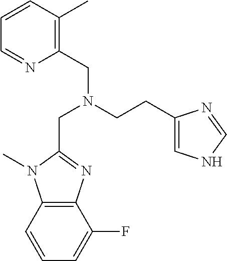 [SMILES: Cc1cccnc1CN(CCc1c[nH]cn1)Cc1nc2c(F)cccc2n1C] | I-43 |
| 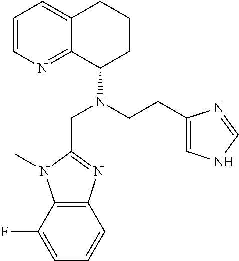 [SMILES: Cn1c(CN(CCc2c[nH]cn2)[C@H]2CCCc3cccnc32)nc2cccc(F)c21] | I-44 |
| 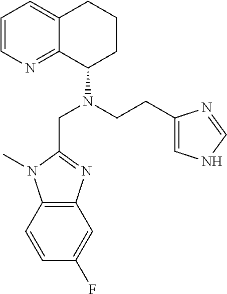 [SMILES: Cn1c(CN(CCc2c[nH]cn2)[C@H]2CCCc3cccnc32)nc2cc(F)ccc21] | I-45 |
| 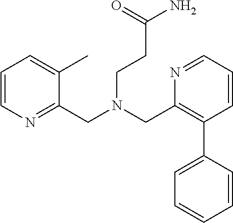 [SMILES: Cc1cccnc1CN(CCC(N)=O)Cc1ncccc1-c1ccccc1] | I-46 |
| 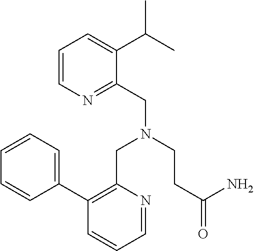 [SMILES: CC(C)c1cccnc1CN(CCC(N)=O)Cc1ncccc1-c1ccccc1] | I-47 |
| 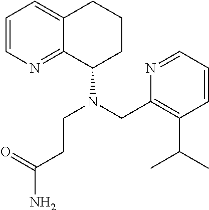 [SMILES: CC(C)c1cccnc1CN(CCC(N)=O)[C@H]1CCCc2cccnc21] | I-48 |
| 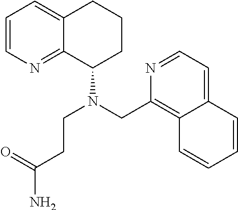 [SMILES: NC(=O)CCN(Cc1nccc2ccccc12)[C@H]1CCCc2cccnc21] | I-49 |
| 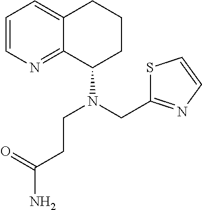 [SMILES: NC(=O)CCN(Cc1nccs1)[C@H]1CCCc2cccnc21] | I-50 |
| 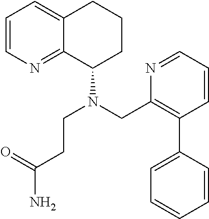 [SMILES: NC(=O)CCN(Cc1ncccc1-c1ccccc1)[C@H]1CCCc2cccnc21] | I-51 |
| 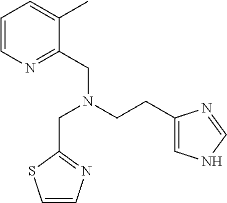 [SMILES: Cc1cccnc1CN(CCc1c[nH]cn1)Cc1nccs1] | I-52 |
| 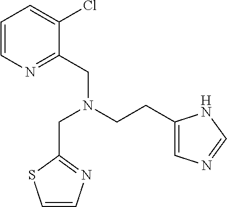 [SMILES: Clc1cccnc1CN(CCc1cnc[nH]1)Cc1nccs1] | I-53 |
| 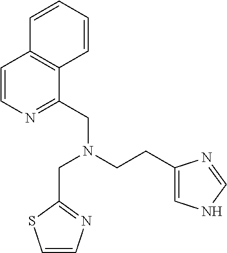 [SMILES: c1ccc2c(CN(CCc3c[nH]cn3)Cc3nccs3)nccc2c1] | I-54 |
| 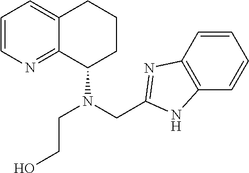 [SMILES: OCCN(Cc1nc2ccccc2[nH]1)[C@H]1CCCc2cccnc21] | I-55 |
| 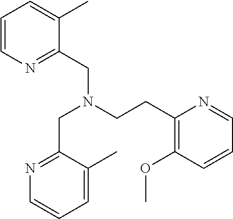 [SMILES: COc1cccnc1CCN(Cc1ncccc1C)Cc1ncccc1C] | I-56 |
| 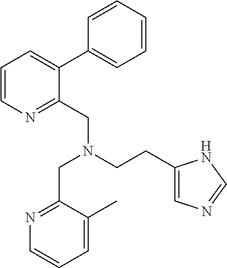 [SMILES: Cc1cccnc1CN(CCc1cnc[nH]1)Cc1ncccc1-c1ccccc1] | I-57 |
| 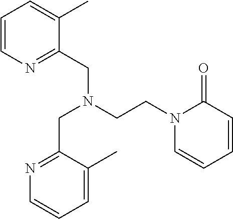 [SMILES: Cc1cccnc1CN(CCn1ccccc1=O)Cc1ncccc1C] | I-58 |
| 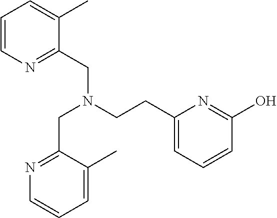 [SMILES: Cc1cccnc1CN(CCc1cccc(O)n1)Cc1ncccc1C] | I-59 |
| 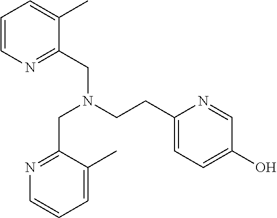 [SMILES: Cc1cccnc1CN(CCc1ccc(O)cn1)Cc1ncccc1C] | I-60 |
| 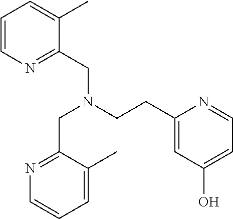 [SMILES: Cc1cccnc1CN(CCc1cc(O)ccn1)Cc1ncccc1C] | I-61 |
| 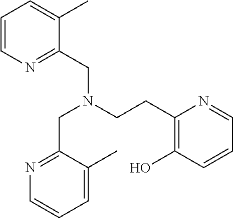 [SMILES: Cc1cccnc1CN(CCc1ncccc1O)Cc1ncccc1C] | I-62 |
| 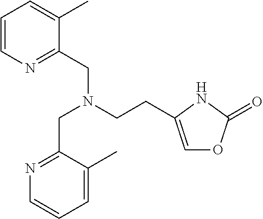 [SMILES: Cc1cccnc1CN(CCc1coc(=O)[nH]1)Cc1ncccc1C] | I-63 |
| 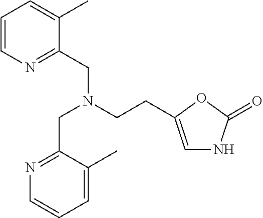 [SMILES: Cc1cccnc1CN(CCc1c[nH]c(=O)o1)Cc1ncccc1C] | I-64 |
| 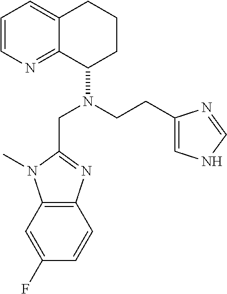 | I-65 |
| 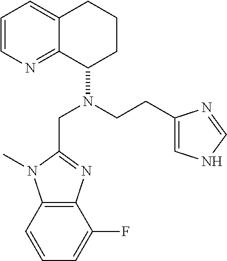 | I-66 |
| 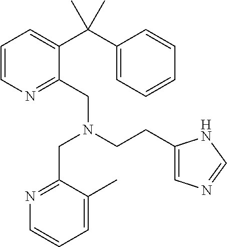 [SMILES: Cc1cccnc1CN(CCc1cnc[nH]1)Cc1ncccc1C(C)(C)c1ccccc1] | I-67 |
| 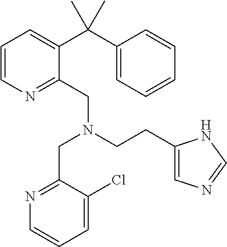 [SMILES: CC(C)(c1ccccc1)c1cccnc1CN(CCc1cnc[nH]1)Cc1ncccc1Cl] | I-68 |
| 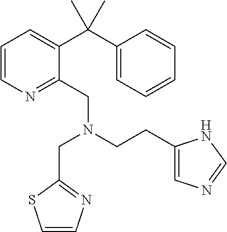 [SMILES: CC(C)(c1ccccc1)c1cccnc1CN(CCc1cnc[nH]1)Cc1nccs1] | I-69 |

In some embodiments, the present invention provides a compound set forth in Table 1, above, or a pharmaceutically acceptable salt thereof.

It has been surprisingly found that provided compounds are particularly useful for treating cancers such as a brain cancer, for example glioblastoma. In particular, it has been surprisingly found that provided compounds have improved ability to cross the blood brain barrier and reside therein for an enhanced amount of time as compared with other CXCR4 inhibitors. In some embodiments, a provided compound provides a residence time sufficient to effect a therapeutic result without excessive accumulation of drug in the brain. Methods of treating glioblastoma and other cancers may be performed similarly to related methods known in the art. Methods of evaluating efficacy of the disclosed compounds may be performed similarly to related methods known in the art. See, e.g., van den Bent, M. et al., Cancer Chemother Pharmacol (2017) 80:1209-1217 and Mrugala, M. M., Discovery Medicine (2013) 83:221-220 which are hereby incorporated by reference; as well as other methods known in the art.

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit CXCR4, or a mutant thereof, in a biological sample or in a patient. In certain embodiments, the amount of compound in compositions of this invention is such that is effective to measurably inhibit CXCR4, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of CXCR4, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the inhibition of CXCR4 or a mutant thereof.

The activity of a compound utilized in this invention as an inhibitor of CXCR4, or a mutant thereof, may be assayed in vitro, in vivo or in a cell line. In vitro assays include assays that determine inhibition of CXCR4, or a mutant thereof. Alternate in vitro assays quantitate the ability of the inhibitor to bind to CXCR4. Detailed conditions for assaying a compound utilized in this invention as an inhibitor of CXCR4, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of CXCR4 and are therefore useful for treating one or more disorders associated with activity of CXCR4. Thus, in certain embodiments, the present invention provides a method for treating a CXCR4-mediated disorder comprising the step of administering to a patient in need thereof a compound of the present invention, or pharmaceutically acceptable composition thereof.

As used herein, the terms “CXCR4-mediated” disorders, diseases, and/or conditions as used herein means any disease or other deleterious condition in which CXCR4, or a mutant thereof, is known to play a role. Accordingly, another embodiment of the present invention relates to treating or lessening the severity of one or more diseases in which CXCR4, or a mutant thereof, are known to play a role.

In some embodiments, the present invention provides a method for treating one or more disorders, diseases, and/or conditions wherein the disorder, disease, or condition includes, but is not limited to, a cellular proliferative disorder.

### Cellular Proliferative Disorders

The present invention features methods and compositions for the diagnosis and prognosis of cellular proliferative disorders (e.g., cancer) and the treatment of these disorders by targeting CXCR4. Cellular proliferative disorders described herein include, e.g., cancer, obesity, and proliferation-dependent diseases. Such disorders may be diagnosed using methods known in the art.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g., Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, the present invention provides a method for treating a tumor in a patient in need thereof, comprising administering to the patient any of the compounds, salts or pharmaceutical compositions described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the the tumor comprises small cell lung cancer (SCLC). In some embodiments the the tumor comprises non-small cell lung cancer (NSCLC).

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

### Primary Immune Deficiencies

In some embodiments, the present invention provides a method for treating one or more disorders, diseases, and/or conditions wherein the disorder, disease, or condition includes, but is not limited to, a primary immunodeficiency disease or disorder, comprising administering to a patient in need thereof an effective amount of a disclosed compound. Primary immune deficiencies treatable by the methods of the present invention include: warts, hypogammaglobulinemia, infections, myelokathexis (WHIMs) syndrome; severe congenital neutropenia (SCN), especially those arising from G6PC3 deficiency (McDermott et al. (2010) Blood 116:2793-2802); GATA2 deficiency (Mono MAC syndrome) (Maciejweski-Duval et al. (2015) J. Leukoc. Biol. 5MA0815-288R (Epub. ahead of printing); idiopathic CD4+T lymphocytopenia (ICL); and Wiskott-Aldrich Syndrome.

The compounds and compositions, according to the method of the present invention, may be administered using any amount and any route of administration effective for treating or lessening the severity of a cancer, an autoimmune disorder, a primary immune deficiency, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting CXCR4 activity in a biological sample comprising the step of contacting said biological sample with a compound of this invention, or a composition comprising said compound.

According to another embodiment, the invention relates to a method of inhibiting CXCR4, or a mutant thereof, activity in a biological sample comprising the step of contacting said biological sample with a compound of this invention, or a composition comprising said compound. In certain embodiments, the invention relates to a method of irreversibly inhibiting CXCR4, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof, and blood, saliva, urine, feces, semen, tears, or other body fluids or extracts thereof.

Another embodiment of the present invention relates to a method of inhibiting CXCR4 in a patient comprising the step of administering to said patient a compound of the present invention, or a composition comprising said compound.

According to another embodiment, the invention relates to a method of inhibiting CXCR4, or a mutant thereof, activity in a patient comprising the step of administering to said patient a compound of the present invention, or a composition comprising said compound. According to certain embodiments, the invention relates to a method of irreversibly inhibiting CXCR4, or a mutant thereof, activity in a patient comprising the step of administering to said patient a compound of the present invention, or a composition comprising said compound. In other embodiments, the present invention provides a method for treating a disorder mediated by CXCR4, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to checkpoint inhibitors; aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal©); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signalling processes should proceed.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (ap) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L¹ monoclonal Antibody (Anti-B7-H1; MEDI4736), MK-3475 (PD-1 blocker), Nivolumab (anti-PD antibody), CT-011 (anti-PDl antibody), BY55 monoclonal antibody, AMP224 (anti-PDLI antibody), BMS-936559 (anti-PDLI antibody), MPLDL3280A (anti-PDLI antibody), MSB0010718C (anti-PDLI antibody), and ipilimumab (anti-CTLA-4 checkpoint inhibitor). Checkpoint protein ligands include, but are not limited to PD-L¹, PD-L², B7-H3, B7-H4, CD28, CD86 and TIM-3.

In certain embodiments, the immune checkpoint inhibitor is selected from a PD-1 antagonist, a PD-L¹ antagonist, and a CTLA-4 antagonist. In some embodiments, the checkpoint inhibitor is selected from the group consisting of nivolumab (Opdivo®), atezolizumab (Tecentriq®), avelumab (Bavencio®), durvalumab (Imfinzi®), ipilimumab (Yervoy®), and pembrolizumab (Keytruda®).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g., under the trademark Carboplat™. Oxaliplatin can be administered, e.g., in the form as it is marketed, e.g., under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-R, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; sis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFRi ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C₁₋₁₀₃₃, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (P13K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g., inhibitors of phosphatase 1, phosphatase 2A, or CDC25, such as okadaic acid or a derivative thereof.

Compounds which induce cell differentiation processes include, but are not limited to, retinoic acid, α- γ- or δ- tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R¹¹⁵⁷⁷⁷ (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan*), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### EXEMPLIFICATION

### General Synthetic Methods

The following examples are intended to illustrate the invention and are not to be construed as being limitations thereon. Unless otherwise stated, one or more tautomeric forms of compounds of the examples described hereinafter may be prepared in situ and/or isolated. All tautomeric forms of compounds of the examples described hereafter should be considered to be disclosed. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Abbreviations

equiv or eq: molar equivalents
rt: room temperature
UV: ultra violet
HPLC: high pressure liquid chromatography
Rt: retention time
LCMS or LC-MS: liquid chromatography-mass spectrometry
NMR: nuclear magnetic resonance
CC: column chromatography
TFA: trifluoroacetic acid
TLC: thin layer chromatography
sat: saturated
aq: aqueous
Ac: acetyl
DCM: dichloromethane
DCE: dichloroethane
DEA: diethylamine
DMF: dimethylformamide
DMSO: dimethylsulfoxide
ACN or MeCN: acetonitrile
DIPEA: diisopropylethylamine
EA or EtOAc: ethyl acetate
BINAP: (±)-2,2′-Bis(diphenylphosphino)-1,1′-binaphthalene
TEA: triethylamine
THF: tetrahydrofuran
TBS: tert-butyldimethylsilyl
KHMDS: potassium hexamethyl disilylazide
Tf: trifluoromethanesulfonate
Ms: methanesulfonyl
NBS: N-bromosuccinimide
PE: petroleum ether
TFA: trifluoroacetic acid
MMPP: magnesium monoperoxyphthalate
HATU: 1-[Bis(dimethylamino)methylene]-1H-1,2,3-triazolo[4,5-b]pyridinium 3-oxid Hexafluorophosphate
Tol: toluene
Trt: Trityl
SEM: [2-(Trimethylsilyl)ethoxy]methyl

General Information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (200-300 mesh). Solvent systems are reported as mixtures by volume. All ¹H NMR spectra were recorded on a Bruker 400 (400 MHz) spectrometer. ¹H chemical shifts are reported in 6 values in parts per million (ppm) with the deuterated solvent as the internal standard. Data are reported as follows: chemical shift, multiplicity (s=singlet, d=doublet, t=triplet, q=quartet, br=broad, m=multiplet), coupling constant (Hz), integration (i.e. number of protons). LCMS spectra were obtained on an Agilent 1200 series 6110 or 6120 mass spectrometer with electrospray ionization and except as otherwise indicated, the general LCMS conditions were as follows: Waters X Bridge C18 column (50 mm*4.6 mm*3.5 μm), Flow Rate: 2.0 mL/min, the column temperature: 40° C.

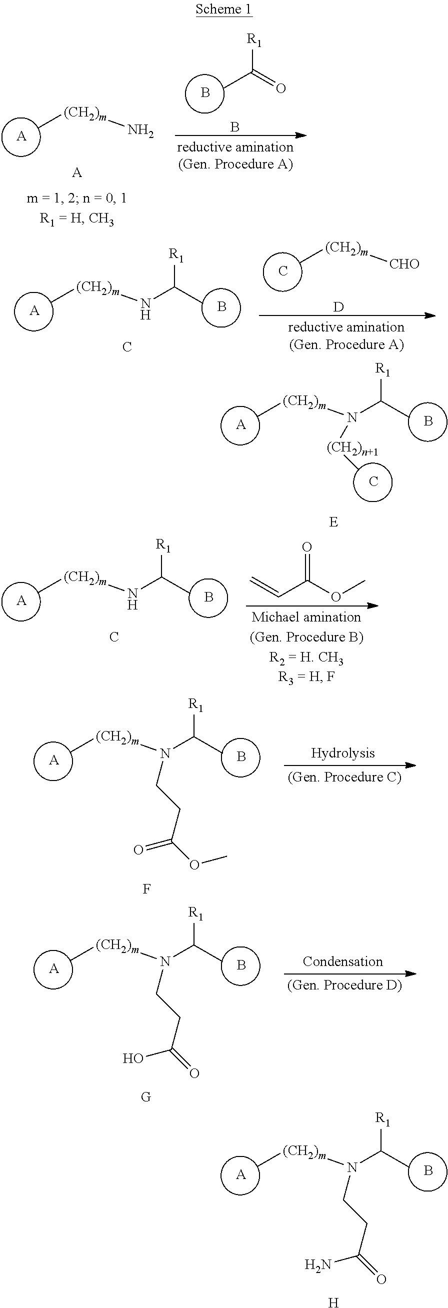

In Scheme 1 above, Cycle A, B are nitrogen-containing heterocycle compounds, such as non-substituted or substituted pyridine, imidazole, thiazole, quinoline and isoquinoline. Cycle C are heterocycle compounds, such as non-substituted or substituted pyridine, imidazole, thioquinoline, isoquinoline, tetrahydrofuran and tetrahydropyran.

As shown generally in Scheme 1, an amine according to structure A may be condensed with an aldehyde or ketone according to structure B to yield intermediate C, for example by following General Procedures A. Condensation with an aldehyde according to structure C also following General Procedures A provides compounds of structure E. Addition to methyl acrylate following General Procedure B, structure C is converted to structure F. Carboxylic acid G was obtained following General Procedure C. Finally, amide H was formed following General Procedure D. The General Procedures are described in more detail in the Exemplification, below.

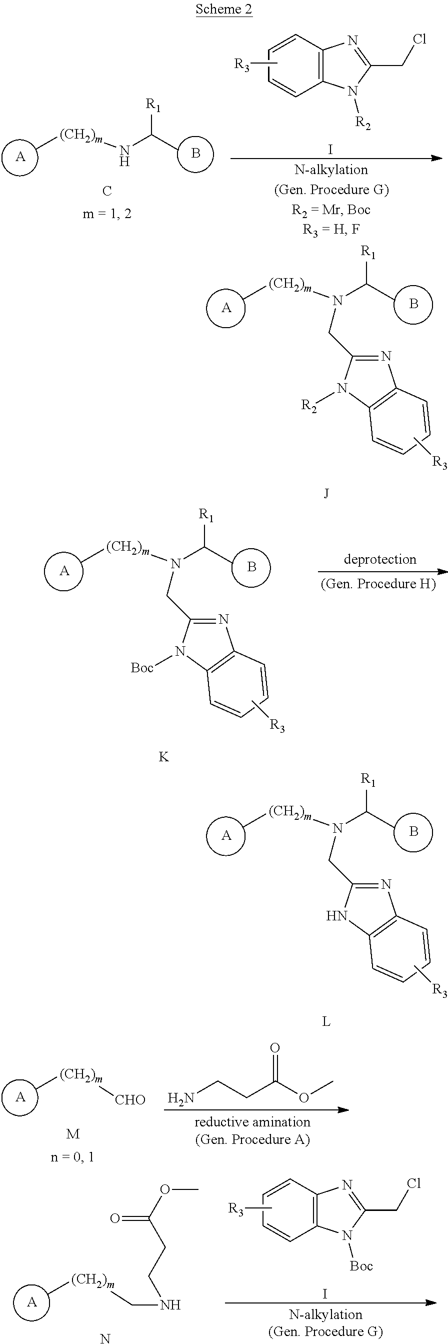

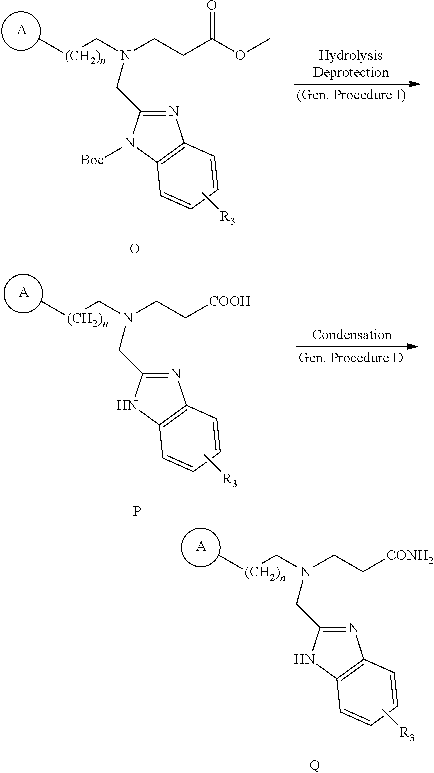

In Scheme 2 above, Cycle A, B are nitrogen-containing hetercycle compounds, such as non-substituted or substituted pyridine, imidazole, thiazole, quinoline and isoquinoline.

As shown generally in Scheme 2, alkylation with 2-(chloromethyl)-1H-benzo[d]imidazole derivatives following General Procedure G, intermediate C is converted to structure J or K Structure K is converted to structure L after deprotection. With the similar method, structure Q is formed from an aldehyde according to the structure M through intermediates N, O and P. The General Procedures are described in more detail in the Exemplification, below.

General Procedure A (reductive amination of primary amine to secondary amine): To a mixture of primary amine (concentration 0.1˜1 M), corresponding aldehyde or ketone (1˜2 eq) and sodium cyanoborohydride (2 eq) in MeOH was added several drops of acetic acid, and then the mixture was stirred at room temperature for 2˜18 h. The mixture was neutralized by saturated NaHCO₃ aqueous solution to pH=8-9 and extracted by DCM. The organic layers were washed with brine, dried over Na₂SO₄, filtered and concentrated in vacuum to give secondary amine.

General Procedure B (Michael addition of methyl acrylate by secondary amine): A mixture of the secondary (concentration 0.1˜1 M), methyl acrylate (4 eq) in MeOH was stirred at 80° C. overnight in a sealed tube. After the reaction was completed, the solvent was evaporated in vacuum to give a residue, which was purified by CC (DCM:MeOH=50:1) to give the desired tertiary amine.

General Procedure C (hydrolysis of the methyl carboxylic ester to carboxylic acid): To a solution of the methyl carboxylic ester (concentration 0.1˜1 M) in MeOH was added 1N NaOH aq (3 eq), and the mixture was stirred at room temperature for 30 min. Then the mixture was acidified with con. HCl and concentrated in vacuum to give the desired carboxylic acid.

General Procedure D (condensation of carboxylic acid and ammonium chloride): A mixture of the carboxylic acid (concentration 0.1˜1 M), TEA (6 eq), HATU (1.5 eq) in DMF was stirred at room temperature for 20 min, followed by adding ammonium chloride (3 mmol), and the reaction mixture was stirred at room temperature for 2 h. After the reaction was completed, the mixture was quenched with H₂O and extracted with DCM. The combined organic phase was dried over anhydrous Na₂SO₄, filtered and concentrated to give a residue, which was purified by prep-HPLC to give desired amide.

General Procedure E (Trt cleavage of N-trt protected imidazole catalogs): To a solution of N-trt protected amine (concentration 0.1˜1 M) in DCM was added TFA (1/15 volume of DCM) at room temperature. The reaction mixture was stirred for 2 h, then concentrated and saturated NaHCO₃ aqueous solution was added and the mixture was extracted with DCM. The organic extracts were dried over Na₂SO₄, filtered and concentrated to give crude free amine, which was purified by prep-HPLC to give desired target.

General Procedure F ((S)-methyl PMB deprotection): To a solution of N—(S)-methyl PMB protected amine (concentration 0.1˜1 M) in DCM was added TFA (1/15 volume of DCM) at room temperature. The reaction mixture was stirred for 2 h, then concentrated and saturated NaHCO₃ aqueous solution was added and the mixture was extracted with DCM. The organic extracts were dried over Na₂SO₄, filtered and concentrated to give crude free amine, which was purified by CC to give desired target.

General Procedure G (N-alkylation of the secondary amine with chloromethyl-N-contained heterocycle derivatives): A mixture of the secondary amine (concentration 0.1-1.0 M), chloromethyl-N-contained heterocycle derivatives (1.1 eq), DIPEA (2 eq), KI (0.1 eq) in MeOH was stirred at 60° C. overnight. After the reaction was completed, the mixture was concentrated in vacuum, diluted with water and extracted with DCM. The combined organic phase was dried over Na₂SO₄, filtered and concentrated to give a residue, which was purified by CC to give desired tertiary amine.

General Procedure H (Boc cleavage of N-Boc protected amines): To a solution of N-Boc protected amine (concentration 0.1˜1 M) in DCM was added TFA (1/15 volume of DCM) at room temperature. The reaction mixture was stirred for 2 h, then concentrated and saturated NaHCO₃ aqueous solution was added and the mixture was extracted with DCM. The organic extracts were dried over Na₂SO₄, filtered and concentrated to give crude free amine, which was purified by prep-HPLC to give desired target.

General Procedure I (hydrolysis of the methyl carboxylic ester to carboxylic acid and Boc cleavage of N-Boc protected amines in one step): To a solution of N-Boc protected amino carboxylic ester (concentration 0.1-1.0 M) in MeOH was added NaOH aq (1 M, 2 eq), and the mixture was stirred at room temperature for 30 min. Then the mixture was acidified with con. HCl (0.2 mL) and concentrated in vacuum to give desired amino carboxylic acid as a white solid, which was used in the next step directly.

### Example 1: Synthesis of I-1

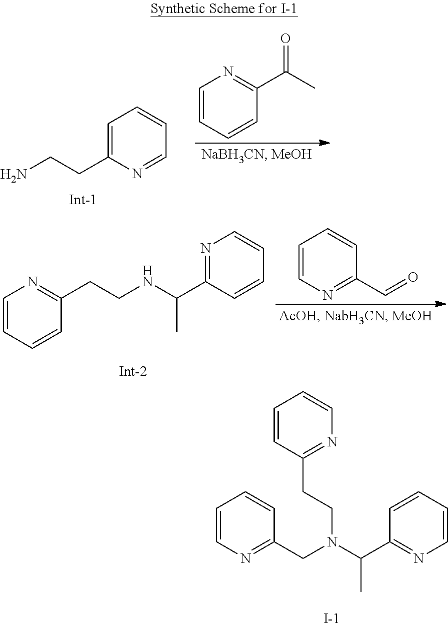

(2-(Pyridin-2-yl)-N-(1-(pyridin-2-yl)ethyl)ethan-1-amine): Following general procedure A, Int-2 (1.1 g, 59% yield) was obtained as yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 91%; Rt=1.44 min; MS Calcd.: 227.1; MS Found: 228.1[M+H]⁺.

2-(Pyridin-2-yl)-N-(1-(pyridin-2-yl)ethyl)-N-(pyridin-2-ylmethyl)ethan-1-amine: Following general procedure A, I-1 (21 mg, 7.5% yield) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 97.2%; Rt=1.57 min; MS Calcd.: 318.1; MS Found: 319.2 [M+H]⁺. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: 100%; Rt=7.65 min. ¹H NMR (400 MHz, CD₃OD) δ: 8.49-8.46 (m, 1H), 8.41-8.39 (m, 1H), 8.37-8.35 (m, 1H), 7.76-7.69 (m, 3H), 7.41 (dd, J=12.4, 8.0 Hz, 2H), 7.29-7.19 (m, 4H), 4.15 (q, J=13.6 Hz, 1H), 3.95 (d, J=15.2 Hz, 1H), 3.80 (d, J=15.2 Hz, 1H), 3.08-3.00 (m, 1H), 2.95 (t, J=6.8 Hz, 2H), 2.90-2.83 (m, 1H), 1.47 (d, J=6.8 Hz, 3H).

### Example 2: Synthesis of I-2

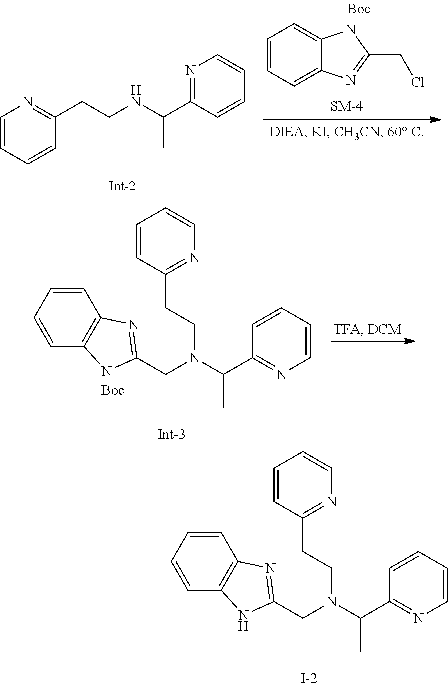

Tert-butyl 2-(((1-(pyridin-2-yl)ethyl)(2-(pyridin-2-yl)ethyl)amino)methyl)-1H-benzo[d]imidazole-1-carboxylate: Following general procedure G, Int-3 (120 mg, yield: 60%) was obtained as yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 82%; Rt=1.92 min; MS Calcd.: 357.2; MS Found: 358.3[M+H]*.

N-((1H-benzo[d]imidazol-2-yl)methyl)-2-(pyridin-2-yl)-N-(1-(pyridin-2-yl)ethyl)ethanamine: Following general procedure H, I-2 (15 mg, 16% yield) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 97.4%; Rt=1.62 min; MS Calcd.: 357.5; MS Found: 358.2 [M+H]*. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: 100%; Rt=8.02 min. ¹H NMR (400 MHz, CD₃OD) δ: 8.48 (dd, J=4.8, 0.8 Hz, 1H), 8.38 (dd, J=4.8, 0.8 Hz, 1H), 7.73-7.68 (m, 1H), 7.67-7.62 (m, 1H), 7.58-7.55 (m, 2H), 7.38 (d, J=8.0 Hz, 1H), 7.28-7.19 (m, 4H), 7.10 (d, J=7.6 Hz, 1H), 4.20-4.15 (m, 1H), 4.13-4.08 (m, 1H), 4.07-4.03 (m, 1H), 3.03-2.96 (m, 1H), 2.93-2.86 (m, 3H), 1.44 (d, J=6.8 Hz, 3H).

### Example 3: Synthesis of I-7

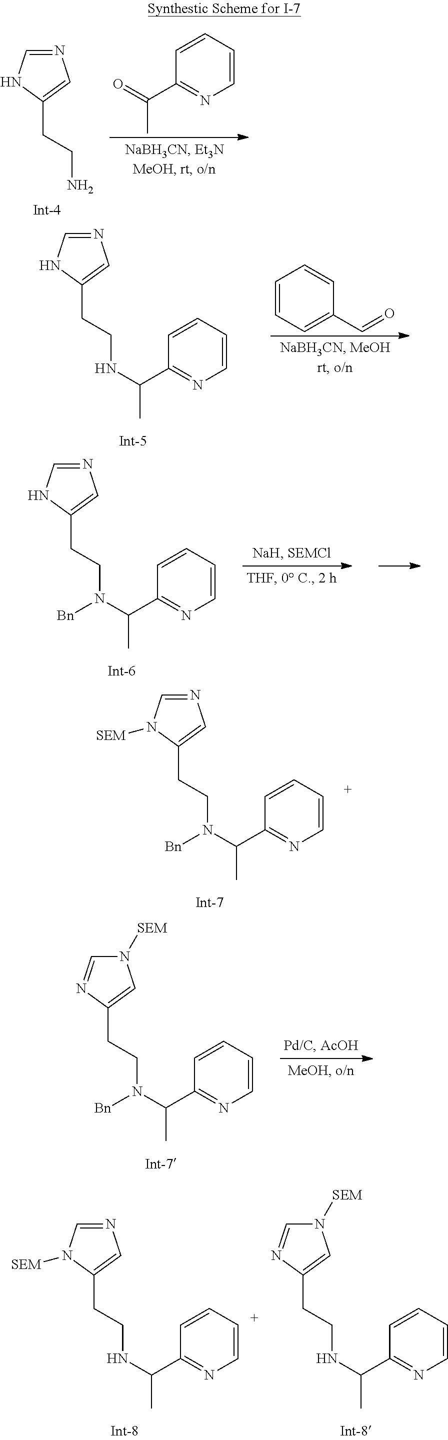

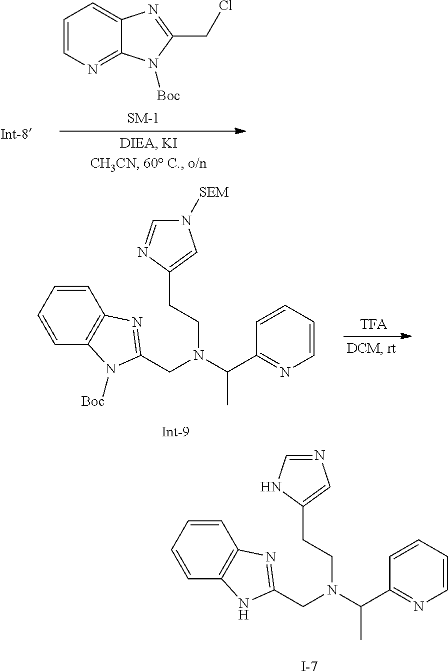

N-(2-(1H-imidazol-5-yl)ethyl)-1-(pyridin-2-yl)ethan-1-amine: Following general procedure A, Int-5 (2.80 g, yield: 95%) was obtained as yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 74%; Rt=1.13 min; MS Calcd.: 216.1; MS Found: 217.2 [M+H]⁺.

N-(2-(1H-imidazol-5-yl)ethyl)-N-benzyl-1-(pyridin-2-yl)ethan-1-amine: Following general procedure A, Int-6 (3.10 g, yield: 78%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 78%; Rt=1.71 min; MS Calcd.: 306.2; MS Found: 307.3 [M+H]⁺.

N-benzyl-1-(pyridin-2-yl)-N-(2-(1-((2-(trimethylsilyl)ethoxy)methyl)-1H-imidazol-5-yl)ethyl)ethan-1-amine; N-benzyl-1-(pyridin-2-yl)-N-(2-(1-((2-(trimethylsilyl)ethoxy)methyl)-1H-imidazol-4-yl)ethyl)ethan-1-amine: To a mixture of Int-6 (3.10 g, 10.13 mmol) in THE (100 mL) was added NaH (60%) (810 mg, 20.26 mmol) at 0° C., and the mixture was stirred at 0° C. for 30 min. After that the reaction mixture was added SEMCl (2.03 g, 12.16 mmol) and stirred at 0° C. for 1.5 h. After the reaction was completed, the mixture was quenched with H₂O and extracted by DCM (50 mL×3). The organic layers were washed with brine (20 mL×3), dried over Na₂SO₄, filtered and concentrated in vacuum to give a residue, which was purified by CC (DCM:MeOH=50:1) to give a mixture of Int-7 and Int-7′ (1.80 g, yield: 41%) as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 70%; Rt=2.13 min; MS Calcd.: 436.3; MS Found: 437.3 [M+H]⁺.

1-(Pyridin-2-yl)-N-(2-(1-((2-(trimethylsilyl)ethoxy)methyl)-1H-imidazol-5-yl)ethyl)ethan-1-amine; 1-(pyridin-2-yl)-N-(2-(1-((2-(trimethylsilyl)ethoxy)methyl)-1H-imidazol-4-yl)ethyl)ethan-1-amine: To a mixture of Int-7 and Int-7′ (1.00 g, 2.29 mmol), Pd/C (300 mg) in CH₃OH (46 mL) was added AcOH (6 drops), and the mixture was stirred at room temperature under H₂ atmosphere overnight. After the reaction was completed, the mixture was filtered, concentrated in vacuum, diluted with H₂O, neutralized by saturated NaHCO₃ aqueous solution to pH=8-9 and extracted by DCM (20 mL×3). The organic layers were dried over Na₂SO₄, filtered and concentrated in vacuum to give a residue, which was purified by CC (DCM:MeOH=50:1) to give a mixture of Int-8 and Int-8′ (200 mg, yield: 25%) as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 82%; Rt=1.72 min; MS Calcd.: 346.2; MS Found: 347.3 [M+H]*.

Tert-butyl 2-(((1-(pyridin-2-yl)ethyl)(2-(1-((2-(trimethylsilyl)ethoxy)methyl)-1H-imidazol-4-yl)ethyl)amino)methyl)-1H-benzo[d]imidazole-1-carboxylate: Following general procedure G, Int-9 (100 mg, yield: 60%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 85%; Rt=2.37 min; MS Calcd.: 576.3; MS Found: 577.4[M+H]*.

N-((1H-benzo[d]imidazol-2-yl)methyl)-N-(2-(1H-imidazol-5-yl)ethyl)-1-(pyridin-2-yl)ethan-1-amine: Following general procedure H, I-7 (23 mg, yield: 38%) was obtained as a white solid. LCMS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min). Purity: >99%, Rt=1.13 min; MS Calcd.: 346.2; MS Found: 347.2[M+H]*. HPLC (Agilent HPLC 1200; Column: L-column2 ODS (150 mm*4.6 mm*5.0 μm); Column Temperature: 40° C.; Flow Rate: 1.0 mL/min; Mobile Phase: from 95% [water+0.1% TFA] and 5% [CH₃CN+0.1% TFA] to 0% [water+0.1% TFA] and 100% [CH₃CN+0.1% TFA] in 10 min, then under this condition for 5 min, finally changed to 95% [water+0.1% TFA] and 5% [CH₃CN+0.1% TFA] in 0.1 min and under this condition for 5 min). Purity: 98%; Rt=4.47 min. ¹H NMR (400 MHz, CD₃OD): δ 8.53 (dd, J=4.8, 0.8 Hz, 1H), 7.80-7.74 (m, 1H), 7.56-7.50 (m, 4H), 7.31-7.27 (m, 1H), 7.24-7.21 (m, 2H), 6.66 (s, 1H), 4.17-4.11 (m, 2H), 4.01 (d, J=15.6 Hz, 1H), 2.94-2.88 (m, 1H), 2.80-2.69 (m, 3H), 1.48 (d, J=6.8 Hz, 3H).

### Example 4: Synthesis of I-12

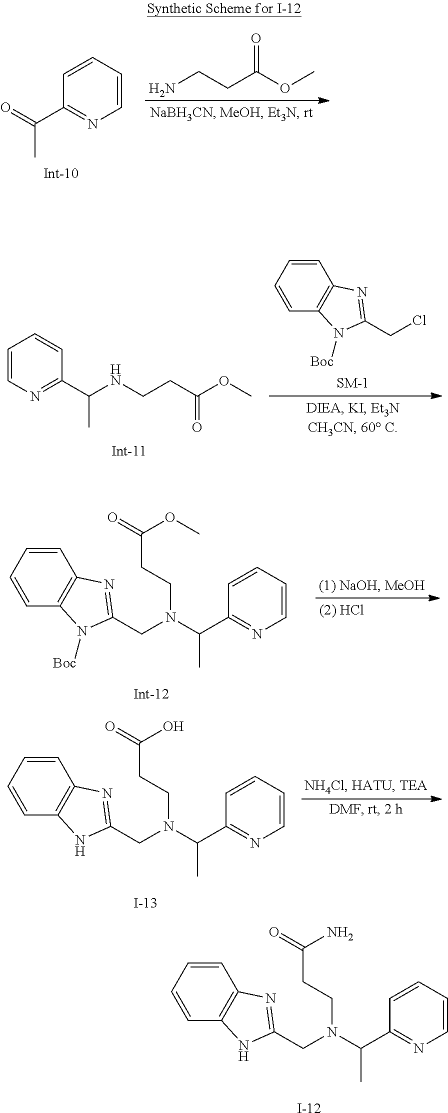

Methyl 3-((1-(pyridin-2-yl)ethyl)amino)propanoate: Following general procedure A, Int-11 (1.00 g, yield 67%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 91%; Rt=1.28 min; MS Calcd.: 208.1; MS Found: 209.1 [M+H]⁺.

Tert-butyl 2-(((3-methoxy-3-oxopropyl)(1-(pyridin-2-yl)ethyl)amino)methyl)-1H-benzo[d]imidazole-1-carboxylate: Following general procedure G, Int-12(330 mg, yield 52%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 90%; Rt=1.97 min; MS Calcd.: 438.2; MS Found: 439.2 [M+H]*.

3-(((1H-benzo[d]imidazol-2-yl)methyl)(1-(pyridin-2-yl)ethyl)amino)propanoic acid: Following general procedure I, Int-13 was obtained as a white solid, which was used in the next step directly. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 97%; Rt=1.19 min; MS Calcd.: 324.2; MS Found: 325.2[M+H]*.

3-(((1H-benzo[d]imidazol-2-yl)methyl)(1-(pyridin-2-yl)ethyl)amino)propanamide: Following general procedure D, I-12 (18.2 mg, yield 7.5%) was obtained as a yellow solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 96%; Rt=1.36 min; MS Calcd.: 323.2; MS Found: 324.2[M+H]*. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: 98%; Rt=6.13 min. ¹H NMR (400 MHz, CD₃OD): δ 8.55-8.53 (m, 1H), 7.82-7.77 (m, 1H), 7.58-7.53 (m, 3H), 7.32-7.21 (m, 3H), 4.14-4.08 (m, 2H), 3.95 (d, J=16.0 Hz, 1H), 3.03-2.97 (m, 1H), 2.88-2.83 (m, 1H), 2.44 (t, J=7.2 Hz, 2H), 1.49 (d, J=6.8 Hz, 3H).

### Example 5: Synthesis of I-14

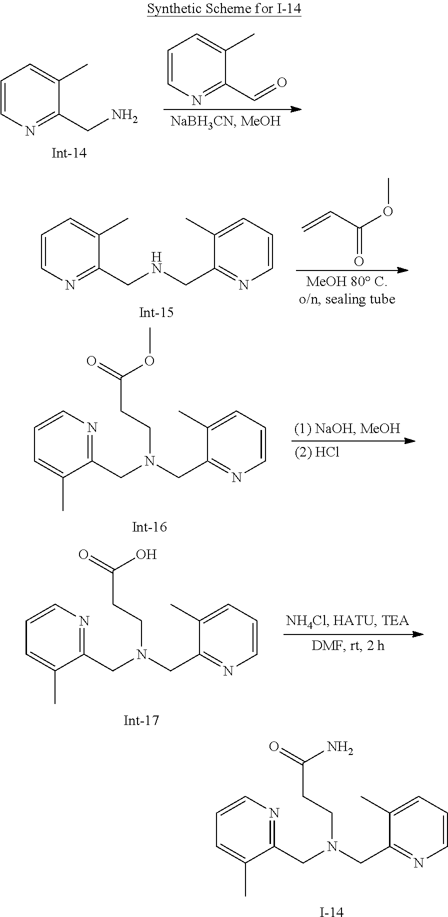

Bis((3-methylpyridin-2-yl)methyl)amine: Following general procedure A, Int-15 (300 mg, yield 32%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 74%; Rt=1.46 min; MS Calcd.: 227.1; MS Found: 228.1 [M+H]*.

Methyl 3-(bis((3-methylpyridin-2-yl)methyl)amino)propanoate: Following general procedure B, Int-16 (228 mg, yield 55%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 97%; Rt=1.62 min; MS Calcd.: 313.2; MS Found: 314.2 [M+H]*.

3-(Bis((3-methylpyridin-2-yl)methyl)amino)propanoic acid: Following general procedure C, Int-17 was obtained as a white solid, which was used in the next step directly. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 96%; Rt=1.21 min; MS Calcd.: 299.2; MS Found: 300.3[M+H]*.

3-(Bis((3-methylpyridin-2-yl)methyl)amino)propanamide: Following general procedure D, I-14 (51.4 mg, yield 23%) was obtained as a white solid. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: >99%; Rt=1.45 min; MS Calcd.: 298.2; MS Found: 299.3 [M+H]*. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: >99%; Rt=6.32 min. ¹H NMR (400 MHz, CD₃OD): δ 8.30-8.28 (m, 2H), 7.57 (d, J=7.2 Hz, 2H), 7.24 (dd, J=7.6, 4.8 Hz, 2H), 3.80 (s, 4H), 2.86 (t, J=7.2 Hz, 2H), 2.47 (m, J=7.2 Hz, 2H), 2.20 (s, 6H).

### Example 6: Synthesis of I-21

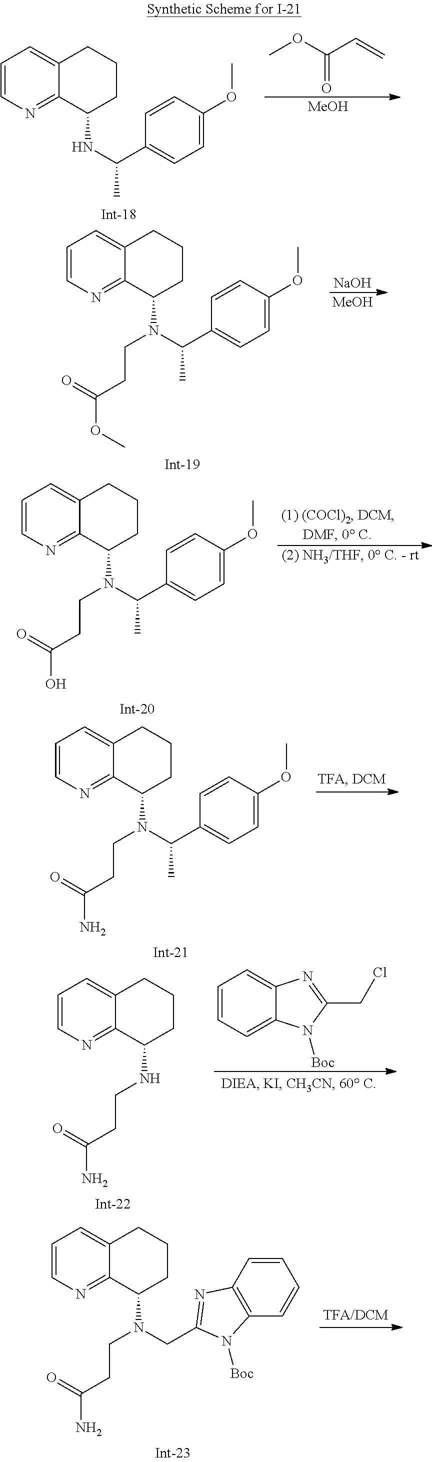

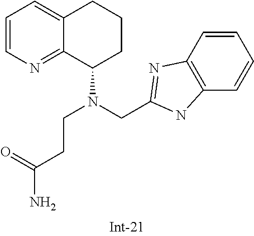 [SMILES: NC(=O)CCN(Cc1nc2ccccc2[nH]1)[C@H]1CCCc2cccnc21]

Methyl 3-(((S)-1-(4-methoxyphenyl)ethyl)((S)-5,6,7,8-tetrahydroquinolin-8-yl)amino)propanoate: Following general procedure B, Int-19 (1.93 g, yield: 34%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 89%; Rt=2.04 min; MS Calcd.: 368.2; MS Found: 369.3 [M+H]*.

3-(((S)-1-(4-Methoxyphenyl)ethyl)((S)-5,6,7,8-tetrahydroquinolin-8-yl)amino)propanoic acid: Following general procedure C, Int-20 (1.93 g, crude) was obtained as a white solid, which was used in the next step directly. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 91.39%; Rt=1.35 min; MS Calcd.: 354.2; MS Found: 355.2 [M+H]⁺

3-(((S)-1-(4-Methoxyphenyl)ethyl)((S)-5,6,7,8-tetrahydroquinolin-8-yl)amino)propanamide: To a mixture of Int-20 (1.85 g, 5.24 mmol), DMF (5 drops) in DCM (27 mL) was added oxalyl chloride (2.00 g, 15.72 mmol) dropwise at 0° C., and the mixture was stirred at 0° C. for 1 h. Then the reaction mixture was concentrated in vacuum at 10° C. to give a residue, which was added to a solution of NH₃/THF (5 M, 5 mL) in DCM (5 mL) at 0° C. Then the mixture was stirred at room temperature for 30 min, quenched with H₂O, concentrated in vacuum and extracted by DCM (10 mL×3). The combined organic phases were concentrated in vacuum to give a residue, which was purified by CC (DCM:MeOH=50:1) to give Int-21 (870 mg, yield 47%) as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 74.65%; Rt=1.92 min; MS Calcd.: 353.2; MS Found: 354.3 [M+H]⁺.

(S)-3-((5,6,7,8-Tetrahydroquinolin-8-yl)amino)propanamide: Following General procedure F, Int-22 (394 mg, yield: 73%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 94.36%; Rt=1.23 min; MS Calcd.: 219.1; MS Found: 220.2 [M+H]⁺.

Tert-butyl (S)-2-(((3-amino-3-oxopropyl)(5,6,7,8-tetrahydroquinolin-8-yl)amino)methyl)-1H-benzo[d]imidazole-1-carboxylate: Following general procedure G, Int-23 (80 mg, yield: 50%) was obtained as a yellow solid. LCMS (Agilent LCMS 1200-6110, Column: Waters X-Bridge C18 (50 mm*4.6 mm*3.5 μm); Column Temperature: 40° C.; Flow Rate: 2.0 mL/min; Mobile Phase: from 95% [water+0.05% TFA] and 5% [CH₃CN+0.05% TFA] to 0% [water+0.05% TFA] and 100% [CH₃CN+0.05% TFA] in 1.6 min, then under this condition for 1.4 min, finally changed to 95% [water+0.05% TFA] and 5% [CH₃CN+0.05% TFA] in 0.05 min and under this condition for 0.7 min). Purity: 73.58%; Rt=1.61 min; MS Calcd.: 449.2; MS Found: 450.3 [M+H]*.

(S)-3-(((1H-benzo[d]imidazol-2-yl)methyl)(5,6,7,8-tetrahydroquinolin-8-yl)amino)propanamide: Following general procedure H, I-21 (10 mg, yield: 16%) was obtained as a yellow solid. LCMS (Agilent HPLC 1200, Column: L-column2 ODS (150 mm*4.6 mm*5.0 m); Column Temperature: 40° C.; Flow Rate: 1.5 mL/min; Mobile Phase: from 90% [(total 10 mM AcONH₄) H₂O/MeCN=900/100 (v/v)] and 10% [(total 10 mM AcONH₄) H₂O/MeCN=100/900 (v/v)] to 15% [total 10 mM AcONH₄) H₂O/MeCN=900/100 (v/v)] and 85% [(total 10 mM AcONH₄) H₂O/MeCN=100/900 (v/v)] in 5 min, then under this condition for 10 min, finally changed to 90% [(total 10 mM AcONH₄) H₂O/MeCN=900/100 (v/v)] and 10% [(total 10 mM AcONH₄) H₂O/MeCN=100/900 (v/v)] in 0.1 min and under this condition for 5 min). Purity: 91.90%; Rt=1.46 min; MS Calcd.: 349.4; MS Found: 350.2[M+H]*. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: 92.61%; Rt=6.69 min. ¹H NMR (400 MHz, CD₃OD): δ 8.50 (d, J=4.0 Hz, 1H), 7.55-7.52 (m, 3H), 7.24-7.18 (m, 3H), 4.14-4.05 (m, 3H), 2.95-2.82 (m, 3H), 2.77-2.72 (m, 1H), 2.32 (t, J=7.2 Hz, 2H), 2.27-2.20 (m, 1H), 2.08-1.99 (m, 1H), 1.98-1.88 (m, 1H), 1.74-1.65 (m, 1H).

### Example 7: Synthesis of I-27

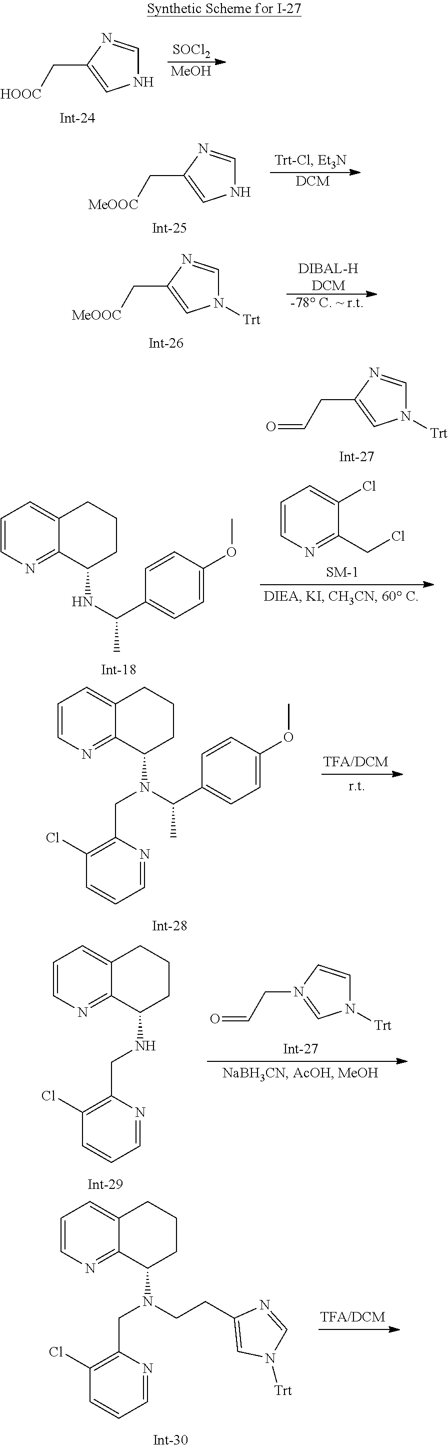

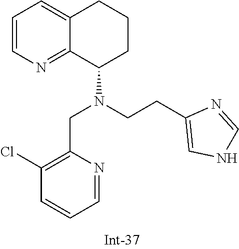 [SMILES: Clc1cccnc1CN(CCc1c[nH]cn1)[C@H]1CCCc2cccnc21]

Methyl 2-(1-trityl-1H-imidazol-4-yl)acetate: A solution of Int-24 (5.0 g, 30.9 mmol) and SOCl₂ (7.3 g, 61.7 mmol) in CH₃OH (120 mL) was stirred at 70° C. overnight. After completion of the reaction indicated by LCMS, the mixture was concentrated in vacuum, quenched by water, basified by K₂CO₃ to pH >7 and extracted with DCM (80 mL×3). The organic layers were dried over Na₂SO₄, filtered and concentrated in vacuum. Then DCM (100 mL) was added to the crude, followed by adding Et₃N (3750 mg, 37.0 mmol) and trityl chloride (10324 mg, 37.0 mmol). The mixture was stirred at room temperature overnight. After completion of the reaction indicated by LCMS, the mixture was concentrated in vacuum, quenched by water and then extracted with DCM (70 mL×3). The organic layer were dried over Na₂SO₄, filtered and concentrated in vacuum to give a residue, which was purified by column chromatography to obtain Int-26 (6.5 g, Yield: 55%) as a white solid. ¹H NMR (400 MHz, CDCl₃): δ 7.37 (d, J=1.2 Hz, 1H), 7.34-7.32 (m, 9H), 7.16-7.12 (m, 6H), 7.78-7.77 (m, 1H), 3.70 (s, 3H), 3.62 (s, 2H).

2-(1-Trityl-1H-imidazol-4-yl)acetaldehyde: To a mixture of Int-26 (4.0 g, 10.5 mmol) in DCM (55 mL) was added DIBAL-H (1M, 10.5 mL, 10.5 mmol) dropwise at −78° C. and the mixture was stirred at for 1.5 h, followed by adding more DIBAL-H (1M, 5.3 mL, 5.3 mmol) and stirred for 2.5 h. After Int-26 was consumed as indicated by TLC, the reaction mixture was quenched by adding MeOH (20 mL) dropwise at −78° C., warmed up slowly to room temperature, filtered and concentrated under reduced pressure to give Int-27 (3.3 g, yield 89%) as a colorless oil, which was used in the next step directly without purification.

(S)—N-((3-chloropyridin-2-yl)methyl)-N—((S)-1-(4-methoxyphenyl)ethyl)-5,6,7,8-tetrahydroquinolin-8-amine: Following general procedure G, Int-28 (240 mg, yield: 50%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 91.60%; Rt=2.41 min; MS Calcd.: 407.2; MS Found: 408.2 [M+H]*.

(S)—N-((3-chloropyridin-2-yl)methyl)-5,6,7,8-tetrahydroquinolin-8-amine: Following general procedure F, Int-29 (150 mg, yield: 93%) was obtained as a yellow oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 96.11%; Rt=1.94 min; MS Calcd.: 273.1; MS Found: 274.2 [M+H]*.

(S)—N-((3-chloropyridin-2-yl)methyl)-N-(2-(1-trityl-1H-imidazol-4-yl)ethyl)-5,6,7,8-tetrahydroquinolin-8-amine: Following general procedure A, Int-30 (150 mg, yield: 44%) was obtained as a brown oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 90.89%; Rt=2.80 min; MS Calcd.: 609.3; MS Found: 610.3 [M+H]*.

(S)—N-(2-(1H-imidazol-4-yl)ethyl)-N-((3-chloropyridin-2-yl)methyl)-5,6,7,8-tetrahydroquinolin-8-amine: Following general procedure E, I-27 (14 mg, yield: 15%) was obtained as a colorless oil. LCMS (Agilent LCMS 1200-6120, Column: Waters X-Bridge C18 (50 mm*4.6 mm*3.5 μm); Column Temperature: 40° C.; Flow Rate: 2.0 mL/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: >99.99%; Rt=2.23 min; MS Calcd.: 367.2; MS Found: 368.2 [M+H]*. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: >99.99%; Rt=10.70 min. ¹H NMR (400 MHz, CD₃OD): δ 8.39 (d, J=4.0 Hz, 1H), 8.30 (dd, J=4.8, 1.6 Hz, 1H), 7.61 (dd, J=8.0, 1.2 Hz, 1H), 7.54 (s, 1H), 7.42 (d, J=7.6 Hz, 1H), 7.16-7.08 (m, 2H), 6.53 (s, 1H), 4.21 (t, J=7.6 Hz, 1H), 4.14 (d, J=13.6 Hz, 1H), 3.97 (d, J=13.2 Hz, 1H), 3.01-2.97 (m, 2H), 2.89-2.80 (m, 1H), 2.77-2.58 (m, 3H), 2.15-2.09 (m, 2H), 2.07-1.99 (m, 1H), 1.73-1.66 (m, 1H)

### Example 8: Synthesis of 1-31

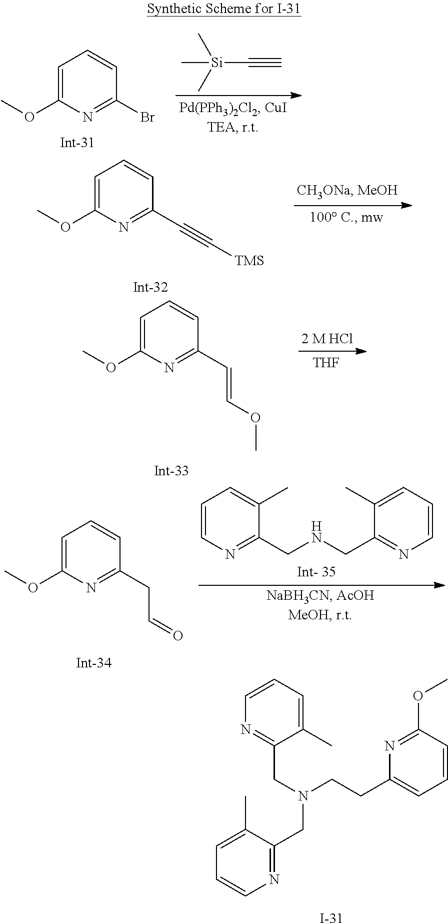

2-Methoxy-6-((trimethylsilyl)ethynyl)pyridine: A mixture of Int-31 (1.5 g, 7.98 mmol), Pd(PPh₃)₂C2 (280 mg, 0.40 mmol) and CuI (152 mg, 0.80 mmol) in TEA (20 mL) was added ethynyltrimethylsilane (862 mg, 8.79 mmol) at room temperature under nitrogen atmosphere, and the solution was stirred at room temperature overnight. After completion of the reaction indicated by LCMS, the mixture was filtered and the filtrate was concentrated in vacuum, and the residue was purified by column chromatography to give Int-32 (1.0 g, 61%) as colorless liquid. LCMS (Agilent LCMS 1200-6120, Column: Halo C18 (30 mm*4.6 mm*2.7 μm); Column Temperature: 40° C.; Flow Rate: 3.0 ml/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] in 0.01 min and under this condition for 0.2 min). Purity: 90.62%; Rt=0.94 min; MS Calcd.: 205.1; MS Found: 206.3 [M+H]*.

(E)-2-Methoxy-6-(2-methoxyvinyl)pyridine: A mixture of Int-32 (1.0 g, 4.87 mmol) and MeONa (525 mg, 9.74 mmol) in MeOH (10 mL) was stirred at 100° C. for 1.5 h under microwave irritation. After completion of the reaction indicated by LCMS, the mixture was concentrated in vacuum, and the residue was suspended in water and DCM. The organic layer was washed with brine, dried over Na₂SO₄ and filtered. The filtrate was concentrated in vacuum to give Int-33 (600 mg, 75%) as light brown liquid. LCMS (Agilent LCMS 1200-6120, Column: Halo C18 (30 mm*4.6 mm*2.7 μm); Column Temperature: 40° C.; Flow Rate: 3.0 ml/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] in 0.01 min and under this condition for 0.2 min). Purity: 67.39%; Rt=0.65 min; MS Calcd.: 165.1; MS Found: 166.3 [M+H]⁺.

2-(6-Methoxypyridin-2-yl)acetaldehyde: A mixture of Int-33 (500 mg, 3.03 mmol) and 2 M HCl aqueous (12 mL, 24.21 mmol) in THE (5 mL) was stirred at 70° C. for 2 h in sealed tube. After completion of the reaction indicated by LCMS, the mixture was concentrated in vacuum, and the residue was quenched by saturated NaHCO₃ solution, extracted with DCM. The organic layer was washed with brine, dried over Na₂SO₄ and filtered. The filtrate was concentrated in vacuum to give the crude Int-35 (400 mg, 87%) as light brown liquid. LCMS (Agilent LCMS 1200-6120, Column: Waters X-Bridge C18 (50 mm*4.6 mm*3.5 μm); Column Temperature: 40° C.; Flow Rate: 2.0 ml/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 9.98%; Rt=1.20 min; MS Calcd.: 151.1; MS Found: 152.3 [M+H]*, 170.2 [M+H₂O+H]*.

2-(6-Methoxypyridin-2-yl)-N,N-bis((3-methylpyridin-2-yl)methyl)ethan-1-amine: Following General Procedure A, I-31(14 mg, yield: 3%) was obtained as a light yellow oil. LCMS (Agilent LCMS 1200-6120, Column: Waters X-Bridge C18 (50 mm*4.6 mm*3.5 μm); Column Temperature: 40° C.; Flow Rate: 2.0 ml/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 98.56%; Rt=1.84 min; MS Calcd.: 362.2; MS Found: 363.2 [M+H]*. HPLC (AgilentHPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: 99.68%; Rt=10.03 min; MS Calcd.: 362.2; MS Found: 363.2 [M+H]*. ¹H NMR (400 MHz, CDCl₃) δ: 8.38 (dd, J=4.8, 1.2 Hz, 2H), 7.38-7.33 (m, 3H), 7.10 (dd, J=7.2, 4.8 Hz, 2H), 6.48 (t, J=7.2 Hz, 2H), 3.85 (s, 4H), 3.81 (s, 3H), 3.01-2.98 (m, 2H), 2.88-2.85 (m, 2H), 2.10 (s, 6H).

### Example 9: Synthesis of I-43

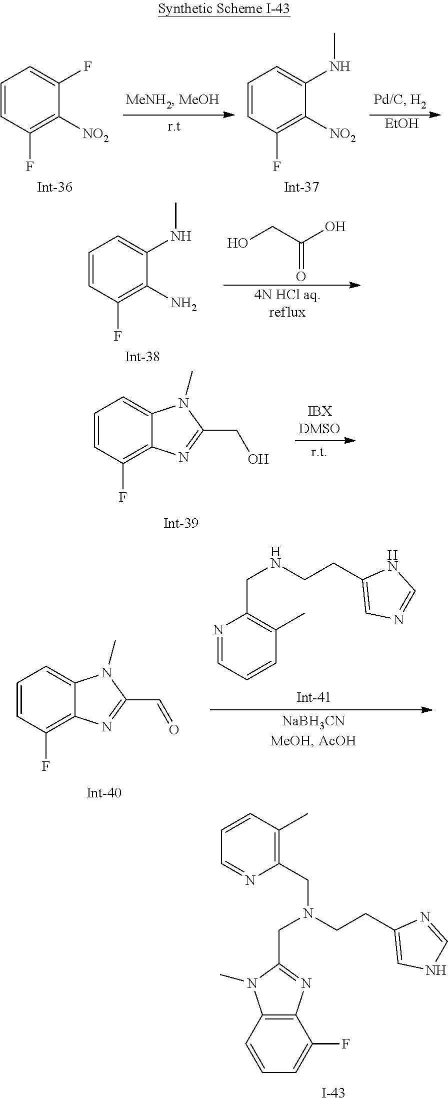

3-Fluoro-N-methyl-2-nitroaniline: To a solution of Int-36 (1.0 g, 6.3 mmol) in MeOH (5 mL) was added 33% methyl amine (1.3 g, 13.8 mmol) in MeOH at 0° C., and the mixture was stirred at the same temperature for 0.5 h, then at room temperature for 3 h. After completion of the reaction indicated by LCMS, the reaction solution was poured into ice water, and precipitated crystals were collected by filtration and washed with water. The residue was dissolved in DCM, dried over Na₂SO₄ and filtered. The filtrate was concentrated in vacuum to give Int-37 (800 mg, 75%). LCMS (Agilent LCMS 1200-6120, Column: Halo C18 (30 mm*4.6 mm*2.7 m); Column Temperature: 40° C.; Flow Rate: 3.0 ml/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] in 0.01 min and under this condition for 0.2 min). Purity: 100%; Rt=0.74 min; MS Calcd.: 171.1; MS Found: 170.1 [M+H]*.

3-Fluoro-N-1-methylbenzene-1,2-diamine: A mixture of Int-37 (800 mg, 4.70 mmol) and 10% Pd/C (80 mg) in EtOH (50 mL) was stirred at room temperature for 4 h under H₂ atmosphere. After completion of the reaction indicated by LCMS, the mixture was filtered through Celite and the filtrate was concentrated in vacuum to give crude product Int-38 (500 mg, 76%) as yellow oil which was used for the next step without further purification. LCMS (Agilent LCMS 1200-6120, Column: Halo C18 (30 mm*4.6 mm*2.7 μm); Column Temperature: 40° C.; Flow Rate: 3.0 ml/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] in 0.01 min and under this condition for 0.2 min). Purity: 55.89%; Rt=0.39 min; MS Calcd.: 140.1; MS Found: 141.4 [M+H]*.

(4-Fluoro-1-methyl-1H-benzo[d]imidazol-2-yl)methanol: To a solution of Int-38 (500 mg, 3.57 mmol) in 4 M HCl aq (10 mL) was added 2-hydroxyacetic acid (299 mg, 3.93 mmol) at room temperature, and the mixture was stirred overnight at 90° C. After completion of the reaction indicated by LCMS, the solution was cooled to room temperature, adjusted to pH=8 with solid NaHCO₃, and extracted with EA. The organic phase was dried over Na₂SO₄, filtered and concentrated in vacuum to give crude product Int-39 (300 mg, 47%) as yellow oil which was used for the next step without further purification. LCMS (Agilent LCMS 1200-6120, Column: Halo C18 (30 mm*4.6 mm*2.7 μm); Column Temperature: 40° C.; Flow Rate: 3.0 ml/min; Mobile Phase: from 95% [water+0.05% TFA] and 5% [CH₃CN+0.05% TFA] to 0% [water+0.05% TFA] and 100% [CH₃CN+0.05% TFA] in 0.8 min, then under this condition for 0.4 min, finally changed to 95% [water+0.05% TFA] and 5% [CH₃CN+0.05% TFA] in 0.01 min and under this condition for 0.2 min). Purity: 100%, Rt=0.37 min; MS Calcd.: 180.1; MS Found: 181.3 [M+H]*.

4-Fluoro-1-methyl-1H-benzo[d]imidazole-2-carbaldehyde: A mixture of Int-39 (300 mg, 1.67 mmol) and IBX (932 mg, 3.33 mmol) in DMSO (10 mL) was stirred at room temperature overnight. After completion of the reaction indicated by LCMS, the mixture was quenched by water, and extracted with DCM. The organic layer was washed with brine, dried over Na₂SO₄ and filtered. The filtrate was concentrated in vacuum to give crude product Int-40 (250 mg, 84%) as brown oil which was used for next step without further purification. LCMS (Agilent LCMS 1200-6120, Column: Waters X-Bridge C18 (50 mm*4.6 mm*3.5 μm); Column Temperature: 40° C.; Flow Rate: 2.0 ml/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 59.72%; Rt=1.52 min; MS Calcd.: 178.1; MS Found: 179.2 [M+H]*, 197.2 [M+H₂O+H]*.

N-((4-fluoro-1-methyl-1H-benzo[d]imidazol-2-yl)methyl)-2-(1H-imidazol-4-yl)-N-((3-methylpyridin-2-yl)methyl)ethan-1-amine: Following General Procedure A, I-43 (29 mg, yield: 17%) was obtained as light-yellow oil. LCMS (Agilent LCMS 1200-6120, Column: Waters X-Bridge C18 (50 mm*4.6 mm*3.5 μm); Column Temperature: 40° C.; Flow Rate: 2.0 ml/min; Mobile Phase: from 95% [water+10 mM NH₄HCO₃] and 5% [CH₃CN] to 0% [water+10 mM NH₄HCO₃] and 100% [CH₃CN] in 1.6 min, then under this condition for 1.4 min, finally changed to 95% [water+10 mM NH₄HCO₃] and 5% [CH₃CN] in 0.1 min and under this condition for 0.7 min). Purity: 94.57%; Rt=1.49 min; MS Calcd.: 378.2; MS Found: 379.2 [M+H]*. HPLC (Agilent HPLC 1200, Column: Waters X-Bridge C18 (150 mm*4.6 mm*3.5 μm); Column Temperature: 40° C.; Flow Rate: 1.0 mL/min; Mobile Phase: from 95% [water+10 mM NH₄HCO₃] and 5% [CH₃CN] to 0% [water+10 mM NH₄HCO₃] and 100% [CH₃CN] in 10 min, then under this condition for 5 min, finally changed to 95% [water+10 mM NH₄HCO₃] and 5% [CH₃CN] in 0.1 min and under this condition for 5 min). Purity: 99.37%; Rt=7.57 min; MS Calcd.: 378.2; MS Found: 379.2 [M+H]⁺. ¹H NMR (400 MHz, CDCl₃) δ: 8.34 (d, J=3.6 Hz, 1H), 7.68 (s, 1H), 7.22 (d, J=6.8 Hz, 1H), 7.13-7.09 (m, 1H), 6.95-6.91 (m, 3H), 6.90-6.83 (m, 1H), 3.98 (s, 2H), 3.94 (s, 2H), 3.43 (s, 3H), 3.03-2.98 (m, 4H), 2.28 (s, 3H).

### Example 10: Synthesis of Additional Compounds

Additional exemplary compounds were prepared following methods substantially similar to those described above and herein. Data for these compounds are provided below in Table 2.

TABLE 2
Characterization Data for Additional Exemplary Compounds

| Compound | | | Rt (Min) | Rt (Min) | ¹H NMR |
|---|---|---|---|---|---|
| No. | Chemical Structure | M + 1 | (LCMS) | (HPLC) | (400 MHz) |
| I-3 | 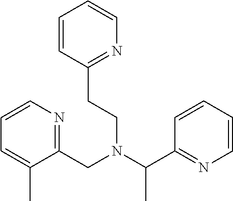 [SMILES: Cc1cccnc1CN(CCc1ccccn1)C(C)c1ccccn1] | 333.3 | 1.78 | 8.16 | ¹H NMR (400 MHz, CD₃OD) δ: 8.51-8.48 (m, 1H), 8.29-8.25 (m, 2H), 7.78-7.73 (m, 1H), 7.65- 7.60 (m, 1H), 7.49 (d, J = 6.8 Hz, 1H), 7.43 (d, J = 7.6 Hz, 1H), 7.31-7.27 (m, 1H), 7.24-7.20 (m, 1H), 7.20-7.15 (m, 1H), 6.99 (d, J = 8.0 Hz, 1H), 4.18 (dd, J = 13.6, 6.8 Hz, 1H), 3.88 (dd, J = |
| | | | | | 20.4, 12.8 Hz, 2H), 3.03- |
| | | | | | 2.95 (m, 1H), 2.89-2.74 |
| | | | | | (m, 3H), 2.12 (s, 3H), |
| | | | | | 1.53 (d, J = 7.2 Hz, 3H). |
| I-4 | 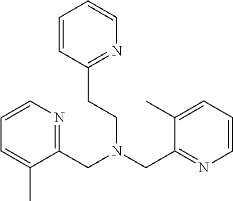 [SMILES: Cc1cccnc1CN(CCc1ccccn1)Cc1ncccc1C] | 333.3 | 1.76 | 8.08 | ¹H NMR (400 MHz, CD₃OD) δ: 8.33-8.29 (m, 3H), 7.65-7.60 (m, 1H), 7.56-7.54 (m, 2H), 7.26 (dd, J = 7.6, 5.2 Hz, 2H), 7.21-7.16 (m, 1H), 7.02 (d, J = 7.6 Hz, 1H), 3.84 (s, 4H), 2.99-2.90 (m, 4H), 2.10 (s, 6H). |
| I-5 | 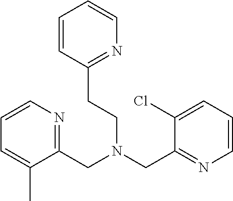 [SMILES: Cc1cccnc1CN(CCc1ccccn1)Cc1ncccc1Cl] | 353.3 | 1.83 | 8.44 | ¹H NMR (400 MHz, CDCl₃): δ 8.45 (dd, J = 4.8, 1.6 Hz, 1H), 8.42- 8.40 (m, 1H), 8.36-8.34 (m, 1H), 7.61 (dd, J = 8.0, 1.2 Hz, 1H), 7.49- 7.45 (m, 1H), 7.33 (d, J = 6.8 Hz, 1H), 7.16-7.13 (m, 1H), 7.08-6.98 (m, 3H), 4.01 (s, 2H), 3.94 (s, 2H), 3.03 (s, 4H), 2.09 (s, 3H). |
| I-6 | 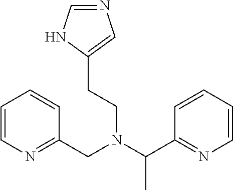 [SMILES: CC(c1ccccn1)N(CCc1cnc[nH]1)Cc1ccccn1] | 308.2 | 1.4 | 6.48 | ¹H NMR (400 MHz, CD₃OD): δ 8.49 (d, J = 4.8 Hz, 1H), 8.43 (d, J = 4.4 Hz, 1H), 7.77-7.71 (m, 2H), 7.56 (s, 1H), 7.47 (d, J = 8.0 Hz, 2H), 7.28-7.24 (m, 2H), 6.69 (s, 1H), 4.14 (q, J = 6.8 Hz, 1H), 3.92-3.79 (m, 2H), 2.99-2.90 (m, 1H), 2.82-2.71 (m, 3H), 1.46 (d, J = 6.8 Hz, 3H). |
| I-8 | 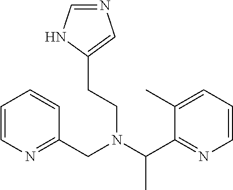 [SMILES: Cc1cccnc1C(C)N(CCc1cnc[nH]1)Cc1ccccn1] | 322.2 | 1.47 | 7.08 | ¹H NMR (400 MHz, CD₃OD): δ 8.49 (d, J = 4.8 Hz, 1H), 8.31 (d, J = 4.0 Hz, 1H), 7.75-7.71 (m, 1H), 7.54-7.51 (m, 2H), 7.40 (d, J = 8.0 Hz, 1H), 7.28-7.25 (m, 1H), 7.23-7.20 (m, 1H), 6.55 (s, 1H), 4.11 (q, J = 6.8 Hz, 1H), 3.95-3.87 (m, 2H), 2.93-2.87 (m, 1H), 2.77-2.70 (m, 1H), 2.61 |
| | | | | | (t, J = 7.2 Hz, 2H), 2.24 |
| | | | | | (s, 3H), 1.49 (d, J = |
| | | | | | 6.8 Hz, 3H). |
| I-9 | 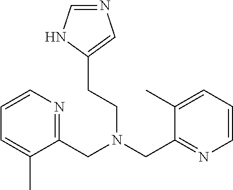 [SMILES: Cc1cccnc1CN(CCc1cnc[nH]1)Cc1ncccc1C] | 322.2 | 1.45 | 6.89 | ¹H NMR (400 MHz, CDCl₃): δ 8.31 (d, J = 3.6 Hz, 2H), 7.60 (s, 1H), 7.25 (d, J = 7.6 Hz, 2H), 7.01-6.98 (m, 2H), 6.74 (s, 1H), 3.84 (s, 4H), 2.93-2.85 (m, 4H), 2.12 (s, 6H). |
| I-10 | 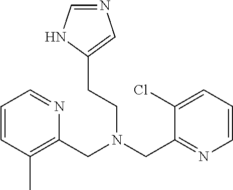 [SMILES: Cc1cccnc1CN(CCc1cnc[nH]1)Cc1ncccc1Cl] | 342.1 | 1.5 | 7.22 | ¹H NMR (400 MHz, CD₃OD): δ 8.45 (dd, J = 4.8, 1.6 Hz, 1H), 8.26 (d, J = 4.4 Hz, 1H), 7.78 (dd, J = 8.0, 1.2 Hz, 1H), 7.51-7.48 (m, 2H), 7.32- 7.29 (m, 1H), 7.21-7.18 (m, 1H), 6.59 (s, 1H), 3.96 (s, 2H), 3.89 (s, 2H), 2.90-2.86 (m, 2H), 2.82- 2.79 (m, 2H), 2.16 (s, 3H). |
| I-11 | 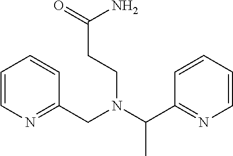 [SMILES: CC(c1ccccn1)N(CCC(N)=O)Cc1ccccn1] | 285.3 | 1.33 | 5.82 | ¹H NMR (400 MHz, CD₃OD): δ 8.50-8.48 (m, 1H), 8.43-8.41 (m, 1H), 7.82-7.77 (m, 2H), 7.64- 7.57 (m, 2H), 7.30-7.26 (m, 2H), 4.11 (dd, J = 13.6, 6.8 Hz, 1H), 3.89 (d, J = 15.6 Hz, 1H), 3.77 (d, J = 15.2 Hz, 1H), 3.05-2.98 (m, 1H), 2.84- |
| | | | | | 2.77 (m, 1H), 2.43-2.38 |
| | | | | | (m, 2H), 1.49 (d, J = |
| | | | | | 6.8 Hz, 3H) |
| I-13 | 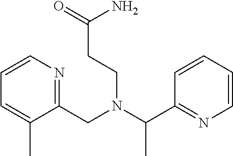 [SMILES: Cc1cccnc1CN(CCC(N)=O)C(C)c1ccccn1] | 299.3 | 1.42 | 6.31 | ¹H NMR (400 MHz, CD₃OD): δ 8.51-8.48 (m, 1H), 8.28-8.26 (m, 1H), 7.79-7.74 (m, 1H), 7.53 (t, J = 7.6 Hz, 2H), 7.30- 7.19 (m, 2H), 4.12 (dd, J = 13.6, 6.8 Hz, 1H), 3.85 (dd, J = 14.8, 13.2 Hz, 2H), 2.97-2.76 (m, 2H), 2.33-2.29 (m, 2H), 2.25 |
| | | | | | (s, 3H), 1.53 (d, J = |
| | | | | | 6.8 Hz, 3H). |
| I-15 | 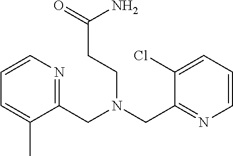 [SMILES: Cc1cccnc1CN(CCC(N)=O)Cc1ncccc1Cl] | 319.2 | 1.47 | 6.51 | ¹H NMR (400 MHz, CD₃OD): δ 8.44 (d, J = 4.0 Hz, 1H), 8.27 (d, J = 4.4 Hz, 1H), 7.80 (d, J = 8.0 Hz, 1H), 7.54 (d, J = 7.2 Hz, 1H), 7.32 (dd, J = 8.0, 4.8 Hz, 1H), 7.23 (dd, 7.2, 5.2 Hz, 1H), 3.93 (s, 2H), 3.87 (s, 2H), 2.92 (t, J = 6.8 Hz, 2H), |
| | | | | | 2.49 (t, J = 7.2 Hz, 2H), |
| | | | | | 2.22 (s, 3H). |
| I-16 | 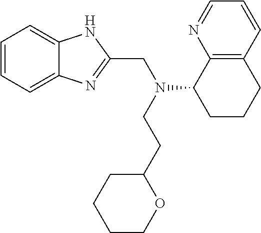 [SMILES: c1cnc2c(c1)CCC[C@@H]2N(CCC1CCCCO1)Cc1nc2ccccc2[nH]1] | 391.3 | 1.35 | 5.83 | ¹H NMR (400 MHz, CD₃OD): δ 8.57-8.53 (m, 1H), 7.59-7.53 (m, 3H), 7.28-7.20 (m, 3H), 4.15- 3.97 (m, 3H), 3.75-3.69 (m, 1H), 3.31-3.24 (m, 2H), 2.89-2.84 (m, 1H), 2.80-2.75 (m, 1H), 2.67- 2.53 (m, 2H), 2.25-2.21 (m, 1H), 2.08-1.96 (m, 2H), 1.73-1.65 (m, 2H), 1.48-1.32 (m, 5H), 1.25- 1.14 (m, 1H), 1.04-0.98 (m, 1H). |
| I-17 | 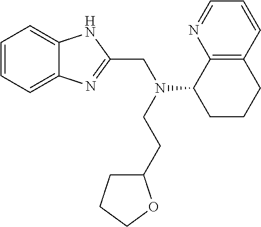 [SMILES: c1cnc2c(c1)CCC[C@@H]2N(CCC1CCCO1)Cc1nc2ccccc2[nH]1] | 377.3 | 1.87 | 9.24 | ¹H NMR (400 MHz, CD₃OD): δ 8.50 (t, J = 5.2 Hz, 1H), 7.54-7.52 (m, 3H), 7.24-7.19 (m, 3H), 4.11-3.98 (m, 3H), 3.74-3.54 (m, 2H), 3.34- 3.31 (m, 1H), 2.85-2.81 (m, 1H), 2.74-2.67 (m, 1H), 2.61-2.56 (m, 2H), 2.22-2.20 (m, 1H), 2.04- 1.91 (m, 2H), 1.77-1.69 (m, 4H), 1.61-1.39 (m, 2H), 1.22-1.18 (m, 1H). |
| I-18 | 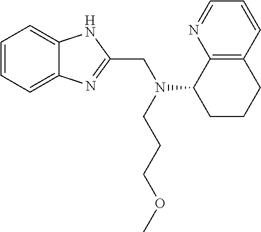 [SMILES: COCCCN(Cc1nc2ccccc2[nH]1)[C@H]1CCCc2cccnc21] | 351.3 | 1.81 | 8.94 | ¹H NMR (400 MHz, CD₃OD): δ 8.56 (d, J = 4.0 Hz, 1H), 7.60-7.57 (m, 3H), 7.29-7.23 (m, 3H), 4.16-4.03 (m, 3H), 3.31-3.27 (m, 2H), 3.16 (s, 3H), 2.90-2.86 (m, 1H), 2.80-2.77 (m, 1H), 2.65-2.56 (m, 2H), 2.27- 2.24 (m, 1H), 2.09-1.97 (m, 2H), 1.73-1.64 (m, 3H). |
| I-19 | 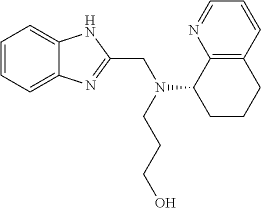 [SMILES: OCCCN(Cc1nc2ccccc2[nH]1)[C@H]1CCCc2cccnc21] | 337.2 | 1.79 | 7.42 | ¹H NMR (400 MHz, CD₃OD): δ 8.51 (d, J = 4.0 Hz, 1H), 7.58-7.53 (m, 3H), 7.26-7.20 (m, 3H), 4.15-4.01 (m, 3H), 3.48 (t, J = 6.0 Hz, 2H), 2.92-2.85 (m, 1H), 2.80- 2.69 (m, 2H), 2.63-2.55 (m, 1H), 2.30-2.24 (m, 1H), 2.11-2.05 (m, 1H), 2.02-1.92 (m, 1H), 1.76- 1.50 (m, 3H). |
| I-20 | 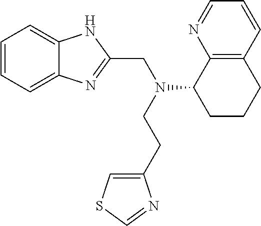 [SMILES: c1cnc2c(c1)CCC[C@@H]2N(CCc1cscn1)Cc1nc2ccccc2[nH]1] | 390.3 | 1.8 | 8.9 | ¹H NMR (400 MHz, CDCl₃): δ 8.63 (d, J = 2.0 Hz, 1H), 8.44 (d, J = 3.6 Hz, 1H), 7.57 (brs, 2H), 7.39 (d, J = 7.6 Hz, 1H), 7.22-7.18 (m, 2H), 7.13- 7.09 (m, 1H), 6.83 (d, J = 2.0 Hz, 1H), 4.23-4.12 (m, 2H), 4.06-4.02 (m, 1H), 3.14-3.02 (m, 2H), 2.92-2.78 (m, 3H), 2.71- 2.66 (m, 1H), 2.19-2.13 (m, 1H), 2.03-1.95 (m, 1H), 1.91-1.82 (m, 1H), 1.71-1.59 (m, 1H). |
| I-22 | 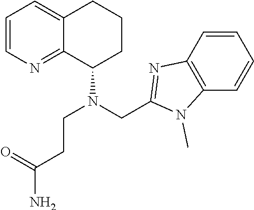 [SMILES: Cn1c(CN(CCC(N)=O)[C@H]2CCCc3cccnc32)nc2ccccc21] | 364.3 | 1.11 | 4.07 | ¹H NMR (400 MHz, CD₃OD): δ 8.37 (d, J = 4.0 Hz, 1H), 7.57 (d, J = 7.6 Hz, 1H), 7.44-7.37 (m, 2H), 7.30-7.20 (m, 2H), 7.06 (dd, J = 7.6, 4.8 Hz, 1H), 4.20 (d, J = 13.6 Hz, 1H), 4.14-4.06 (m, 2H), 3.91 (s, 3H), 3.04- 2.97 (m, 2H), 2.91-2.83 (m, 1H), 2.74-2.68 (m, 1H), 2.49-2.41 (m, 1H), 2.34-2.26 (m, 1H), 2.23- |
| | | | | | 2.06 (m, 3H), 1.74-1.69 |
| | | | | | (m, 1H). |
| I-23 | 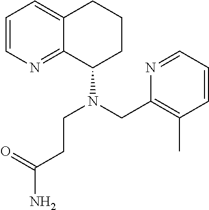 [SMILES: Cc1cccnc1CN(CCC(N)=O)[C@H]1CCCc2cccnc21] | 325.2 | 1.69 | 7.58 | ¹H NMR (400 MHz, CD₃OD): δ 8.36 (d, J = 4.0 Hz, 1H), 8.24 (d, J = 4.0 Hz, 1H), 7.53 (d, J = 7.6 Hz, 1H), 7.45 (d, J = 8.0 Hz, 1H), 7.18 (dd, J = 7.6, 4.8 Hz, 1H), 7.13 (dd, J = 7.6, 4.8 Hz, 1H), 4.06-4.00 (m, 2H), 3.91 (d, J = 12.8 Hz, 1H), 2.93- 2.80 (m, 3H), 2.74-2.68 (m, 1H), 2.38 (s, 4H), 2.21-2.02 (m, 4H), 1.73- |
| | | | | | 1.63 (m, 1H). |
| I-24 | 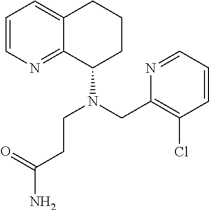 [SMILES: NC(=O)CCN(Cc1ncccc1Cl)[C@H]1CCCc2cccnc21] | 345.1 | 1.56 | 8.05 | ¹H NMR (400 MHz, CD₃OD): δ 8.39 (dd, J = 4.4, 1.2 Hz, 1H), 8.33 (d, J = 4.4 Hz, 1H), 7.74 (dd, J = 8.4, 1.6 Hz, 1H), 7.44 (d, J = 7.6 Hz, 1H), 7.25 (dd, J = 8.0, 4.8 Hz, 1H), 7.11 (dd, J = 7.6, 4.4 Hz, 1H), 4.19-4.15 (m, 2H), 4.01 (d, J = 14.0 Hz, 1H), 3.10-3.04 (m, 1H), 3.01- 2.94 (m, 1H), 2.88-2.79 (m, 1H), 2.74-2.67 (m, |
| | | | | | 1H), 2.44-2.27 (m, 2H), |
| | | | | | 2.14-1.98 (m, 3H), 1.72- |
| | | | | | 1.67 (m, 1H). |
| I-25 | 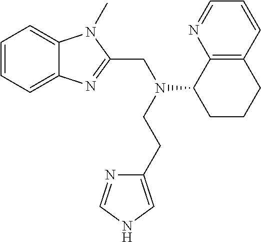 [SMILES: Cn1c(CN(CCc2c[nH]cn2)[C@H]2CCCc3cccnc32)nc2ccccc21] | 387.2 | 1.61 | 8.11 | ¹H NMR (400 MHz, CD₃OD): δ 8.42 (d, J = 4.4 Hz, 1H), 7.58 (s, 1H), 7.50 (d, J = 12 Hz, 1H), 7.30 (d, J = 7.6 Hz, 2H), 7.23-7.14 (m, 2H), 6.99 (dd, J = 7.6, 4.8 Hz, 1H), 6.59 (s, 1H), 4.27-4.22 (m, 1H), 4.11-4.01 (m, 2H), 3.68 (s, 3H), 3.06- 3.00 (m, 1H), 2.98-2.91 (m, 1H), 2.90-2.75 (m, 2H), 2.69-2.58 (m, 2H), 2.28-2.14 (m, 2H), 2.09- 2.05 (m, 1H), 1.72-1.69 |
| | | | | | (m, 1H). |
| I-26 | 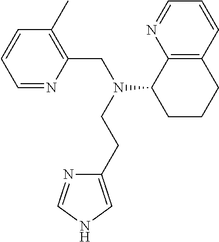 [SMILES: Cc1cccnc1CN(CCc1c[nH]cn1)[C@H]1CCCc2cccnc21] | 348.3 | 1.66 | 7.79 | ¹H NMR (400 MHz, CD₃OD): δ 8.45 (d, J = 4.0 Hz, 1H), 8.19 (dd, J = 4.8, 1.2 Hz, 1H), 7.56 (s, 1H), 7.44 (d, J = 7.2 Hz, 1H), 7.39 (d, J = 7.2 Hz, 1H), 7.16-7.07 (m, 2H), 6.52 (s, 1H), 4.17-4.13 (m, 1H), 3.98 (d, J = 12.8 Hz, 1H), 3.86 (d, J = 12.4 Hz, 1H), 2.92-2.81 (m, 3H), 2.72-2.66 (m, 2H), 2.54-2.48 (m, 1H), 2.19 (s, 3H), 2.17-2.10 (m, 3H), 1.52-1.23 (m, 1H). |
| I-28 | 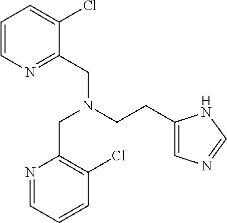 [SMILES: Clc1cccnc1CN(CCc1cnc[nH]1)Cc1ncccc1Cl] | 362.1 | 1.22 | 5.03 | ¹H NMR (400 MHz, CDCl₃) δ: 8.41 (d, J = 4.4 Hz, 2H), 7.92 (s, 1H), 7.56 (d, J = 8.0 Hz, 2H), 7.11 (dd, J = 8.0, 4.8 Hz, 2H), 6.76 (s, 1H), 5.77 (s, 1H), 4.06 (s, 4H), 3.02 (t, J = 5.2 Hz, 2H), 2.81 (t, J = 5.2 Hz, 2H). |
| I-29 | 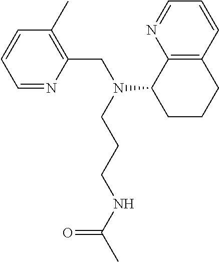 [SMILES: CC(=O)NCCCN(Cc1ncccc1C)[C@H]1CCCc2cccnc21] | 353.4 | 1.6 | 7.46 | ¹H NMR (400 MHz, CD₃OD): δ 8.39 (s, 1H), 8.24 (s, 1H), 7.47 (d, J = 4.8 Hz, 2H), 7.18 (s, 2H), 4.10 (s, 1H), 3.93-3.90 (m, 2H), 3.20-3.18 (m, 1H), 2.84-2.71 (m, 2H), 2.35 (s, 3H), 2.12 (brs, 3H), 1.90 (s, 3H), 1.70 (s, 3H), 1.52-1.47 (m, 2H). |
| I-30 | 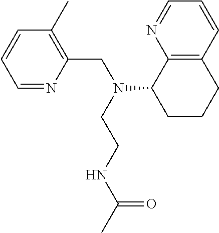 [SMILES: CC(=O)NCCN(Cc1ncccc1C)[C@H]1CCCc2cccnc21] | 339.2 | 1.1 | 4.54 | ¹H NMR (400 MHz, CD₃OD): δ 8.41 (d, J = 4.4 Hz, 1H), 8.24 (d, J = 4.4 Hz, 1H), 7.55 (d, J = 7.6 Hz, 1H), 7.47 (d, J = 7.6 Hz, 1H), 7.21-7.13 (m, 2H), 4.09-3.92 (m, 3H), 2.99-2.90 (m, 2H), 2.88-2.79 (m, 1H), 2.73- 2.65 (m, 3H), 2.47 (s, 3H), 2.31-2.27 (m, 1H), 2.06-2.00 (m, 1H), 1.98- 1.89 (m, 4H), 1.72-1.63 (m, 1H). |
| I-32 | 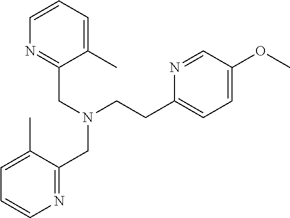 [SMILES: COc1ccc(CCN(Cc2ncccc2C)Cc2ncccc2C)nc1] | 363.3 | 1.13 | 4.52 | ¹H NMR (400 MHz, CDCl₃) δ: 8.38 (d, J = 4.8 Hz, 2H), 8.13 (d, J = 3.2 Hz, 1H), 7.37 (d, J = 7.6 Hz, 2H), 7.10 (dd, J = 7.6, 5.2 Hz, 2H), 7.01 (dd, J = 8.4, 3.2 Hz, 1H), 7.01 (dd, J = 8.4, 3.2 Hz, 1H), 6.84 (d, J = 8.8 Hz, 1H), 3.85 (s, 4H), 3.81 (s, 3H), 2.95 (s, 4H), 2.08 (s, 6H). |
| I-33 | 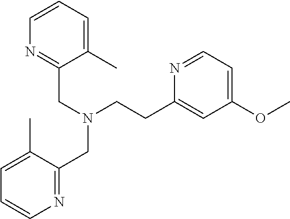 [SMILES: COc1ccnc(CCN(Cc2ncccc2C)Cc2ncccc2C)c1] | 363.3 | 1.8 | 8.57 | ¹H NMR (400 MHz, CDCl₃) δ: 8.30 (dd, J = 4.4, 0.8 Hz, 2H), 8.16 (d, J = 6.0 Hz, 1H), 7.29 (d, J = 7.2 Hz, 2H), 7.01 (dd, J = 7.6, 4.8 Hz, 2H), 6.51 (dd, J = 6.0, 2.8 Hz, 1H), 6.43 (d, J = 2.4 Hz, 1H), 3.78 (s, 4H), 3.70 (s, 3H), 2.89 (s, 4H), 2.01 (s, 6H). |
| I-34 | 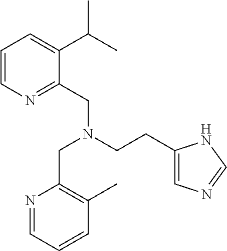 [SMILES: Cc1cccnc1CN(CCc1cnc[nH]1)Cc1ncccc1C(C)C] | 350.4 | 1.19 | 8.16 | ¹H NMR (400 MHz, DMSO-d₆) δ: 12.08- 11.64 (m, 1H), 8.34 (d, J = 3.6 Hz, 1H), 8.29 (dd, J = 4.4, 1.2 Hz, 1H), 7.61 (dd, J = 7.6, 1.2 Hz, 1H), 7.56 (d, J = 7.2 Hz, 1H), 7.44 (s, 1H), 7.25-7.20 (m, 2H), 6.49 (br, 1H), 3.77 (s, 2H), 3.70 (s, 2H), 2.86-2.78 (m, 1H), 2.71 (br, 4H), 2.19 (s, 3H), 0.90 (d, J = 6.8 Hz, 6H). |
| I-35 | 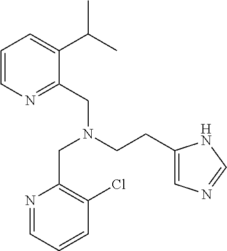 [SMILES: CC(C)c1cccnc1CN(CCc1cnc[nH]1)Cc1ncccc1Cl] | 370.3 | 1.3 | 5.24 | ¹H NMR (400 MHz, DMSO-d₆) δ: 12.00- 11.62 (m, 1H), 8.50 (dd, J = 4.4, 1.2 Hz, 1H), 8.28 (dd, 4.4, 1.2 Hz, 1H), 7.91 (dd, J = 8.0, 1.2 Hz, 1H), 7.62-7.60 (m, 1H), 7.43 (d, J = 0.8 Hz, 1H), 7.39-7.36 (m, 1H), 7.25- 7.21 (m, 1H), 6.62-6.43 (m, 1H), 3.88 (s, 2H), 3.81 (s, 2H), 2.96-2.89 (m, 1H), 2.75-2.70 (m, 4H), 0.95 (d, J = 7.2 Hz, 6H). |
| I-36 | 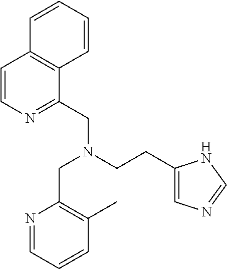 [SMILES: Cc1cccnc1CN(CCc1cnc[nH]1)Cc1nccc2ccccc12] | 358.3 | 1.17 | 7.88 | ¹H NMR (400 MHz, CDCl₃) 13.74 (br, 1H), 8.32 (d, J = 5.6 Hz, 1H), 8.02-8.00 (m, 2H), 7.66- 7.55 (m, 3H), 7.46-7.40 (m, 2H), 7.17 (d, J = 7.6 Hz, 1H), 6.82-6.79 (m, 2H), 4.31 (s, 2H), 3.92 (s, 2H), 3.07-3.04 (m, 2H), 2.96-2.93 (m, 2H), 2.17 (s, 3H). |
| I-37 | 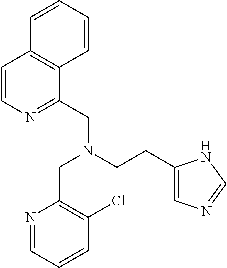 [SMILES: Clc1cccnc1CN(CCc1cnc[nH]1)Cc1nccc2ccccc12] | 378.1 | 1.27 | 4.74 | ¹H NMR (400 MHz, CDCl₃) δ: 8.27 (d, J = 5.6 Hz, 1H), 8.00 (dd, J = 4.4, 1.2, 1H), 7.94 (d, J = 8.4 Hz, 1H), 7.60-7.58 (m, 1H), 7.54-7.50 (m, 1H), 7.39-7.32 (m, 4H), 6.82 (dd, J = 8.0, 4.8 Hz, 1H), 6.67 (s, 1H), 4.30 (s, 2H), 4.03 (s, 2H), 3.01- 2.98 (m, 2H), 2.83-2.80 (m, 2H). |
| I-38 | 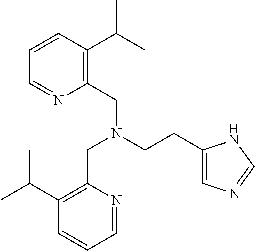 [SMILES: CC(C)c1cccnc1CN(CCc1cnc[nH]1)Cc1ncccc1C(C)C] | 378.3 | 1.29 | 9.06 | ¹H NMR (400 MHz, DMSO-d₆) δ: 12.14- 11.63 (m, 1H), 8.33 (dd, J = 4.8, 1.2 Hz, 2H), 7.66 (dd, J = 8.0, 1.2 Hz, 2H), 7.44 (s, 1H), 7.26 (dd, J = 7.6, 4.8 Hz, 2H), 6.47 (br, 1H), 3.78 (s, 4H), 3.08- 3.01 (m, 2H), 2.72 (br, 4H), 0.99 (d, J = 6.8 Hz, 12H). |
| I-39 | 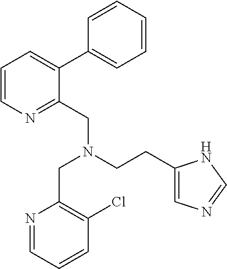 [SMILES: Clc1cccnc1CN(CCc1cnc[nH]1)Cc1ncccc1-c1ccccc1] | 404.2 | 1.35 | 5.27 | ¹H NMR (400 MHz, DMSO-d₆) δ: 11.92 (br, 1H), 8.56 (d, J = 3.2 Hz, 1H), 8.36 (d, J = 3.6 Hz, 1H), 7.78 (d, J = 8.0 Hz, 1H), 7.62 (d, J = 6.4 Hz, 1H), 7.43-7.36 (m, 4H), 7.32-7.25 (m, 4H), 6.51 (s, 1H), 3.85 (s, 2H), 3.82 (s, 2H), 2.77-2.74 (m, 2H), 2.55-2.51 (m, 2H). |
| I-40 | 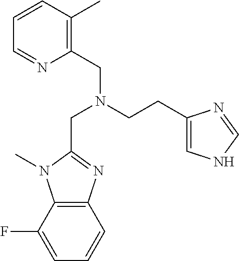 [SMILES: Cc1cccnc1CN(CCc1c[nH]cn1)Cc1nc2cccc(F)c2n1C] | 379.2 | 1.53 | 7.89 | ¹H NMR (400 MHz, CDCl₃) 13.77 (br, 1H), 8.27 (d, J = 4.0 Hz, 1H), 7.56 (s, 1H), 7.29 (d, J = 8.0 Hz, 1H), 7.08 (d, J = 6.4 Hz, 1H), 7.00-6.95 (m, 2H), 6.88-6.85 (m, 1H), 6.78-6.73 (m, 2H), 3.85-3.84 (m, 4H), 3.54 (s, 3H), 2.94-2.87 (m, 4H), 2.16 (s, 3H). |
| I-41 | 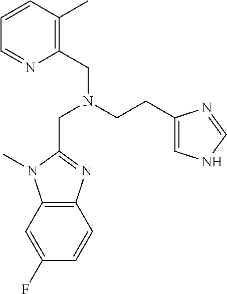 [SMILES: Cc1cccnc1CN(CCc1c[nH]cn1)Cc1nc2ccc(F)cc2n1C] | 379.3 | 1.51 | 7.42 | ¹H NMR (400 MHz, CDCl₃): δ 8.29 (dd, J = 4.8, 1.2 Hz, 1H), 7.56 (s, 1H), 7.56 (t, J = 7.6 Hz, 1H), 7.47 (dd, J = 8.8, 4.8 Hz, 1H), 7.12 (d, J = 7.6 Hz, 1H), 6.89-6.82 (m, 2H), 6.70 (s, 1H), 6.68 (d, J = 1.2 Hz, 1H), 3.85 (s, 2H), 3.84 (s, 2H), 3.28 (s, 3H), 2.92-2.87 (m, 4H), 2.18 (s, 3H). |
| I-42 | 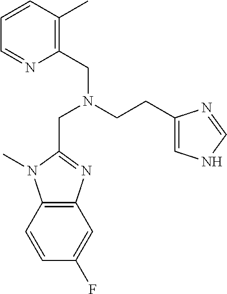 [SMILES: Cc1cccnc1CN(CCc1c[nH]cn1)Cc1nc2cc(F)ccc2n1C] | 379.2 | 1.5 | 7.58 | ¹H NMR (400 MHz, CDCl₃) 8.27 (dd, J = 4.8, 1.2, 1H), 7.57 (s, 1H), 7.22-7.20 (m, 1H), 7.13 (d, J = 7.6 Hz, 1H), 6.94- 6.90 (m, 1H), 6.88-6.83 (m, 2H), 6.73 (s, 1H), 3.86-3.84 (m, 4H), 3.31 (s, 3H), 2.93-2.87 (m, 4H), 2.18 (s, 3H). |
| I-44 | 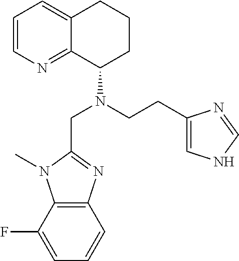 [SMILES: Cn1c(CN(CCc2c[nH]cn2)[C@H]2CCCc3cccnc32)nc2cccc(F)c21] | 405.3 | 1.85 | 8.68 | ¹H NMR (400 MHz, CD₃OD): δ 8.44 (d, J = 3.6 Hz, 1H), 7.62 (s, 1H), 7.31-7.26 (m, 2H), 7.12-7.06 (m, 1H), 7.01- 6.98 (dd, J = 7.6, 4.4 Hz, 1H), 6.93-6.87 (m, 1H), 6.62 (s, 1H), 4.29-4.24 (m, 1H), 4.06 (q, J = 13.6 Hz, 2H), 3.85 (s, 3H), 3.08-3.03 (m, 1H), 3.00- 2.95 (m, 1H), 2.87-2.79 (m, 2H), 2.72-2.66 (m, 2H), 2.30-2.23 (m, 1H), 2.21-2.10 (m, 1H), 2.10- 2.05 (m, 1H), 1.75-1.68 |
| | | | | | (m, 1H). |
| I-45 | 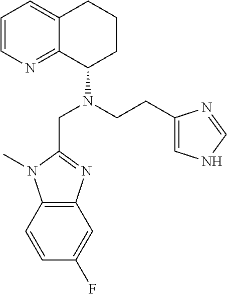 [SMILES: Cn1c(CN(CCc2c[nH]cn2)[C@H]2CCCc3cccnc32)nc2cc(F)ccc21] | 405.3 | 1.75 | 8.41 | ¹H NMR (400 MHz, CD₃OD): δ 8.41 (d, J = 3.6 Hz, 1H), 7.59 (s, 1H), 7.33-7.27 (m, 2H), 7.17 (dd, J = 8.4, 2.4 Hz, 1H), 7.03-6.97 (m, 2H), 6.61 (s, 1H), 4.24 (dd, J = 8.8, 6.8 Hz, 1H), 4.01 (q, J = 14.0 Hz, 2H), 3.68 (s, 3H), 3.08-3.01 (m, 1H), 2.99-2.75 (m, 3H), 2.70-2.61 (m, 2H), 2.28-2.14 (m, 2H), 2.10- 2.05 (m, 1H), 1.74-1.68 (m, 1H). |
| I-46 | 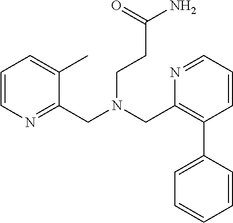 [SMILES: Cc1cccnc1CN(CCC(N)=O)Cc1ncccc1-c1ccccc1] | 361.2 | 1.79 | 7.87 | ¹H NMR (400 MHz, CD₃OD): δ 8.52 (dd, J = 4.8, 1.6 Hz, 1H), 8.17 (d, J = 4.0 Hz, 1H), 7.63 (dd, J = 8.0, 2.0 Hz, 1H), 7.45 (d, J = 7.6 Hz, 1H), 7.41- 7.38 (m, 1H), 7.36-7.32 (m, 3H), 7.30-7.25 (m, 2H), 7.18-7.15 (m, 1H), 3.83 (s, 2H), 3.66 (s, 2H), 2.69 (t, J = 6.8 Hz, 2H), 2.19 (t, J = 6.8 Hz, 2H), 2.01 (s, 3H). |
| I-47 | 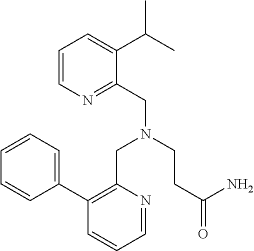 [SMILES: CC(C)c1cccnc1CN(CCC(N)=O)Cc1ncccc1-c1ccccc1] | 389.3 | 1.9 | 8.71 | ¹H NMR (400 MHz, CD₃OD): δ 8.56 (dd, J = 4.8, 1.6 Hz, 1H), 8.19 (dd, J = 4.4, 1.6 Hz, 1H), 7.71 (dd, J = 8.0, 2.0 Hz, 1H), 7.66 (dd, J = 8.0, 1.6 Hz, 1H), 7.45-7.35 (m, 6H), 7.26-7.24 (m, 1H), 3.83 (s, 2H), 3.71 (s, 2H), 2.93-2.85 (m, 1H), 2.71 (t, J = 6.8 Hz, 2H), 2.15 (t, J = 6.8 Hz, 2H), 0.98 (d, J = 6.4 Hz, 6H). |
| I-48 | 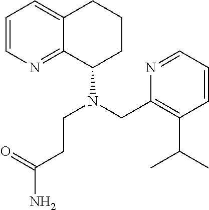 [SMILES: CC(C)c1cccnc1CN(CCC(N)=O)[C@H]1CCCc2cccnc21] | 353.2 | 1.81 | 8.28 | ¹H NMR (400 MHz, CD₃OD): δ 8.40 (d, J = 4.4 Hz, 1H), 8.26 (d, J = 4.0 Hz, 1H), 7.73 (d, J = 7.6 Hz, 1H), 7.50 (d, J = 7.2 Hz, 1H), 7.30-7.26 (m, 1H), 7.17 (dd, J = 7.6, 4.8 Hz, 1H), 4.07 (d, J = 12.8 Hz, 1H), 3.99 (t, J = 6.8 Hz, 1H), 3.84 (d, J = 12.4 Hz, 1H), 3.49-3.41 (m, 1H), 2.90-2.82 (m, 3H), 2.77-2.70 (m, 1H), |
| | | | | | 2.46-2.38 (m, 1H), 2.18- |
| | | | | | 2.03 (m, 4H), 1.73-1.6.3 |
| | | | | | (m, 1H), 1.18 (d, J = |
| | | | | | 6.8 Hz, 3H), 1.06 (d, J = |
| | | | | | 6.8 Hz, 3H). |
| I-49 | 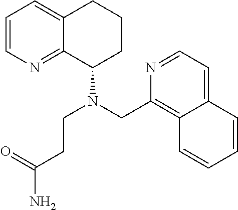 [SMILES: NC(=O)CCN(Cc1nccc2ccccc12)[C@H]1CCCc2cccnc21] | 361.3 | 1.81 | 8.3 | ¹H NMR (400 MHz, CD₃OD): δ 8.51 (d, J = 8.4 Hz, 1H), 8.32 (d, J = 3.2 Hz, 1H), 8.29 (d, J = 5.6 Hz, 1H), 7.85 (d, J = 8.0 Hz, 1H), 7.74-7.69 (m, 1H), 7.66 (d, J = 5.6 Hz, 1H), 7.63-7.58 (m, 1H), 7.43 (d, J = 7.6 Hz, 1H), 7.09 (dd, J = 7.6, 4.4 Hz, 1H), 4.46 (d, J = 12.4 Hz, 1H), 4.27 (d, J = 12.4 Hz, 1H), 4.15 (t, J = |
| | | | | | 7.6 Hz, 1H), 3.02-2.96 (m, |
| | | | | | 2H), 2.85-2.80 (m, 1H), |
| | | | | | 2.74-2.67 (m, 1H), 2.43- |
| | | | | | 2.37 (m, 1H), 2.22-2.15 |
| | | | | | (m, 3H), 2.09-2.04 (m, |
| | | | | | 1H), 1.71-1.65 (m, 1H). |
| I-50 | 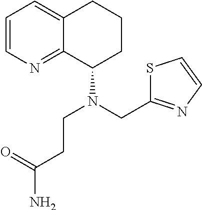 [SMILES: NC(=O)CCN(Cc1nccs1)[C@H]1CCCc2cccnc21] | 317 | 1.63 | 7.29 | ¹H NMR (400 MHz, CD₃OD): δ 8.39 (dd, J = 4.0, 0.8 Hz, 1H), 7.62 (d, J = 3.6 Hz, 1H), 7.47- 7.45 (m, 2H), 7.16-7.13 (m, 1H), 4.29 (d, J = 16.4 Hz, 1H), 4.12-4.08 (m, 1H), 4.02 (d, J = 16.4 Hz, 1H), 3.06-2.96 (m, 2H), 2.84-2.73 (m, 2H), 2.48- 2.37 (m, 2H), 2.17-2.14 (m, 1H), 2.05-2.01 (m, 1H), 1.94-1.90 (m, 1H), |
| | | | | | 1.72-1.70 (m, 1H). |
| I-51 | 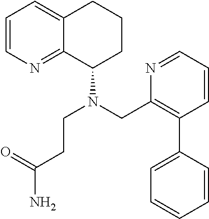 [SMILES: NC(=O)CCN(Cc1ncccc1-c1ccccc1)[C@H]1CCCc2cccnc21] | 387.2 | 1.94 | 5.57 | ¹H NMR (400 MHz, CD₃OD): δ 8.48 (dd, J = 4.8, 1.6 Hz, 1H), 8.21 (d, J = 4.0 Hz, 1H), 7.61- 7.57 (m, 1H), 7.41-7.30 (m, 7H), 7.50 (dd, J = 8.0, 4.8 Hz. 1H), 4.11- 3.96 (m, 2H), 3.81 (dd, J = 8.8, 5.6 Hz, 1H), 2.94- 2.86 (m, 1H), 2.83-2.76 (m, 1H), 2.66-2.56 (m, 2H), 2.19-2.08 (m, 2H), 1.85-1.72 (m, 2H), 1.67- |
| | | | | | 1.57 (m, 1H), 1.54-1.44 |
| | | | | | (m, 1H). |
| I-52 | 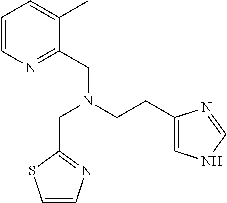 [SMILES: Cc1cccnc1CN(CCc1c[nH]cn1)Cc1nccs1] | 314.2 | 1.45 | 6.84 | ¹H NMR (400 MHz, CDCl₃): δ 8.51 (d, J = 7.6 Hz, 1H), 7.66 (s, 1H), 7.63 (d, J = 3.2 Hz, 1H), 7.47 (dd, J = 6.8, 0.8 Hz, 1H), 7.17-7.14 (m, 2H), 6.77 (s, 1H), 3.99 (s, 2H), 3.95 (s, 2H), 2.95-2.90 (m, 4H), 2.32 (s, 3H). |
| I-53 | 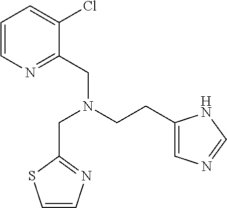 [SMILES: Clc1cccnc1CN(CCc1cnc[nH]1)Cc1nccs1] | 334.2 | 1.53 | 7.07 | ¹H NMR (400 MHz, CDCl₃): δ 8.60 (d, J = 6.8 Hz, 1H), 7.71 (s, 1H), 7.66 (s, 2H), 7.29 (s, 1H), 7.26-7.20 (m, 1H), 6.79 (s, 1H), 4.15 (s, 2H), 4.10 (s, 2H), 2.99-2.91 (m, 4H). |
| I-54 | 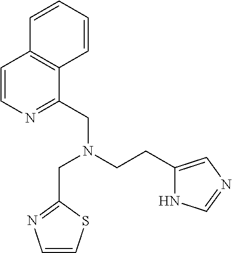 [SMILES: c1ccc2c(CN(CCc3cnc[nH]3)Cc3nccs3)nccc2c1] | 350.3 | 1.65 | 7.38 | ¹H NMR (400 MHz, CDCl₃): δ 8.46 (d, J = 5.6 Hz, 1H), 8.11 (d, J = 8.4 Hz, 1H), 7.44 (d, J = 8.4 Hz, 1H), 7.65-7.61 (m, 1H), 7.55-7.50 (m, 4H), 7.19 (d, J = 4.4 Hz, 1H), 6.71 (s, 1H), 4.41 (s, 2H), 4.04 (s, 2H), 2.98 (t, J = 6.0 Hz, 2H), 2.87 (t, J = 6.4 Hz, 2H). |
| I-55 | 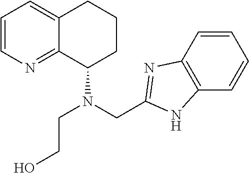 [SMILES: OCCN(Cc1nc2ccccc2[nH]1)[C@H]1CCCc2cccnc21] | 323.3 | 1.73 | 7.9 | ¹H NMR (400 MHz, DMSO-d₆): δ 12.36 (s, 1H), 8.51-8.39 (m, 1H), 7.63-7.49 (m, 3H), 7.23- 7.09 (m, 3H), 4.66-4.62 (m, 1H), 4.30-4.18 (m, 1H), 4.06-3.91 (m, 2H), 2.91-2.64 (m, 4H), 2.16- 1.60 (m, 5H) |

### Example 11: REGA Screenin2 Assay

### Intracellular CXCL-12-Induced Calcium Mobilization Assay

Intracellular calcium mobilization induced by chemokines orchemokine-derived peptides were evaluated using a calcium responsive fluorescent probe and a FLIPR system. The CXCR-4 transfected U87 cell line (U87.CXCR4) cells were seeded in gelatine-coated black-wall 96-well plates at 20,000 cells per well and incubated for 12 hours. Cells were then loaded with the fluorescent calcium probe Fluo-2 acetoxymethyl at 4 μM final concentration in assay buffer (Hanks' balanced salt solution with 20 mM HEPES buffer and 0.2% bovine serum albumin, pH 7.4) for 45 min at 37′C. The intracellular calcium mobilization induced by the CXCL-12 (25-50 ng/mL) was then measured at 37° C. by monitoring the fluorescence as a function of time in all the wells simultaneously using a fluorometric imaging plate reader (FLIPR Tetra, Molecular Devices). The test compounds were added 15 minutes before the addition of CXCL-12 and monitored to see if compounds induced signals by themselves (agonistic properties).

### Chemokine (CXCL12-AF647) Binding Inhibition Assay

Jurkat cells expressing CXCR4 were washed once with assay buffer (Hanks' balanced salt solution with 20 mM HEPES buffer and 0.2% bovine serum albumin, pH 7.4) and then incubated for 15 min at room temperature with the test compounds diluted in assay buffer at dose-dependent concentrations. Subsequently, CXCL12-AF647 (25 ng/mL) was added to the compound-incubated cells. The cells were incubated for 30 min at room temperature. Thereafter, the cells were washed twice in assay buffer, fixed in 1% paraformaldehyde in PBS, and analyzed on the FL4 channel of a FACSCalibur flow cytometer equipped with a 635-nm red diode laser (Becton Dickinson, San Jose, Calif., USA).

The percentages of inhibition of CXCL12-AF647 binding were calculated according to the formula: [1−((MFI−MFI_{NC})/(MFI_{PC}−MFI_{NC}))]×100 where MFI is the mean fluorescence intensity of the cells incubated with CXCL12-AF647 in the presence of the inhibitor, MFI_{NC} is the mean fluorescence intensity measured in the negative control (i.e., autofluorescence of unlabeled cells), and MFI_{PC} is the mean fluorescence intensity of the positive control (i.e., cells exposed to CXCL12-AF647 alone).

### Results of Assays

Table 3 shows the activity of selected compounds of this invention in the assays described above. The compound numbers correspond to the compound numbers in Table 1. Compounds having an activity designated as “A” provided an IC₅₀ of 0.01 to 100 nM; compounds having an activity designated as “B” provided an IC₅₀ of >100 nm to <1 μM; and compounds having an activity designated as “C” provided an IC₅₀ of 1 μM or greater.

TABLE 3
Inhibition of Ca²⁺ Signaling and Inhibition of CXCL12 Binding

| | IC₅₀ CXCL-12 | IC₅₀ CXCL-12 |
|---|---|---|
| Compound | Ca2+ flux | binding Jurkat |
| # | U87.CXCR4+ (nM) | (nM) |
| I-1 | C | A |
| I-2 | A | A |
| I-3 | B | A |
| I-4 | B | A |
| I-5 | B | A |
| I-6 | C | A |
| I-7 | C | A |
| I-8 | C | A |
| I-9 | A | A |
| I-10 | B | A |
| I-11 | C | B |
| I-12 | C | B |
| I-13 | C | A |
| I-14 | C | B |
| I-15 | C | B |
| I-16 | C | B |
| I-17 | C | A |
| I-18 | B | A |
| I-19 | B | A |
| I-20 | A | A |
| I-21 | B | A |
| I-22 | B | A |
| I-23 | C | A |
| I-24 | C | A |
| I-25 | A | A |
| I-26 | C | A |
| I-27 | C | A |
| I-28 | B | A |
| I-29 | C | A |
| I-30 | B | A |
| I-31 | A | A |
| I-32 | C | A |
| I-33 | C | A |
| I-34 | B | A |
| I-35 | B | A |
| I-36 | A | A |
| I-37 | A | A |
| I-38 | C | B |
| I-39 | C | A |
| I-40 | C | A |
| I-41 | B | A |
| I-42 | C | A |
| I-43 | A | A |
| I-44 | A | A |
| I-45 | C | A |
| I-46 | C | A |
| I-47 | C | B |
| I-48 | C | A |
| I-49 | B | A |
| I-50 | C | C |
| I-51 | C | A |
| I-52 | C | B |
| I-53 | C | B |
| I-54 | C | B |
| I-55 | C | B |
| I-56 | C | A |
| I-57 | B | A |
| I-58 | C | B |
| I-59 | C | B |

### Example 12: Caco-2 Permeability Assay

### Assay Procedure

The goal of this assay was to evaluate the intestinal absorption potential of drug candidates using Caco-2 cell lines.

Experimental Procedure
1. Pre-warm Pre-warm HBSS Buffer in 37° C. water bath
2. Sonicate Take compounds from −20° C., sonicate for a few minutes (no less than 1 minute)
3. Solution preparation

### Donor Solution Buffer

For A-to-B Direction:
HBSS buffer with 0.3% DMSO and 5 μM LY: add 150 μL DMSO and 50 μL LY (5 mM) into 50 ml HBSS buffer (pH 7.4).
HBSS buffer with 0.1% DMSO and 5 μM LY: add 50 μL DMSO and 50 μL LY (5 mM) into 50 mL HBSS buffer (pH 7.4).

For B-to-A Direction:
HBSS buffer with 0.3% DMSO: add 150 μL DMSO into 50 ml HBSS buffer (pH 7.4). HBSS buffer with 0.1% DMSO: add 50 μL DMSO into 50 ml HBSS buffer (pH 7.4).

Receiver Solution Buffer:

For A-to-B Direction:
Prepare HBSS buffer with 0.4% DMSO: add 200 μL DMSO into 50 ml HBSS buffer (pH 7.4).

For B-to-A Direction:
Prepare HBSS buffer with 0.4% DMSO and 5 μM LY: add 200 μL DMSO and 50 μL LY (5 mM) into 50 ml HBSS buffer (pH 7.4).

TABLE 4
Preparation of Test Solutions

| | | Stock Solution (in DMSO) | | | basolateral | Final DMSO |
|---|---|---|---|---|---|---|
| Compound | | Test cpd | Verapamil | apical Buffer | buffer | concentration |
| Erythromycin + | A-to-B dosing | 10 mM 3 μL | — | 0.1% DMSO | — | 0.4% |
| Metoprolol + | solution | | | HBSS + LY 3 mL | | |
| Atenolol | A-to-B Receiver | — | — | — | 0.4% DMSO | 0.4% |
| | solution | | | | HBSS | |
| | B-to-A dosing | 10 mM 3 μL | — | — | 0.1% DMSO | 0.4% |
| | solution | | | | HBSS 3 mL | |
| | B-to-A Receiver | — | — | 0.4% DMSO | — | 0.4% |
| | solution | | | HBSS + LY | | |
| cpds | A-to-B dosing | 10 mM 3 μL | — | 0.3% DMSO | — | 0.4% |
| | solution | | | HBSS + LY 3 mL | | |
| | A-to-B Receiver | — | — | — | 0.4% DMSO | 0.4% |
| | solution | | | | HBSS | |
| | B-to-A dosing | 10 mM 3 μL | — | — | 0.3% DMSO | 0.4% |
| | solution | | | | HBSS 3 mL | |
| | B-to-A Receiver | — | — | 0.4% DMSO | — | 0.4% |
| | solution | | | HBSS + LY | | |

4. Measure TEER Take cell culture plate out of incubator, wash the cell monolayers with HBSS buffer, and then measure TEER values at Rm temperature.
5. Centrifuge Centrifuge the compound solution (from step 3) at 4000 rpm for 5 min before loading to donor chambers.
6. Dosing Add solution based on the volumes listed in the following table (make sure to take extra 100 μL of donor sample for T₀ as Backup).

TABLE 5
Dosing Parameters

| Position | Transport Direction | Volume added | Final volume |
|---|---|---|---|
| Apical | A--B (Donor chamber) | 600 μL of A-to-B dosing solution (100 μL for | 400 μL |
| | | LY measurement and 100 μL for Backup) | |
| Basolateral | A--B (Receiver chamber) | 800 μL 0.4% DMSO HBSS | 800 μL |
| Basolateral | B--A (Donor chamber) | 900 μL B-to-A dosing solution (100 μL for | 800 μL |
| | | Backup) | |
| Apical | B--A (Receiver chamber) | 500 μL 0.4% DMSO HBSS+ LY (100 μL for | 400 μL |
| | | LY measurement) | |

7. Apical LYTO samples To determine LY concentration in the apical chamber, take 100 μL sample from apical chambers into an opaque plate for LYTO.
8. Pre-warm Pre-warm apical and basolateral plates at 37° C. for about 5 min, then begin transport by placing the apical plate onto basolateral plate.
9. Incubation Keep the plates in incubator at 37° C. for 90 min.
10. Standard Curve preparation Prepare 20× solution: For 300 μM compound solution, add 6 μL of compound stock solution into 192 μL of MeOH/H₂O (1:1).

### Prepare Working Solution in MeOH/H₂O (1:1)

TABLE 6
Solutions for Standard Curve Preparation

| Compound | | MeOH/H₂O | | Final |
|---|---|---|---|---|
| solution (μM) | Solution (μL) | (μL) | | solution (μM) |
| 300 | 100 | 400 | → | 60 |
| 60 | 100 | 200 | → | 20 |
| 20 | 100 | 400 | → | 4 |
| 4 | 100 | 400 | → | 0.8 |
| 0.8 | 100 | 300 | → | 0.2 |
| 0.2 | 100 | 100 | → | 0.1 |

Prepare 1× solution:
3 μL (20×)+57 μL of 0.4% DMSO HBSS+60 μL ACN with IS (Osalmid or Imipramine)—120 μL (1×)
11. Transport termination Separate the apical plate from the basolateral plate after 90-min incubation.
12. Measure LY Take 100 μL samples from basolateral plate to an opaque plate as LYT90.
13. Measure LY concentrations for LYTO and LYT90 by Fluorometer (at excitation of 485 nm/emission of 535 nm).
14. Sample preparation for LC-MS/MS Donor samples (1:10 diluted): 6 μL of donor sample+54 μL 0.4% DMSO HBSS+60 μL ACN with IS (Osalmid or Imipramine)
Receiver sample: 60 μL of receiver sample+60 μL ACN with IS (Osalmid or Imipramine)

TABLE 7
Bioanalytical Conditions

| Detection | LC-MS/MS-014(API4000) | | | | |
|---|---|---|---|---|---|
| method | | | | | |
| Matrix | HBSS | | | | |
| Internal | Osalmid or Imipramine | | | | |
| standard (s) | | | | | |
| MS conditions | Positive ion, ESI | | | | |
| Mobile phase | A: H₂O - 0.025% FA-1 mM NH₄OAC | | | | |
| | B: MeOH - 0.025% FA-1 mM NH₄OAC | | | | |
| Column | Ultimate-XB-C18 (2.1 × 50 mm, 5 μm) | | | | |
| | 0.60 mL/min | | | | |
| LC conditions | Time (min) | Pump B (%) | | | |
| | 0.2 | 2 | | | |
| | 0.4 | 98 | | | |
| | 1.40 | 98 | | | |
| | 1.41 | 2 | | | |
| | 2.50 | stop | | | |
| Detection & | | | | | |
| Retention time | | Analyte Mass | Analyte | IS Mass Ranges | IS RT |
| (RT) | Compound | Ranges (Da) | RT (min) | (Da) | (min) |
| | Erythromycin | 734.300/158.000 Da | 0.90 | 281.100/193.100 Da | 0.91 |
| | Metoprolol | 268.100/133.100 Da | 0.85 | 281.100/193.100 Da | 0.91 |
| | Atenolol | 267.000/145.100 Da | 0.78 | 281.100/193.100 Da | 0.91 |

### Results

Study details: Test concentration 10 μM
Reference compounds: Erythromycin, Metoprolol, Atenolol, Lucifer Yellow
Test systems: Caco-2/HBSS solution
Incubation conditions: 0, 90 min at 37° C.
Sample size: Duplicates (n=2)
Bioanalytical method: LC-MS/MS

### Calculations

Transepithelial electrical resistance (TEER)=(Resistance sample−Resistance blank)×Effective Membrane Area

Lucifer Yellow permeability: Papp=(VA/(Area×time))×([RFU]accepter−[RFU]blank)/(([RFU]initial, donor−[RFU]blank)×Dilution Factor)×100

Drug permeability: Papp=(VA/(Area×time))×([drug]accepter/(([drug]initial, donor)×Dilution Factor)
Where VA is the volume in the acceptor well, area is the surface area of the membrane and time is the total transport time in seconds.

For Millicell-24 Cell Culture Plates: surface area of the membrane=0.7 cm², VA=0.8 mL (A-to-B) or 0.4 mL (B-to-A)

### Results

TEER value of Caco-2 monolayers from randomly selected wells was 357±29 Ω·cm² (Mean±SD). Note: Cell monolayer is used if TEER value >100 Ω·cm².

Comments:
1. Papp values were calculated based on calculated concentrations.
2. Most of the Caco-2 monolayers applied in this assay showed intact tight junctions as indicated by TEER values and low permeability for Lucifer Yellow, a low permeability control (data not shown).
3. Metoprolol, a high permeability control, showed both A-to-B and B-to-A permeability >10×10-6 cm/sec in Caco-2 cells. Atenolol, a low permeable control, showed both A-to-B and B-to-A permeability less than 5×10−6 cm/sec in Caco-2 cells. Erythromycin, an efflux substrate, gave an efflux ratio higher than 116.11 in Caco-2 cells.
4. As summarized in Table 8, compounds showing permeability <5×10−6 cm/sec suggest low permeability; compounds showing permeability 5 to OX10-6 cm/sec suggest moderate permeability in A-to-B direction; compounds showing permeability >OX10-6 cm/sec suggest high permeability.

Permeability results are shown in Table 8 for selected compounds of the invention. The compound numbers correspond to the compound numbers in Table 1. Compounds having a ratio designated as “A” provided a ratio of 0.1 to 10; compounds having a ratio designated as “B” provided a ratio of >10 to <30; and compounds having a ratio designated as “C” provided a ratio of 30 or greater.

Compounds not assayed are denoted “N/A.”

TABLE 8
Caco-Papp Permeability for Selected Compounds

| Compound | Caco-Papp (A-B) | Caco-Papp (B-A) | Efflux ratio |
|---|---|---|---|
| # | (10⁻⁶ cm/sec) | (10⁻⁶ cm/sec) | (PB-A/PA-B) |
| I-1 | N/A | N/A | N/A |
| I-2 | High | High | A |
| I-3 | High | High | A |
| I-4 | High | High | A |
| I-5 | High | High | A |
| I-6 | Low | High | B |
| I-7 | Low | High | C |
| I-8 | Low | High | B |
| I-9 | Low | High | B |
| I-10 | Moderate | High | A |
| I-11 | Moderate | High | A |
| I-12 | Low | High | C |
| I-13 | High | High | A |
| I-14 | High | High | A |
| I-15 | High | High | A |
| I-16 | High | High | A |
| I-17 | N/A | N/A | N/A |
| I-18 | N/A | N/A | N/A |
| I-19 | High | High | A |
| I-20 | High | High | A |
| I-21 | Low | High | C |
| I-22 | Moderate | High | B |
| I-23 | Moderate | High | B |
| I-24 | High | High | A |
| I-25 | Low | High | B |
| I-26 | Low | High | B |
| I-27 | Moderate | High | A |
| I-28 | High | High | A |
| I-29 | N/A | N/A | N/A |
| I-30 | N/A | N/A | N/A |
| I-31 | High | High | A |
| I-32 | High | High | A |
| I-33 | High | High | A |
| I-34 | Moderate | High | A |
| I-35 | High | High | A |
| I-36 | Low | High | B |
| I-37 | Moderate | High | A |
| I-38 | Moderate | High | A |
| I-39 | High | High | A |
| I-40 | Low | High | B |
| I-41 | Low | High | C |
| I-42 | Low | High | C |
| I-43 | Moderate | High | A |
| I-44 | Moderate | High | A |
| I-45 | Low | High | A |
| I-46 | High | High | A |
| I-47 | High | High | A |
| I-48 | Low | High | A |
| I-49 | Moderate | High | A |
| I-50 | High | High | A |
| I-51 | Moderate | High | A |
| I-52 | Moderate | High | A |
| I-53 | High | High | A |
| I-54 | Moderate | High | A |
| I-55 | High | High | A |
| I-56 | Moderate | High | A |
| I-57 | Low | High | A |
| I-58 | High | High | A |
| I-59 | High | High | A |

### Example 13: Pharmacokinetics and Brain Penetration Experiment to Determine Brain and Plasma Concentrations of Compounds after IV Administration to Male CD1 Mice or Male SD Rat

### Mouse Study

In-life summary: The study design consist of administrating the drug (IV: 3 mg/kg (5 mL/kg) via tail vein injection) and collecting samples at terminal bleeding for plasma and brain at 0.083, 0.5 and 1 h. The blood collection are to be performed as follows: restrain the animal manually and collect approximately 150 μL blood/time point into a dipotassium EDTA tube via retro orbital puncture under anesthesia with isoflurane. Put the blood sample on ice and centrifuge to obtain a plasma sample (2000 g, 5 min under 4° C.) within 15 minutes. The brain collection are to be performed as follows: make a mid-line incision in the animal's scalp and retract the skin. Using small bone cutters and rongeurs, remove the skull overlying the brain. Remove the brain using a spatula and rinse the brain with cold saline. Place the brain in screw-top tubes, and store the tubes at −70° C. until analysis. Prepare the IV dosing solution in 50 mM citrate buffer (pH 4.0) at 0.6 mg/mL.

Plasma sample preparation: Add an aliquot of 30 μL sample to 150 μL MeCN containing 50 ng/mL IS (Dexamethasone). Vortex the mixture for 5 min and centrifuge at 14,000 rpm for 5 min. Inject an aliquot of 5 μL supernatant for LC-MS/MS analysis.

Brain sample preparation: Add an aliquot of 30 μL brain homogenate (brain:PBS=1:3, w/v) sample to 150 μL IS in ACN (Dexamethasone, 50 ng/mL). Vortex the mixture for 5 min and centrifuge at 14,000 rpm for 5 min. Inject an aliquot of 5 μL supernatant for LC-MS/MS analysis.

Analytical Method: The sample analysis are to be performed on LCMS/MS-003 (API4000, triple quadruple) under the following conditions: positive ion, ESI, MRM detection using dexamethasone as internal standard. HPLC conditions: mobile phase A: H₂O (0.025% formic acid (FA) with 1 mM NH₄OAc); mobile phase B: MeOH (0.025% FA with 1 mM NH₄OAc) on Waters X-Bridge C18 (2.1×50 mm, 2.5 μm) column at 60° C.

### Rat Study

In-life summary: The study design consists of 2 groups and administrating the drug [IV: 3 mg/kg (1.5 mL/kg) via foot dorsal vein], [PO: 10 mg/kg (5 mL/kg) via oral gavage] and collecting samples at terminal bleeding for plasma, brain and CSF at 0.25, 0.5, 1, 4, 8 and 24 hr. Prepare the IV and PO dosing solutions in 50 mM citrate buffer (pH 4.0) at 2 mg/mL. Perform the blood collection as follows: restrain the animal manually at the designated time points, collect approximately 150 μL of blood sample via cardiac puncture vein into EDTA-2K tubes. Maintain the blood samples in wet ice first and centrifuge to obtain plasma (2000 g, 4° C., 5 min) within 15 minutes post sampling. The brain collection are to be performed as follows: make a mid-line incision in the animal's scalp and retract the skin. Using small bone cutters and rongeurs, remove the skull overlying the brain. Remove the brain using a spatula and rinse with cold saline. Place the brain in screw-top tubes and then store under −70° C. until analysis. The CSF collection will be performed as follows: euthanize the animal under deep anesthesia with air bubble tail vein injection. Collect the CSF by direct puncture of butterfly needle into the cisterna magna, using the occipital bone and the wings of the atlas as landmarks. Use a piece of white paper as a background to monitor color change in the sample just above the needle during collection. Upon observation of color change, quickly clamp the PE tubing off above the color change and cut just above the clamped site. Draw the clear sample into the syringe.

Plasma samples preparation: Add an aliquot of 30 μL sample to 100 μL MeCN containing 100 ng/mL IS (Dexamethasone). Vortex the mixture for 10 min and centrifuge at 5800 rpm for 10 min. Add an aliquot of 40 μL supernatant to 40 μl H₂O and vortex the mixture for 5 min. Inject an aliquot of 2 μL supernatant for LC-MS/MS analysis.

Brain samples preparation: Homogenize the sample with 3 volumes (v/w) of PBS. Add an aliquot of 30 μL sample to 100 μL ACN containing 100 ng/mL IS (Dexamethasone). Vortex the mixture for 10 min and centrifuge at 5800 rpm for 10 min. Add an aliquot of 40 μL supernatant to 40 μl H₂O and vortex the mixture for 5 min. inject an aliquot of 2 μL supernatant for LC-MS/MS analysis.

CSF samples preparation: Add an aliquot of 10 μL sample to 10 μL MeOH/H₂O (1/1) and 40 μL ACN containing 200 ng/mL IS (Dexamethasone) 120 μL H₂O. Vortex the mixture for 5 min. Inject an aliquot of 2 μL supernatant for LC-MS/MS analysis.

Analytical Method: The sample analysis will be performed on UPLC-MS/MS-02 (Triple Quad™ 4000) under the following conditions: positive ion, ESI, MRM detection using dexamethasone as internal standard. HPLC conditions: mobile phase A: H₂O-0.1% FA, mobile phase B: MeCN-0.1% FA on ACQUITY UPLC HSS T3 (2.1×50 mm, 1.8 μm) column at 60° C.

### Example 14: MTD and Pharmacokinetics and Brain Penetration Experiment to Determine Brain and Plasma Concentration of Compounds after PO Administration to Male C57BL/6 Mice

In-life summary: The study will be designed with 2 groups consists of administrating the drug [PO-50, 100, 150, 225, 300 mg/kg via oral gavage] and collecting samples at terminal bleeding for plasma, brain and CSF at 0.25, 0.5, 1, 4, 8, and 24 hr. All PO dosing solutions are to be prepared in 50 mM citrate buffer (pH 4.0).

TABLE 11
Administration of Compounds Schedule for Two Test Groups

| | |
|---|---|
| Group 1: Single | PO: 50 mg/kg (10 mL/kg) via oral gavage |
| administration: | PO-day 1: 50 mg/kg (10 mL/kg) via oral gavage |
| Group 2: Multiple | PO-day2: 100 mg/kg (10 mL/kg) via oral gavage |
| administrations: | PO-day3: 150 mg/kg (10 mL/kg) via oral gavage |
| | PO-day4: 225 mg/kg (10 mL/kg) via oral gavage |
| | PO-day5: 300 mg/kg (10 mL/kg) via oral gavage |

The blood collection will be performed as follows: restrain the animal manually at the designated time points, collect approximately 500 μL of blood sample via cardiac puncture vein into EDTA-2K tubes. Divide the whole blood needed into two parts; place one part in the tube containing EDTA-2K for plasma generation and use the other part for the hematology assay. Maintain the blood samples for plasma generation in wet ice first and centrifuge to obtain plasma (2000 g, 4° C., 5 min) within 15 minutes post sampling. The brain collection will be performed as follows: make a mid-line incision in the animal's scalp and retract the skin. Using small bone cutters and rongeurs, remove the skull overlying the brain. Remove the brain using a spatula and rinse with cold saline. Place the brain in screw-top tubes, and store at −70° C. until analysis. The CSF collection will be performed as follows: make a mid-line incision on the neck. Cut the muscle under the skin to expose the cisterna magna. Penetrate the cisterna magna with the sharp end of one capillary (Burn one end of capillary to make it sharp). Suck the CSF into the capillary (will occur spontaneously).

Plasma sample preparation: Add an aliquot of 30 μL sample to 100 μL MeCN containing 100 ng/mL IS (Dexamethasone). Vortex the mixture for 10 min and centrifuge at 5800 rpm for 10 min. Add an aliquot of 40 μL supernatant to 40 μL H₂O and vortex the mixture for 5 min. Inject an aliquot of 2 μL supernatant for LC-MS/MS analysis.

Brain sample preparation: Add an aliquot of 30 μL brain homogenate (brain:PBS=1:3, w/v) sample to 100 μL MeCN containing 100 ng/mL IS (Dexamethasone). Vortex the mixture for 10 min and centrifuge at 5800 rpm for 10 min. Add an aliquot of 40 μL supernatant to 40 μL H₂O and vortex the mixture for 5 min. Inject an aliquot of 2 μL supernatant for LC-MS/MS analysis.

CSF samples preparation: Add an aliquot of 3 μL sample to a mixture of 6 μL CSF, 9 μL MeOH/H₂O (1/1), 40 μL MeCN containing 200 ng/mL IS (Dexamethasone), and 116 μL H₂O. Vortex the mixture for 5 min. Inject an aliquot of 4 μL for LC-MS/MS analysis.

Analytical Method: The sample analysis will be performed on UPLC-MS/MS-02 (Triple Quad™ 4000) under the following conditions: positive ion, ESI, MRM detection using dexamethasone as internal standard. HPLC conditions: mobile phase A: H₂O-0.1% formic acid, mobile phase B: MeCN-0.1% formic acid on: ACQUITY UPLC HSS T3 (2.1×50 mm, 1.8 μm) at 60° C.

### Example 15: 7-Day Toxicology Study in Mice

### Toxicology Summary

A toxicology study may be performed as described in this Example. Overt signs of toxicity after 7 days of repeat dosing up to 100 mg/kg P.O. in terms of clinical observations, body weight or food consumption will be examined. White blood cells will be monitored and internal organs examined after necroscopy.

TABLE 14
Toxicology Study Design

| | |
|---|---|
| Test system | C57BL/6 Mouse, 5 weeks old, 18-20 g, male, N = 12 |
| Food status | Free access to food and water |
| Administration | Group 1: 0 mg/kg/day (10 mL/kg/day) via oral gavage |
| | (N = 3) |
| | Group 2: 10 mg/kg/day (10 mL/kg/day) via oral gavage |
| | (N = 3) |
| | Group 3: 30 mg/kg/day (10 mL/kg/day) via oral gavage |
| | (N = 3) |
| | Group 4: 100 mg/kg/day (10 mL/kg/day) via oral gavage |
| | (N = 3) |

### Toxicokinetics

Mean plasma, brain and CSF concentration-time profiles of test compounds will be measured after a single PO administration at 30 mg/kg in male C57BL/6 mice (5 weeks old) (N=3/time point). Mean plasma, brain and CSF concentration-time profiles of compounds after repeat PO administrations at 30 mg/kg in male C57BL/6 mice on day 7 (5 weeks old) (N=3/time point) will also be measured.

In-life summary: The study design (36 animals, C57BL/6 mouse) consists of administrating the drug [PO: 30 mg/kg/day (10 mL/kg/day) via oral gavage] and collecting samples at terminal bleeding for plasma, brain and CSF at 0.025, 0.5, 1, 4, 8 and 24 hr. The PO dosing solutions will be prepared in 50 mM citrate buffer (pH 4.0) at 3 mg/mL. The blood collection will be performed as follows: the animal will be anesthetized under isoflurane. Approximately 500 μL blood/time point will be collected into K₂EDTA tube via cardiac puncture for terminal bleeding. Approximately 200 μL blood samples will be put on ice and centrifuged to obtain a plasma sample (2000 g, 5 min under 4° C.) within 15 minutes of collection. Approximately 300 blood samples will be used for hematology assay. The brain collection will be performed as follows: a mid-line incision will be made in the animal's scalp and skin retracted. The skull overlying the brain will be removed. The whole brain will be collected, rinsed with cold saline, dried on filtrate paper, weighted, and snap frozen by placing into dry ice. The brain sample will be homogenized for 2 min with 3 volumes of PBS (pH 7.4) by Mini-bead-beater before sample extraction.

Plasma samples preparation: An aliquot of 10 μL sample will be added to 200 μL MeCN containing 10 ng/mL IS (Glipizide). The mixture will be vortexed for 10 min and centrifuged at 6,000 rpm for 10 min. An aliquot of 1 μL constitution will be injected for LC-MS/MS analysis.

CSF samples preparation: An aliquot of 3 μL sample will be added to 70 μL MeCN containing 10 ng/mL IS (Glipizide). The mixture will be vortexed for 2 min and centrifuged at 14,000 rpm for 5 min. An aliquot of 1 μL constitution will be injected for LC-MS/MS analysis.

Tissue samples preparation: The sample will be homogenized with 3 volumes (v/w) of PBS. An aliquot of 10 μL sample will be added to 200 μL MeCN containing 10 ng/mL IS (Glipizide). The mixture will be vortexed for 10 min and centrifuged at 6,000 rpm for 10 min. An aliquot of 1 μL constitution will be injected for LC-MS/MS analysis.

Analytical Method: The sample analysis will be performed on LCMSMS-2 (Triple Quad 6500+) under the following conditions: positive ion, ESI, MRM detection using glipizide as internal standard. HPLC conditions: mobile phase A: H₂O/0.025% FA with 1 mM NH₄OAc, mobile phase B: MeOH/0.025% FA with 1 mM NH₄OAc on Waters X-Bridge BEH C18 (2.1×50 mm, 2.5 μm) column at 60° C.

### Example 16: Pharmacokinetics of Compounds after Intravenous or Oral Administration to Male Beagle Dogs

In-life summary: The study design (9 animals, fasted overnight and fed at 4 h post dosing) consists of administrating the drug [IV: 1 mg/kg via cephalic vein injection], [PO: 3 mg/kg and 10 mg/kg via oral gavage] and collecting samples at serial bleeding for plasma at 0.03, 0.08, 0.25, 0.5, 1, 2, 4, 8, 24, 48 and 72 hr. Prepare the IV and PO dosing solutions in 50 mM citrate buffer (pH 4.0) at 0.5 mg/mL, 1.5 mg/mL and 5 mg/mL, respectively. The blood collection will be performed as follows: restrain the animals manually, and collect approximately 0.5 mL blood/time point from the cephalic vein into pre-cooled K₂EDTA tubes. Put blood samples on wet ice and centrifuge at 4° C. to obtain plasma within 15 minutes of sample collection. Store all samples at approximately −70° C. until analysis.

Plasma samples preparation: Add an aliquot of 30 μL sample to 100 μL MeCN containing 200 ng/mL IS (Dexamethasone). Vortex the mixture for 10 min and centrifuge at 5,800 rpm for 10 min. Add an aliquot of 30 μL supernatant to 60 μL H₂O and vortex the mixture for 5 min. Inject an aliquot of 4 μL supernatant for LC-MS/MS analysis.

Analytical Method: The sample analysis will be performed using UPLC-MS/MS-02 (Triple Quad™ 4000) under the following conditions: positive ion, ESI, MRM detection using dexamethasone as internal standard. HPLC conditions: mobile phase A: H₂O-0.1% FA, mobile phase B: ACN-0.1% FA on ACQUITY UPLC HSS T3 (2.1×50 mm, 1.8 μm) column at 60° C.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of Formula I: 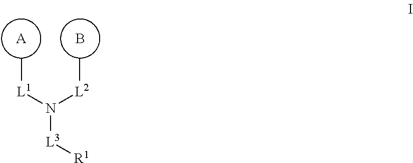
or a pharmaceutically acceptable salt thereof, wherein:
Ring A is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
Ring B is an optionally substituted ring selected from a 5-6 membered monocyclic heteroaromatic ring having 1-4 heteroatoms independently selected from nitrogen, oxygen, or sulfur, or an 8-10 membered bicyclic partially unsaturated or aromatic ring having 0-3 heteroatoms independently selected from nitrogen, oxygen, or sulfur;
L¹ is —CH₂— or —CH(CH₃)—;
L² is a covalent bond, —CH₂—, or —CH(CH₃)—;
L³ is a C₂₋₃ bivalent straight or branched hydrocarbon chain;
R¹ is -Cy, —OR, —N(R)₂, —C(O)N(R)₂, or —N(R)C(O)R;
each R is independently hydrogen or an optionally substituted group selected from C₁₋₆ aliphatic, a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur,
or two R groups on the same nitrogen are optionally take together with their intervening atoms to form a 5-6 membered saturated, partially unsaturated, or aromatic heterocyclic ring having 1-2 heteroatoms in addition to the nitrogen attached thereto independently selected from nitrogen, oxygen, or sulfur; and
-Cy is an optionally substituted ring selected from a 3-8 membered saturated or partially unsaturated monocyclic carbocyclic ring, phenyl, an 8-10 membered bicyclic aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur;
wherein the compound is other than a compound selected from.

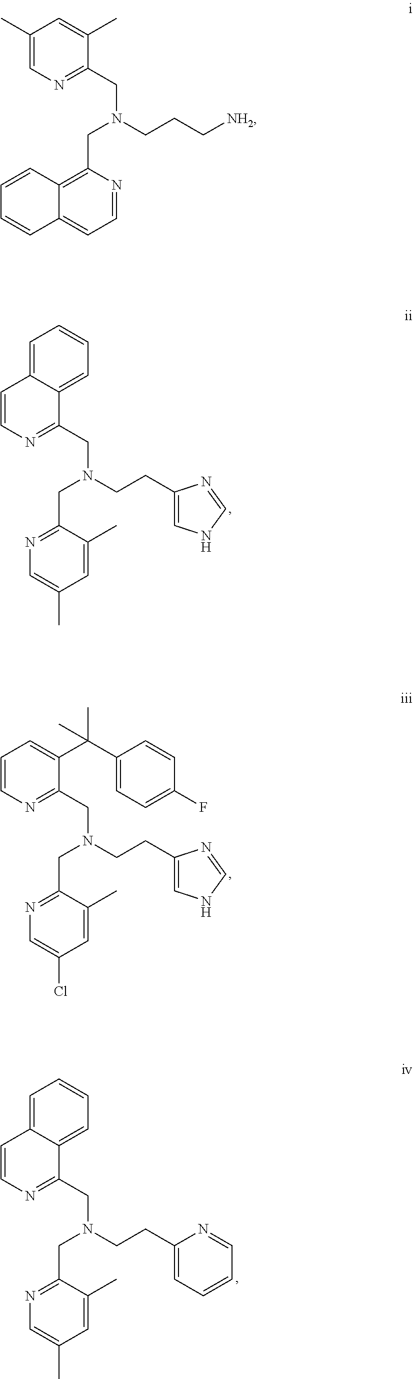 [SMILES: Cc1cnc(CN(CCCN)Cc2nccc3ccccc23)c(C)c1 ; Cc1cnc(CN(CCc2c[nH]cn2)Cc2nccc3ccccc23)c(C)c1 ; Cc1cc(Cl)cnc1CN(CCc1c[nH]cn1)Cc1ncccc1C(C)(C)c1ccc(F)cc1 ; Cc1cnc(CN(CCc2ccccn2)Cc2nccc3ccccc23)c(C)c1]

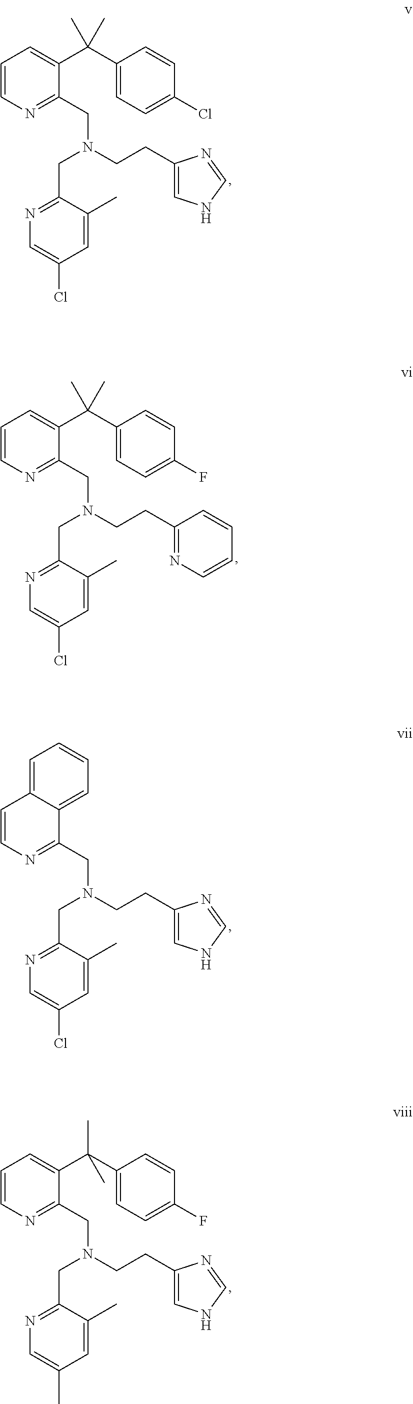 [SMILES: Cc1cc(Cl)cnc1CN(CCc1c[nH]cn1)Cc1ncccc1C(C)(C)c1ccc(Cl)cc1 ; Cc1cc(Cl)cnc1CN(CCc1ccccn1)Cc1ncccc1C(C)(C)c1ccc(F)cc1 ; Cc1cc(Cl)cnc1CN(CCc1c[nH]cn1)Cc1nccc2ccccc12 ; Cc1cnc(CN(CCc2c[nH]cn2)Cc2ncccc2C(C)(C)c2ccc(F)cc2)c(C)c1]

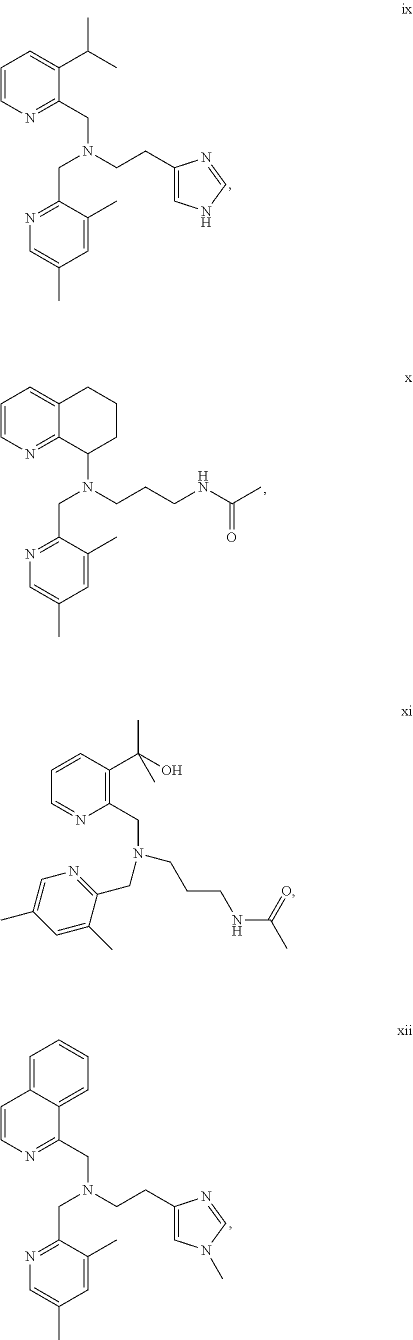

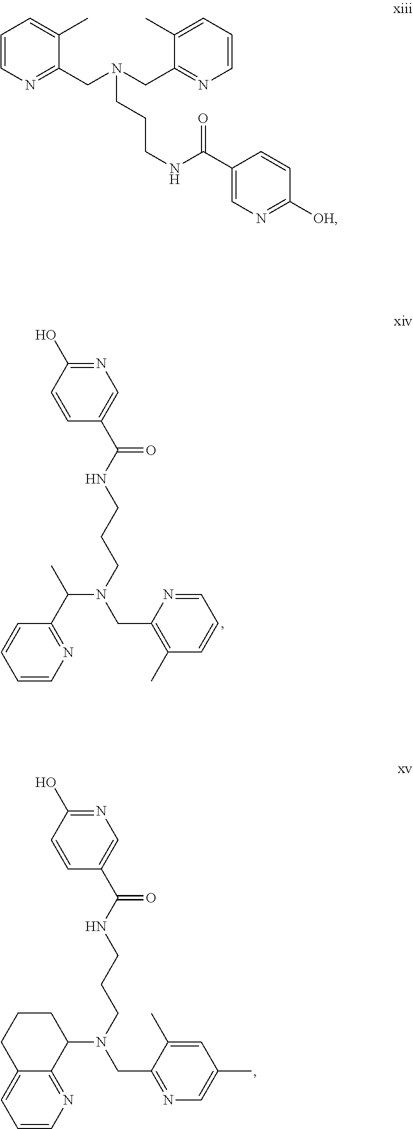 [SMILES: Cc1cccnc1CN(CCCNC(=O)c1ccc(O)nc1)Cc1ncccc1C ; Cc1cccnc1CN(CCCNC(=O)c1ccc(O)nc1)C(C)c1ccccn1 ; Cc1cnc(CN(CCCNC(=O)c2ccc(O)nc2)C2CCCc3cccnc32)c(C)c1]

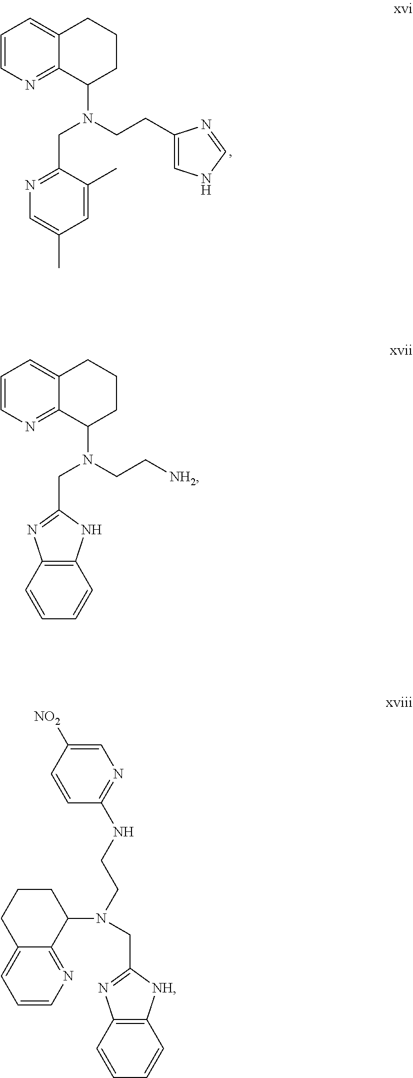 [SMILES: Cc1cnc(CN(CCc2c[nH]cn2)C2CCCc3cccnc32)c(C)c1 ; NCCN(Cc1nc2ccccc2[nH]1)C1CCCc2cccnc21 ; O=[N+]([O-])c1ccc(NCCN(Cc2nc3ccccc3[nH]2)C2CCCc3cccnc32)nc1]

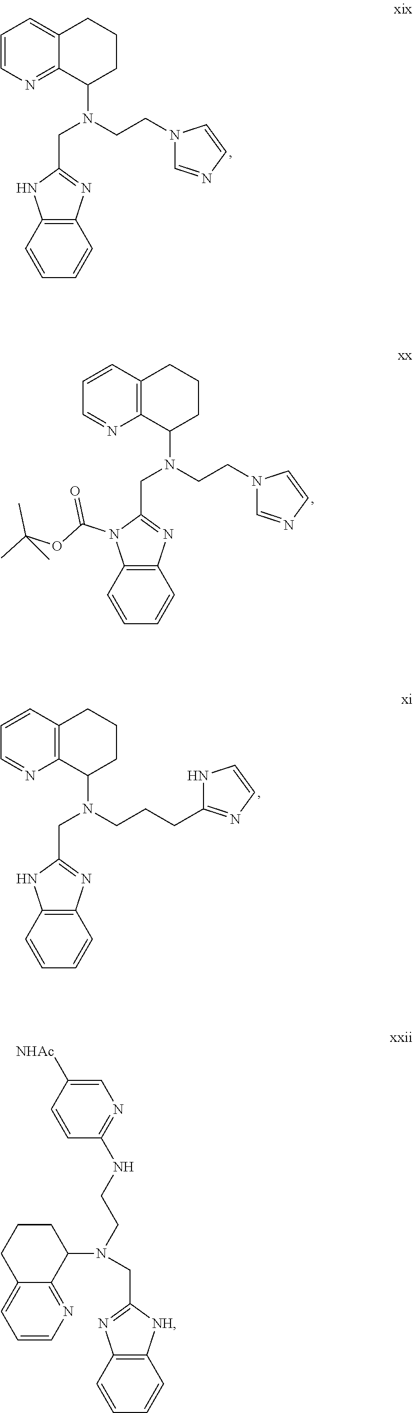 [SMILES: c1cnc2c(c1)CCCC2N(CCn1ccnc1)Cc1nc2ccccc2[nH]1 ; CC(C)(C)OC(=O)n1c(CN(CCn2ccnc2)C2CCCc3cccnc32)nc2ccccc21 ; c1cnc2c(c1)CCCC2N(CCCc1ncc[nH]1)Cc1nc2ccccc2[nH]1 ; CC(=O)Nc1ccc(NCCN(Cc2nc3ccccc3[nH]2)C2CCCc3cccnc32)nc1]

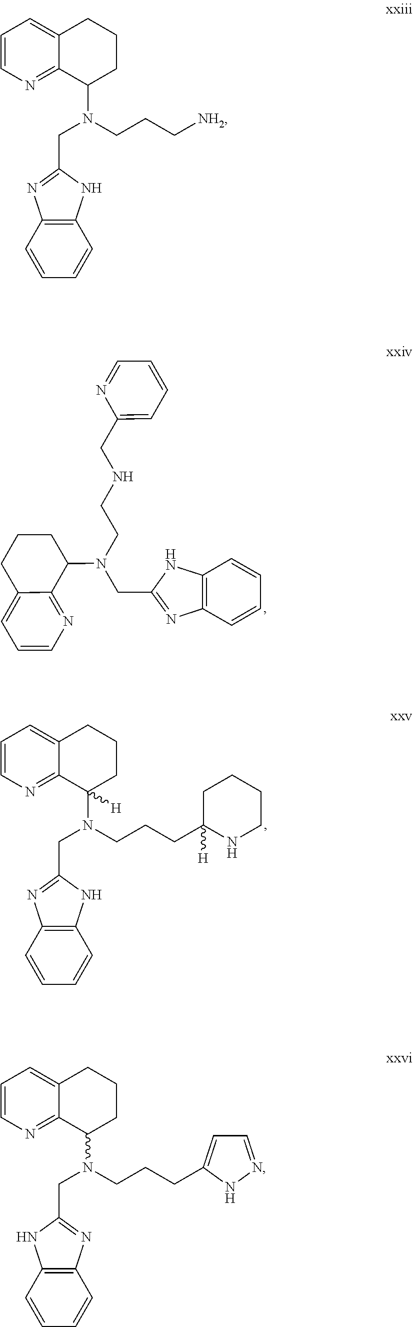 [SMILES: NCCCN(Cc1nc2ccccc2[nH]1)C1CCCc2cccnc21 ; c1ccc(CNCCN(Cc2nc3ccccc3[nH]2)C2CCCc3cccnc32)nc1 ; c1cnc2c(c1)CCCC2N(CCCC1CCCCN1)Cc1nc2ccccc2[nH]1 ; c1cnc2c(c1)CCCC2N(CCCc1ccn[nH]1)Cc1nc2ccccc2[nH]1]

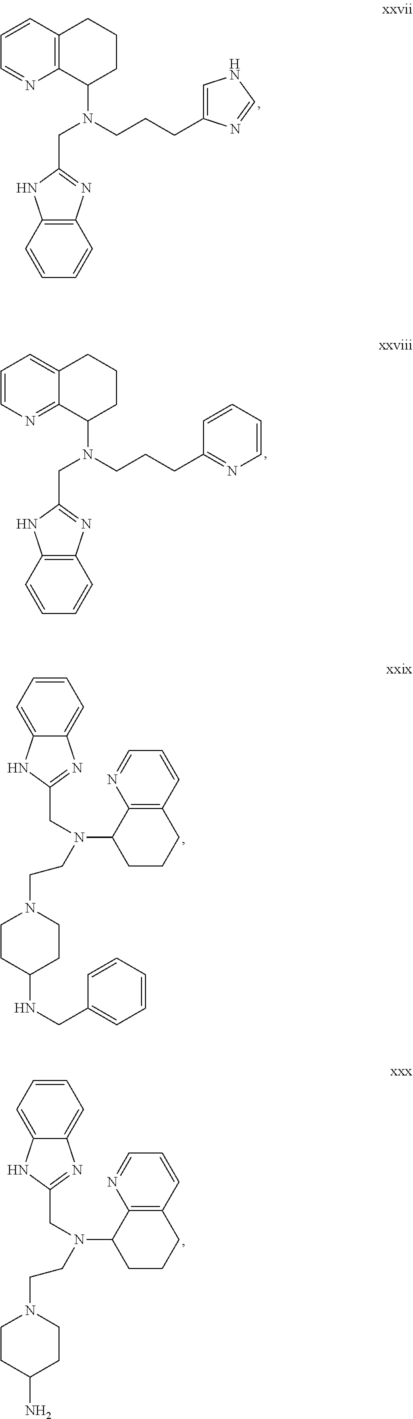 [SMILES: c1cnc2c(c1)CCCC2N(CCCc1c[nH]cn1)Cc1nc2ccccc2[nH]1 ; c1ccc(CCCN(Cc2nc3ccccc3[nH]2)C2CCCc3cccnc32)nc1 ; c1ccc(CNC2CCN(CCN(Cc3nc4ccccc4[nH]3)C3CCCc4cccnc43)CC2)cc1 ; NC1CCN(CCN(Cc2nc3ccccc3[nH]2)C2CCCc3cccnc32)CC1]

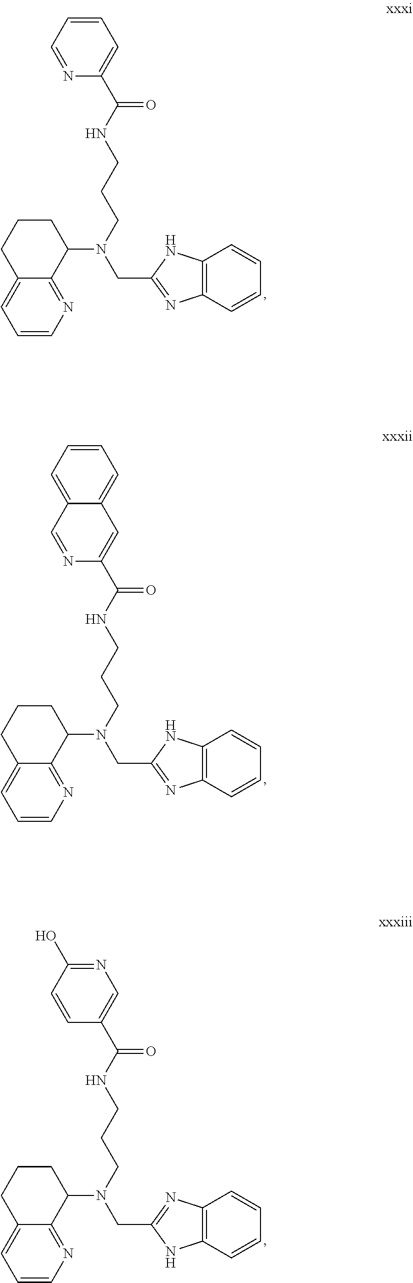 [SMILES: O=C(NCCCN(Cc1nc2ccccc2[nH]1)C1CCCc2cccnc21)c1ccccn1 ; O=C(NCCCN(Cc1nc2ccccc2[nH]1)C1CCCc2cccnc21)c1cc2ccccc2cn1 ; O=C(NCCCN(Cc1nc2ccccc2[nH]1)C1CCCc2cccnc21)c1ccc(O)nc1]

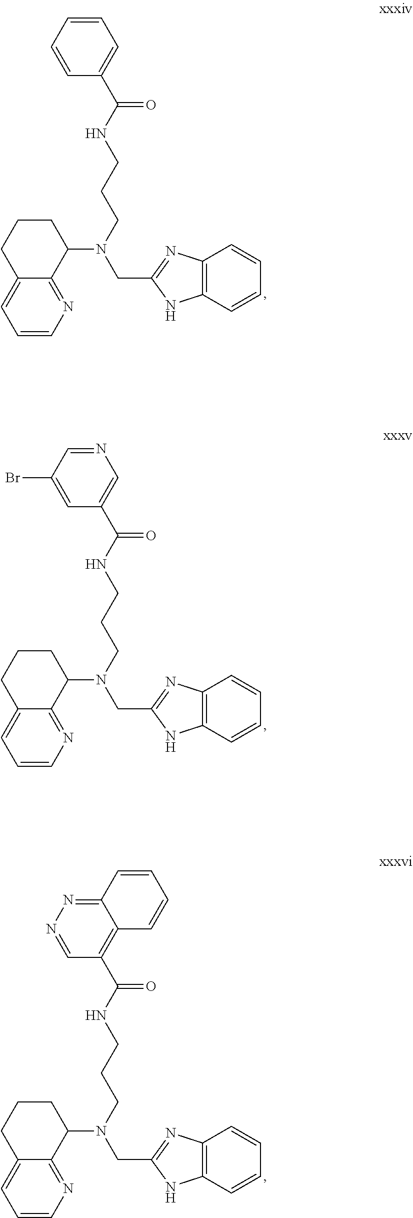 [SMILES: O=C(NCCCN(Cc1nc2ccccc2[nH]1)C1CCCc2cccnc21)c1ccccc1 ; O=C(NCCCN(Cc1nc2ccccc2[nH]1)C1CCCc2cccnc21)c1cncc(Br)c1 ; O=C(NCCCN(Cc1nc2ccccc2[nH]1)C1CCCc2cccnc21)c1cnnc2ccccc12]

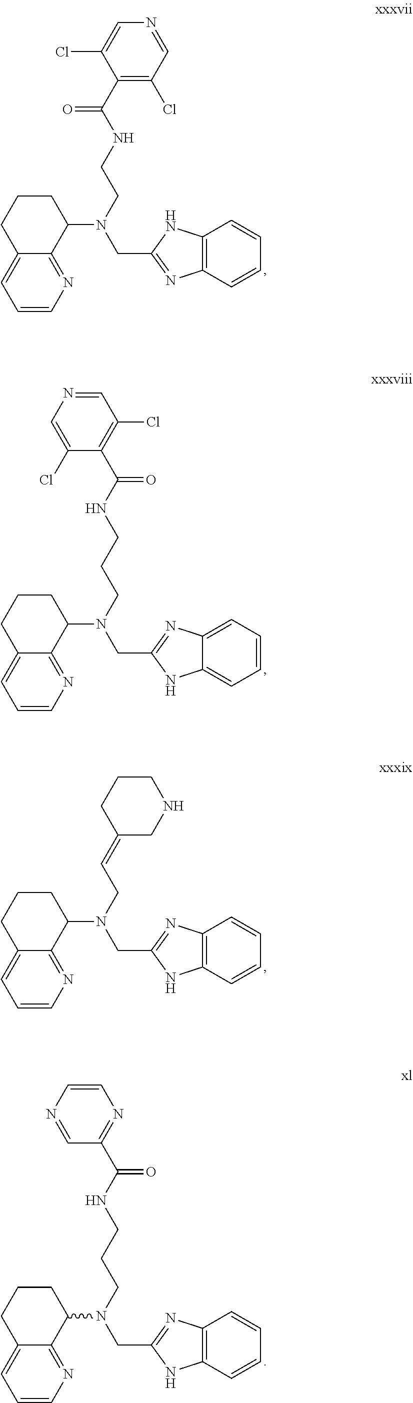 [SMILES: O=C(NCCN(Cc1nc2ccccc2[nH]1)C1CCCc2cccnc21)c1c(Cl)cncc1Cl ; O=C(NCCCN(Cc1nc2ccccc2[nH]1)C1CCCc2cccnc21)c1c(Cl)cncc1Cl ; C(/CN(Cc1nc2ccccc2[nH]1)C1CCCc2cccnc21)=C1\CCCNC1 ; O=C(NCCCN(Cc1nc2ccccc2[nH]1)C1CCCc2cccnc21)c1cnccn1]

2. The compound according to claim 1, wherein Ring A is selected from

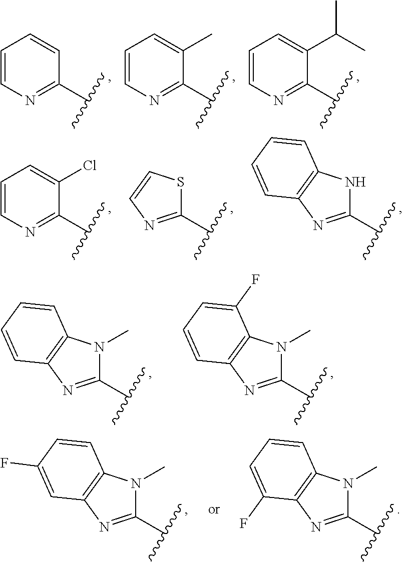

3. The compound according to claim 2, wherein Ring B is an optionally substituted ring selected from N

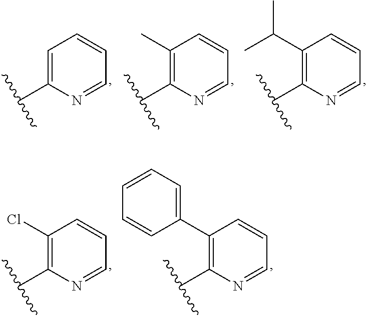

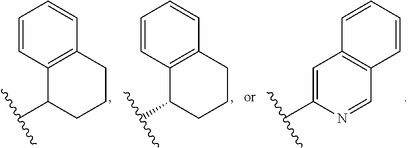

4. The compound according to claim 1, wherein R¹ is an optionally substituted ring selected from

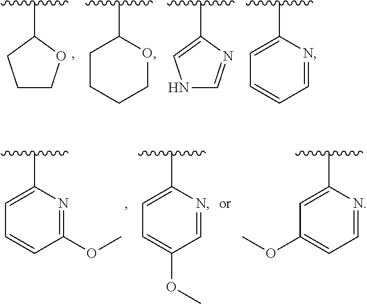

5. The compound according to claim 3, wherein L¹ is selected from —CH₂— or —CH(CH₃)—.

6. The compound according to claim 5, wherein L² is —CH₂—.

7-9. (canceled)

10. The compound according to claim 1, wherein the compound is of one of Formulae V-a or V-b: 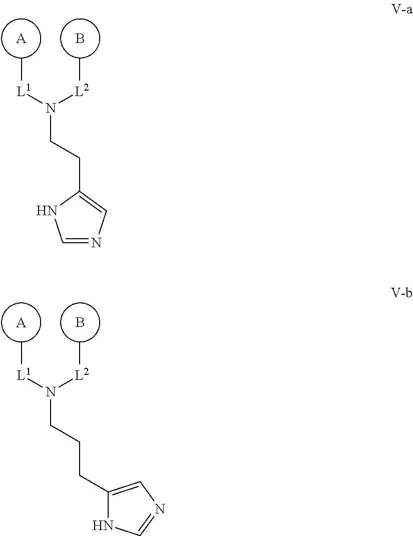
or a pharmaceutically acceptable salt thereof.

11. The compound according to claim 1, wherein the compound is of one of Formulae VIII-a, VIII-b, VIII-c, or VIII-d: 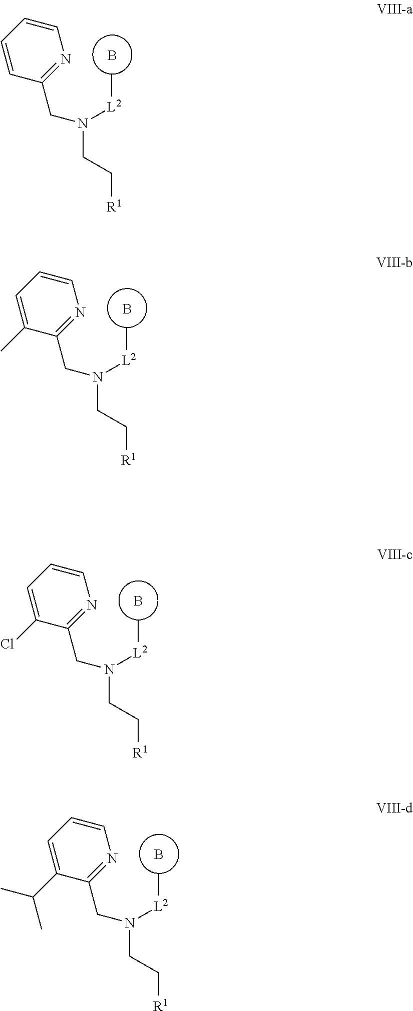
or a pharmaceutically acceptable salt thereof.

12. The compound according to claim 1, wherein the compound is of one of Formulae IX-a, IX-b, or IX-c: 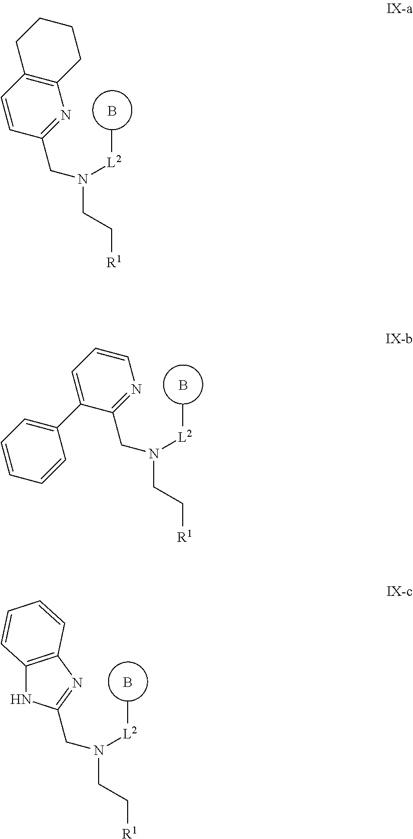
or a pharmaceutically acceptable salt thereof.

13. The compound according to claim 1, wherein the compound is of one of Formulae X-a, X-b, X-c, or X-d: 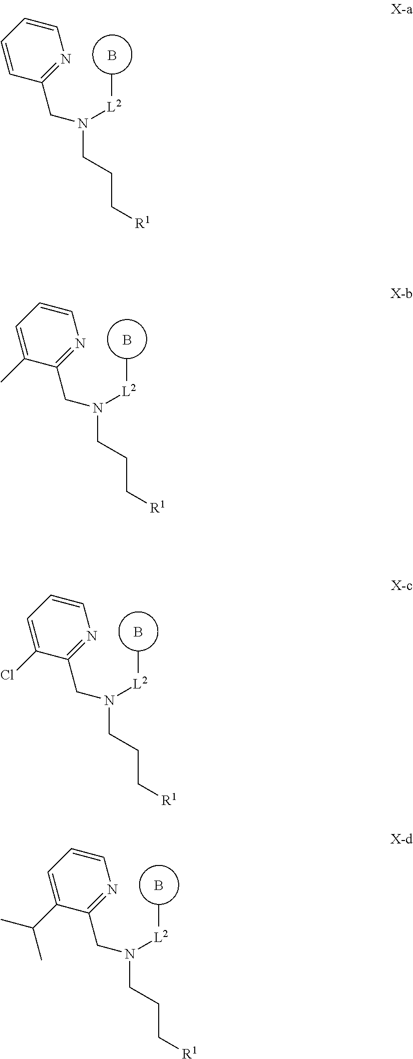
or a pharmaceutically acceptable salt thereof.

14. The compound according to claim 1, wherein the compound is of one of Formulae XI-a, XI-b, or XI-c: 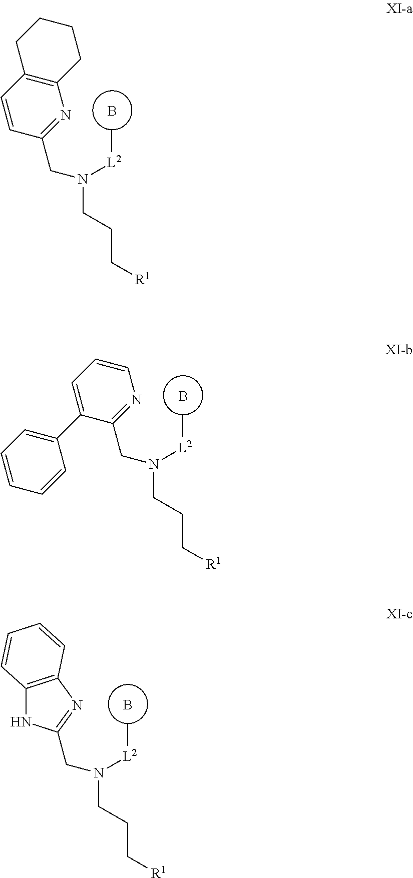
or a pharmaceutically acceptable salt thereof.

15. The compound according to claim 1, wherein the compound is of one of Formulae XII-a, XII-b, or XII-c: 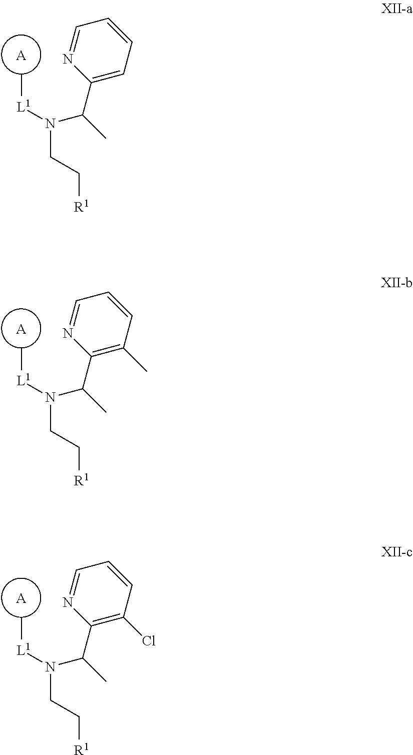
or a pharmaceutically acceptable salt thereof.

16. The compound according to claim 1, wherein the compound is of one of Formulae XIII-a, XIII-b, or XIII-c: 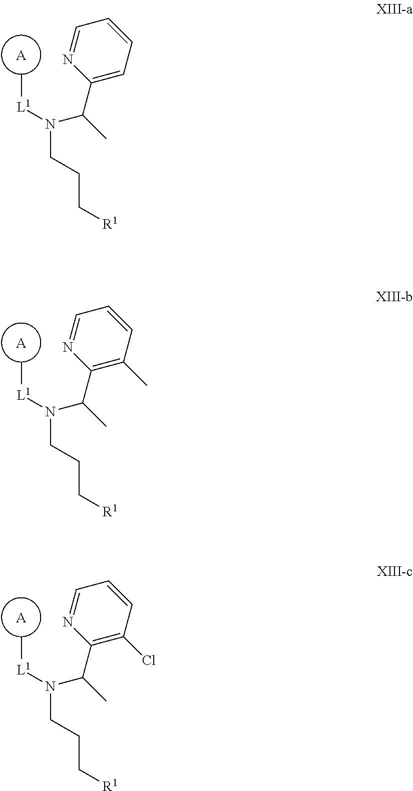
or a pharmaceutically acceptable salt thereof.

17. The compound according to claim 1, wherein the compound is of one of Formulae XIV-a, XIV-b, XIV-c, or XIV-d: 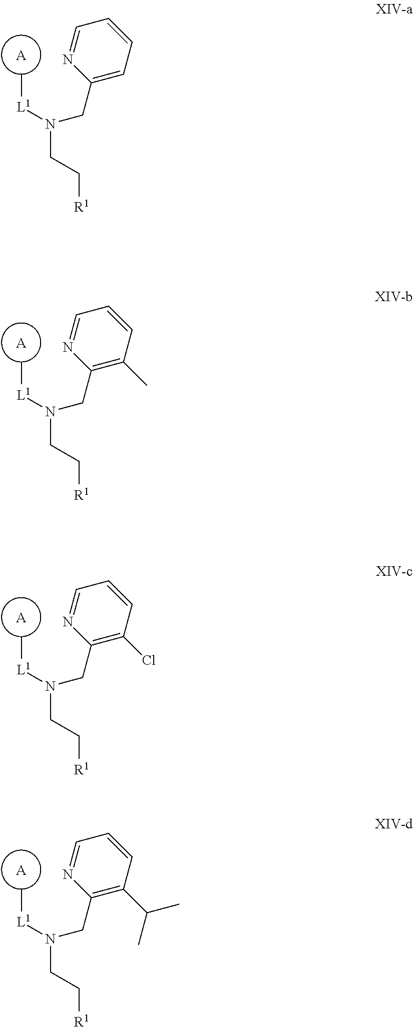
or a pharmaceutically acceptable salt thereof.

18. The compound according to claim 1, wherein the compound is of one of Formulae XV-a, XV-b, XV-c, or XV-d: 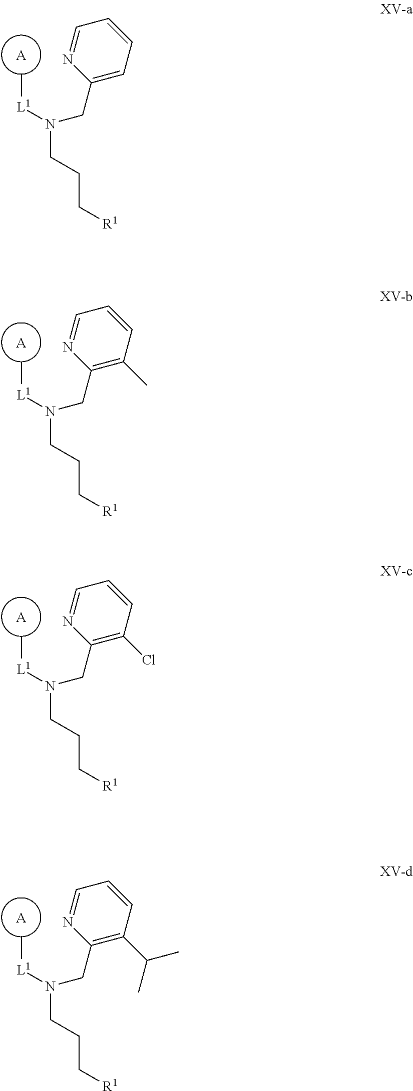
or a pharmaceutically acceptable salt thereof.

19. The compound according to claim 1, wherein the compound is of one of Formulae XVII-a or XVII-b: 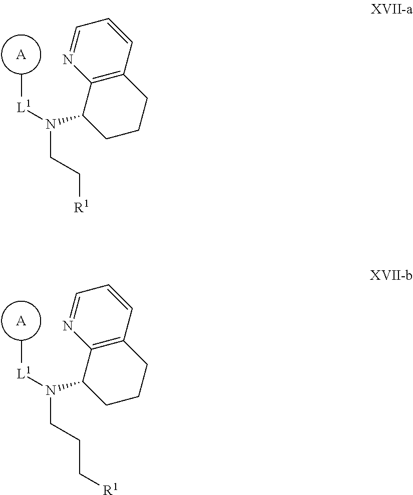
or a pharmaceutically acceptable salt thereof.

20. The compound of claim 1, wherein the compound is selected from one of the following: 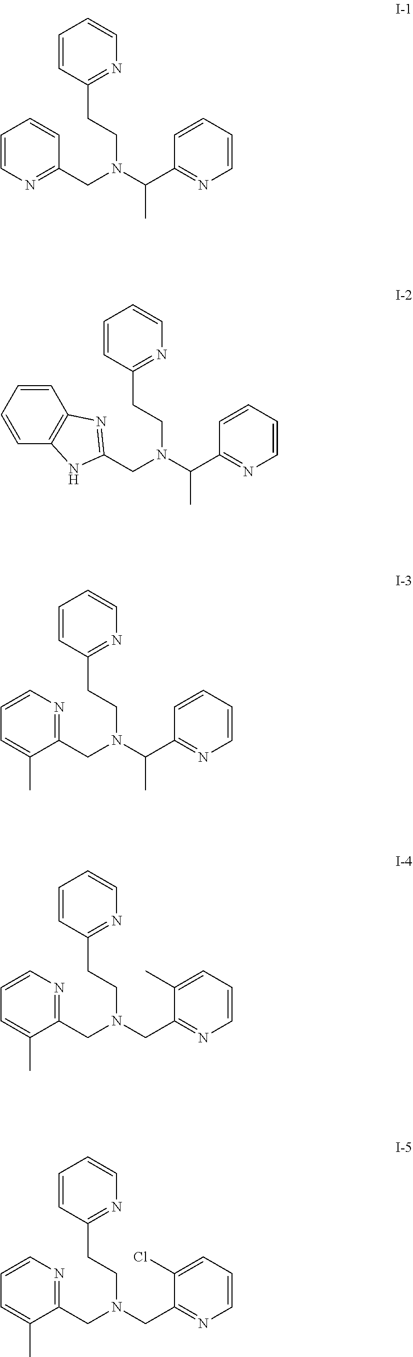 [SMILES: CC(c1ccccn1)N(CCc1ccccn1)Cc1ccccn1 ; CC(c1ccccn1)N(CCc1ccccn1)Cc1nc2ccccc2[nH]1 ; Cc1cccnc1CN(CCc1ccccn1)C(C)c1ccccn1 ; Cc1cccnc1CN(CCc1ccccn1)Cc1ncccc1C ; Cc1cccnc1CN(CCc1ccccn1)Cc1ncccc1Cl] 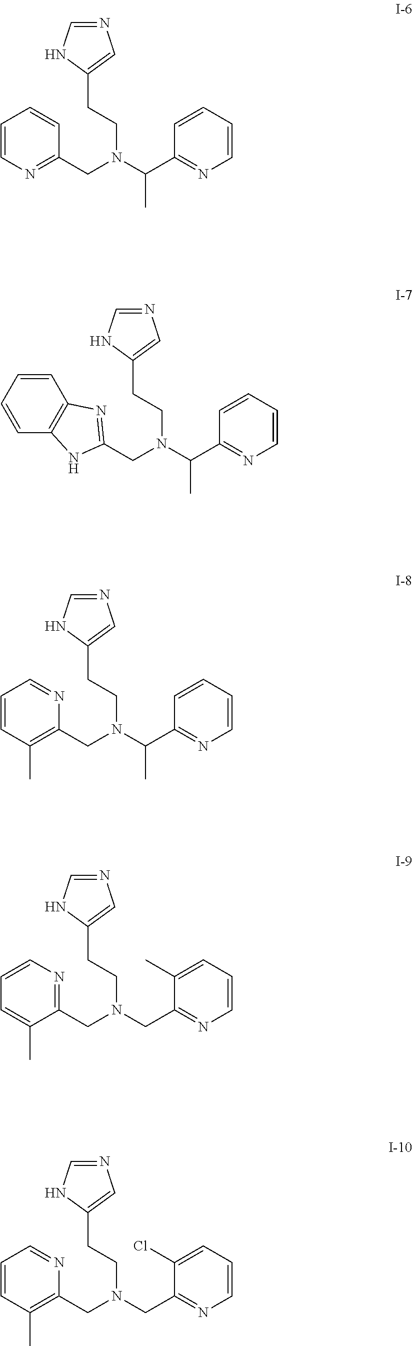 [SMILES: CC(c1ccccn1)N(CCc1cnc[nH]1)Cc1ccccn1 ; CC(c1ccccn1)N(CCc1cnc[nH]1)Cc1nc2ccccc2[nH]1 ; Cc1cccnc1CN(CCc1cnc[nH]1)C(C)c1ccccn1 ; Cc1cccnc1CN(CCc1cnc[nH]1)Cc1ncccc1C ; Cc1cccnc1CN(CCc1cnc[nH]1)Cc1ncccc1Cl] 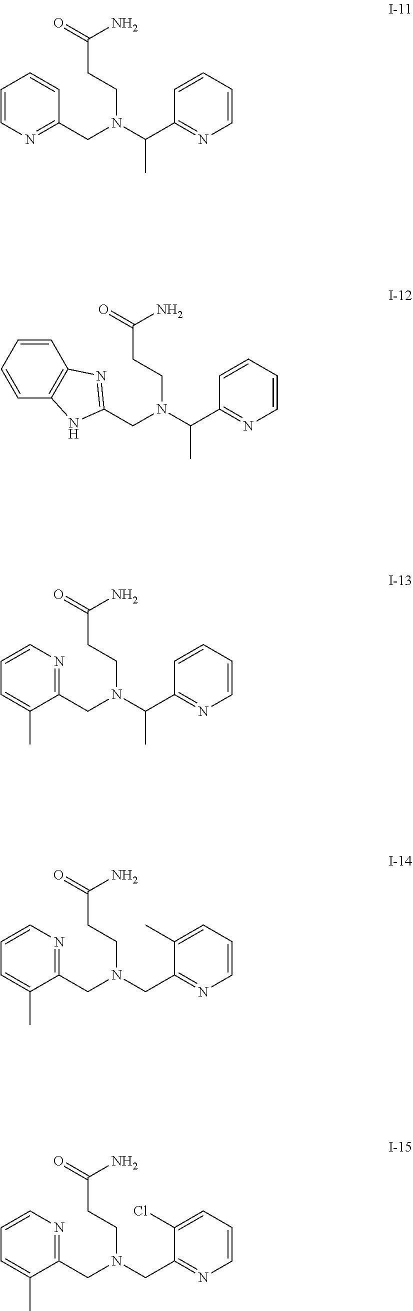 [SMILES: CC(c1ccccn1)N(CCC(N)=O)Cc1ccccn1 ; CC(c1ccccn1)N(CCC(N)=O)Cc1nc2ccccc2[nH]1 ; Cc1cccnc1CN(CCC(N)=O)C(C)c1ccccn1 ; Cc1cccnc1CN(CCC(N)=O)Cc1ncccc1C ; Cc1cccnc1CN(CCC(N)=O)Cc1ncccc1Cl] 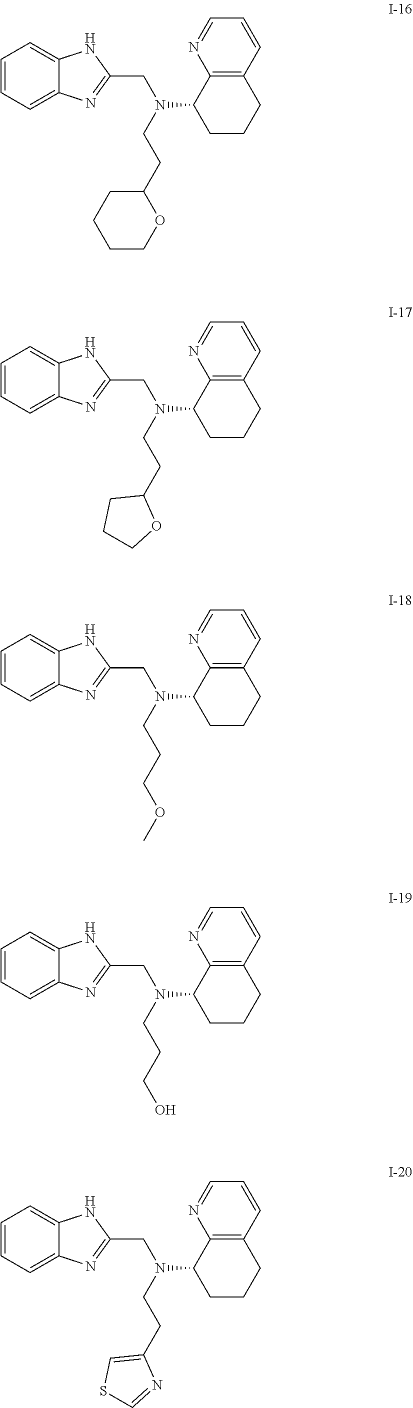 [SMILES: c1cnc2c(c1)CCC[C@@H]2N(CCC1CCCCO1)Cc1nc2ccccc2[nH]1 ; c1cnc2c(c1)CCC[C@@H]2N(CCC1CCCO1)Cc1nc2ccccc2[nH]1 ; COCCCN(Cc1nc2ccccc2[nH]1)[C@H]1CCCc2cccnc21 ; OCCCN(Cc1nc2ccccc2[nH]1)[C@H]1CCCc2cccnc21 ; c1cnc2c(c1)CCC[C@@H]2N(CCc1cscn1)Cc1nc2ccccc2[nH]1] 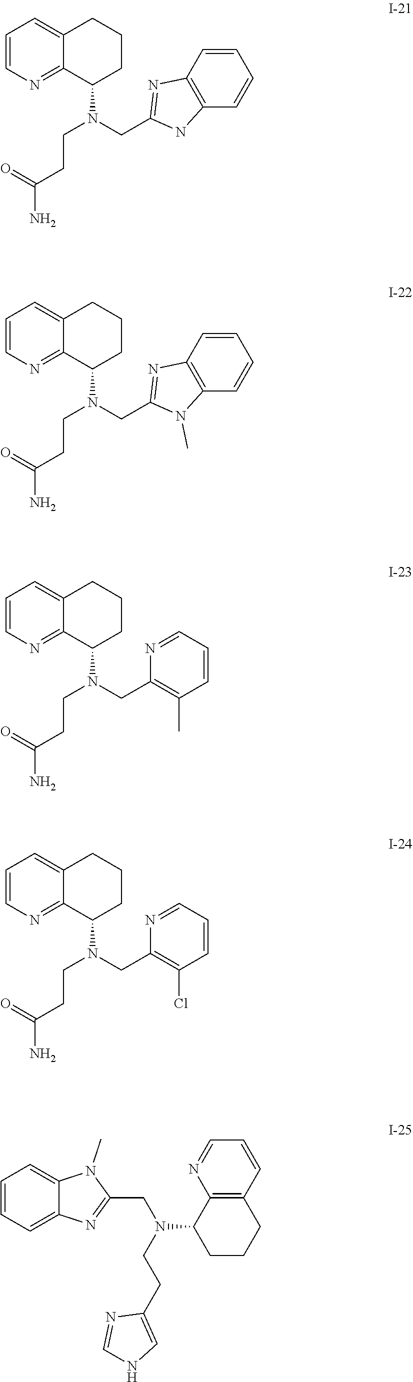 [SMILES: NC(=O)CCN(Cc1nc2ccccc2[nH]1)[C@H]1CCCc2cccnc21 ; Cn1c(CN(CCC(N)=O)[C@H]2CCCc3cccnc32)nc2ccccc21 ; Cc1cccnc1CN(CCC(N)=O)[C@H]1CCCc2cccnc21 ; NC(=O)CCN(Cc1ncccc1Cl)[C@H]1CCCc2cccnc21 ; Cn1c(CN(CCc2c[nH]cn2)[C@H]2CCCc3cccnc32)nc2ccccc21] 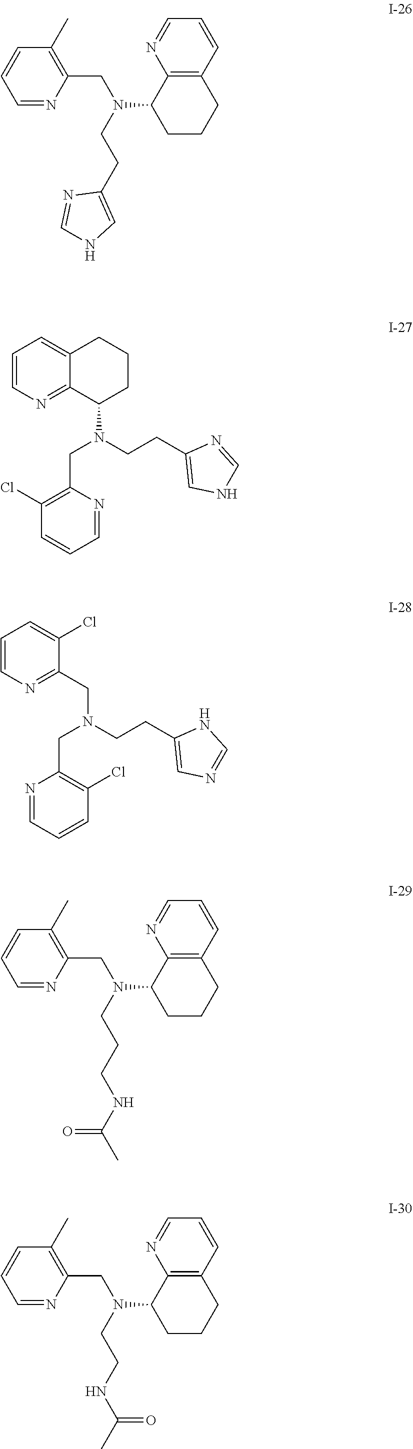 [SMILES: Cc1cccnc1CN(CCc1c[nH]cn1)[C@H]1CCCc2cccnc21 ; Clc1cccnc1CN(CCc1c[nH]cn1)[C@H]1CCCc2cccnc21 ; Clc1cccnc1CN(CCc1cnc[nH]1)Cc1ncccc1Cl ; CC(=O)NCCCN(Cc1ncccc1C)[C@H]1CCCc2cccnc21 ; CC(=O)NCCN(Cc1ncccc1C)[C@H]1CCCc2cccnc21] 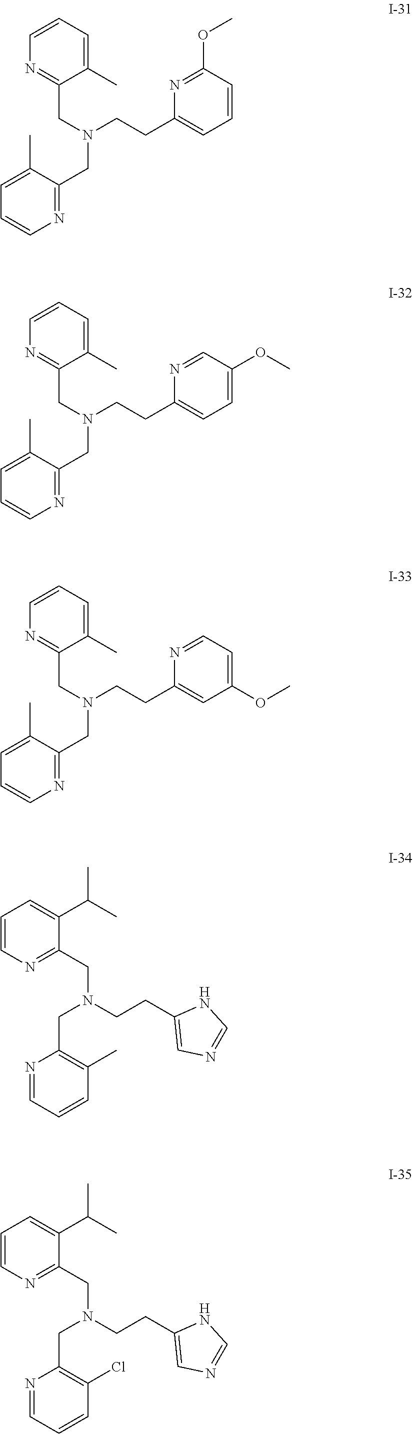 [SMILES: COc1cccc(CCN(Cc2ncccc2C)Cc2ncccc2C)n1 ; COc1ccc(CCN(Cc2ncccc2C)Cc2ncccc2C)nc1 ; COc1ccnc(CCN(Cc2ncccc2C)Cc2ncccc2C)c1 ; Cc1cccnc1CN(CCc1cnc[nH]1)Cc1ncccc1C(C)C ; CC(C)c1cccnc1CN(CCc1cnc[nH]1)Cc1ncccc1Cl] 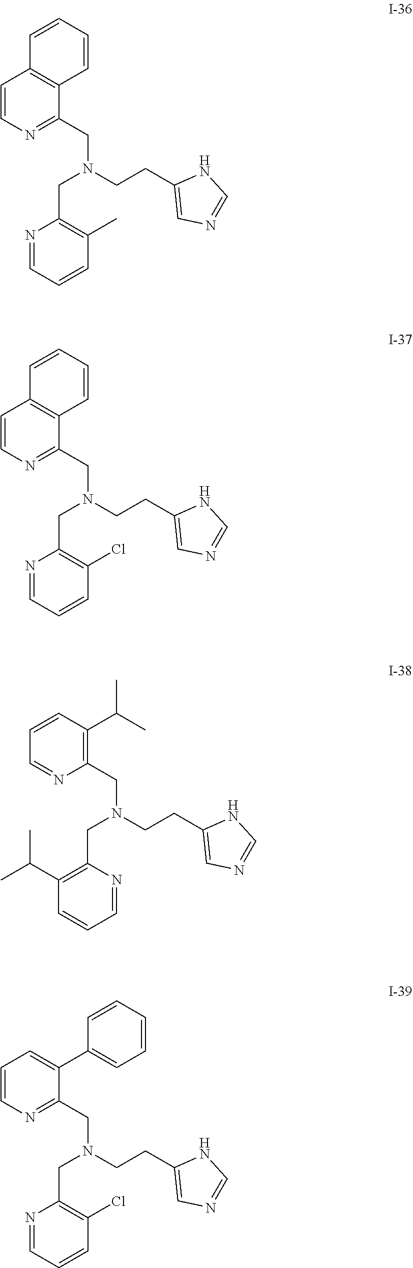 [SMILES: Cc1cccnc1CN(CCc1cnc[nH]1)Cc1nccc2ccccc12 ; Clc1cccnc1CN(CCc1cnc[nH]1)Cc1nccc2ccccc12 ; CC(C)c1cccnc1CN(CCc1cnc[nH]1)Cc1ncccc1C(C)C ; Clc1cccnc1CN(CCc1cnc[nH]1)Cc1ncccc1-c1ccccc1] 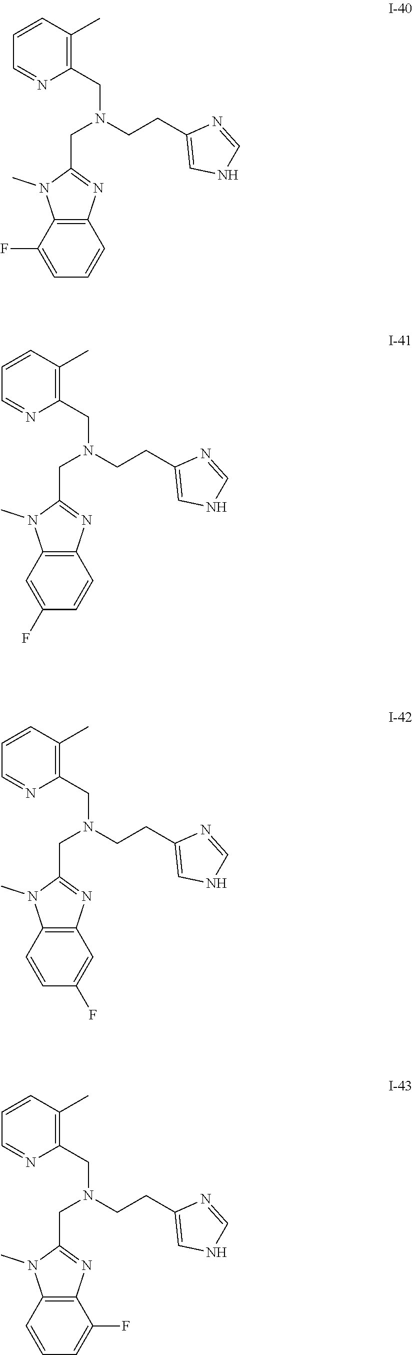 [SMILES: Cc1cccnc1CN(CCc1c[nH]cn1)Cc1nc2cccc(F)c2n1C ; Cc1cccnc1CN(CCc1c[nH]cn1)Cc1nc2ccc(F)cc2n1C ; Cc1cccnc1CN(CCc1c[nH]cn1)Cc1nc2cc(F)ccc2n1C ; Cc1cccnc1CN(CCc1c[nH]cn1)Cc1nc2c(F)cccc2n1C] 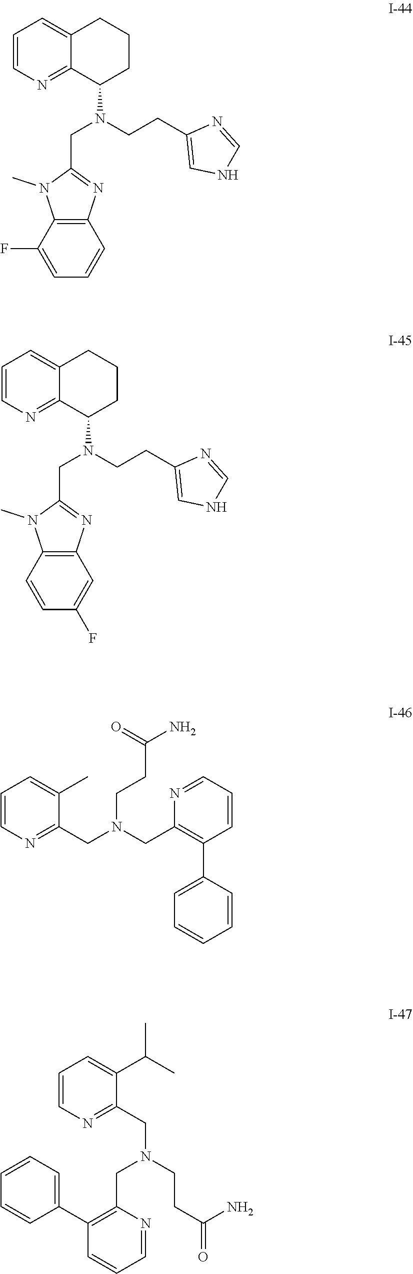 [SMILES: Cn1c(CN(CCc2c[nH]cn2)[C@H]2CCCc3cccnc32)nc2cccc(F)c21 ; Cn1c(CN(CCc2c[nH]cn2)[C@H]2CCCc3cccnc32)nc2cc(F)ccc21 ; Cc1cccnc1CN(CCC(N)=O)Cc1ncccc1-c1ccccc1 ; CC(C)c1cccnc1CN(CCC(N)=O)Cc1ncccc1-c1ccccc1] 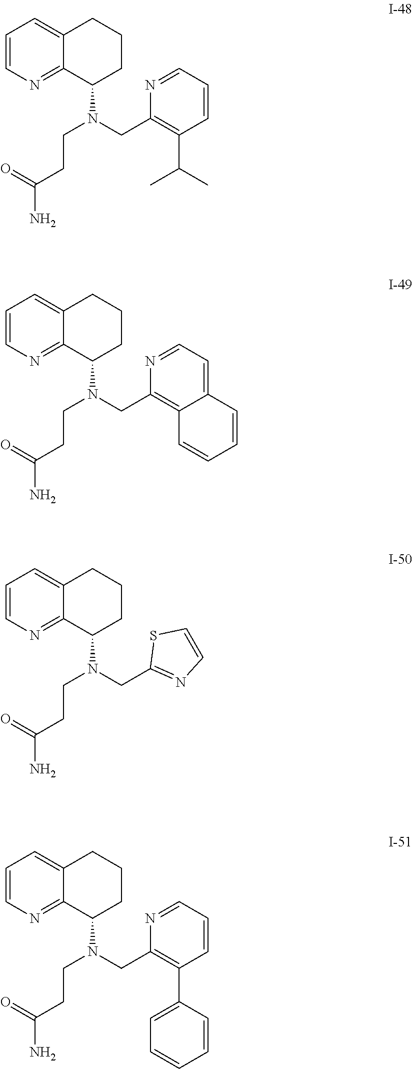 [SMILES: CC(C)c1cccnc1CN(CCC(N)=O)[C@H]1CCCc2cccnc21 ; NC(=O)CCN(Cc1nccc2ccccc12)[C@H]1CCCc2cccnc21 ; NC(=O)CCN(Cc1nccs1)[C@H]1CCCc2cccnc21 ; NC(=O)CCN(Cc1ncccc1-c1ccccc1)[C@H]1CCCc2cccnc21] 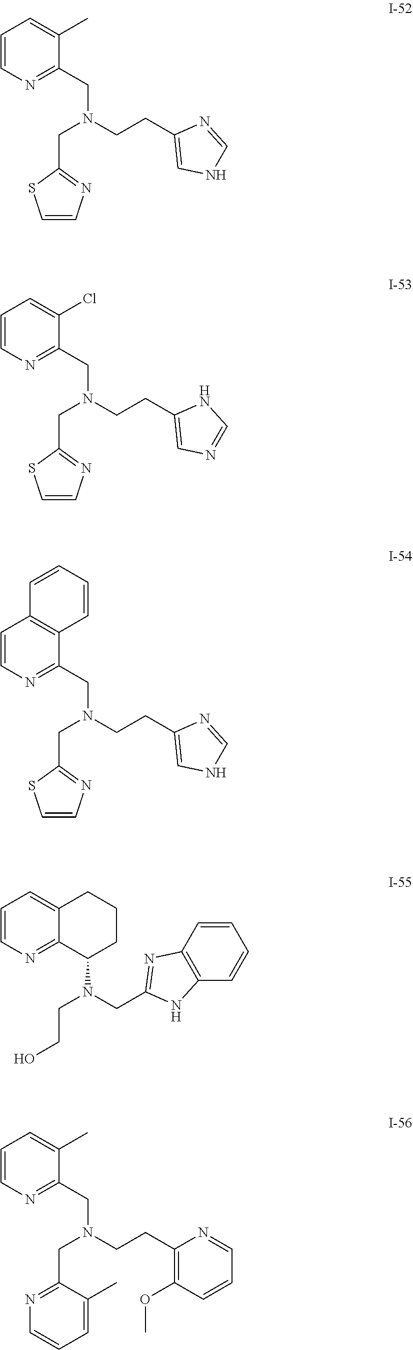 [SMILES: Cc1cccnc1CN(CCc1c[nH]cn1)Cc1nccs1 ; Clc1cccnc1CN(CCc1cnc[nH]1)Cc1nccs1 ; c1ccc2c(CN(CCc3c[nH]cn3)Cc3nccs3)nccc2c1 ; OCCN(Cc1nc2ccccc2[nH]1)[C@H]1CCCc2cccnc21 ; COc1cccnc1CCN(Cc1ncccc1C)Cc1ncccc1C] 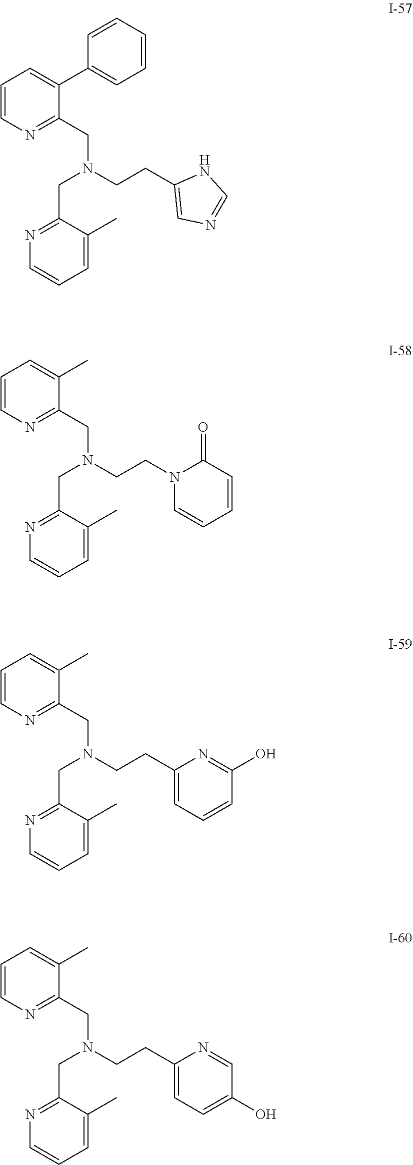 [SMILES: Cc1cccnc1CN(CCc1cnc[nH]1)Cc1ncccc1-c1ccccc1 ; Cc1cccnc1CN(CCn1ccccc1=O)Cc1ncccc1C ; Cc1cccnc1CN(CCc1cccc(O)n1)Cc1ncccc1C ; Cc1cccnc1CN(CCc1ccc(O)cn1)Cc1ncccc1C] 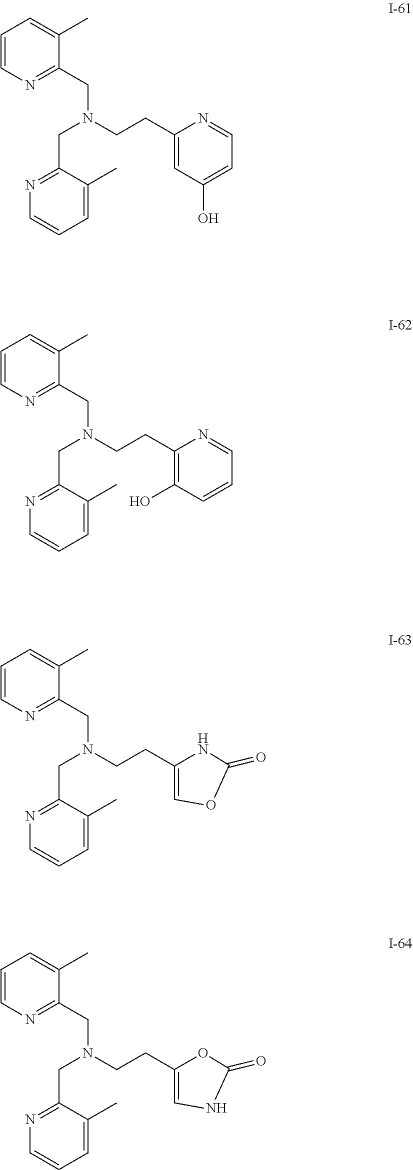 [SMILES: Cc1cccnc1CN(CCc1cc(O)ccn1)Cc1ncccc1C ; Cc1cccnc1CN(CCc1ncccc1O)Cc1ncccc1C ; Cc1cccnc1CN(CCc1coc(=O)[nH]1)Cc1ncccc1C ; Cc1cccnc1CN(CCc1c[nH]c(=O)o1)Cc1ncccc1C] 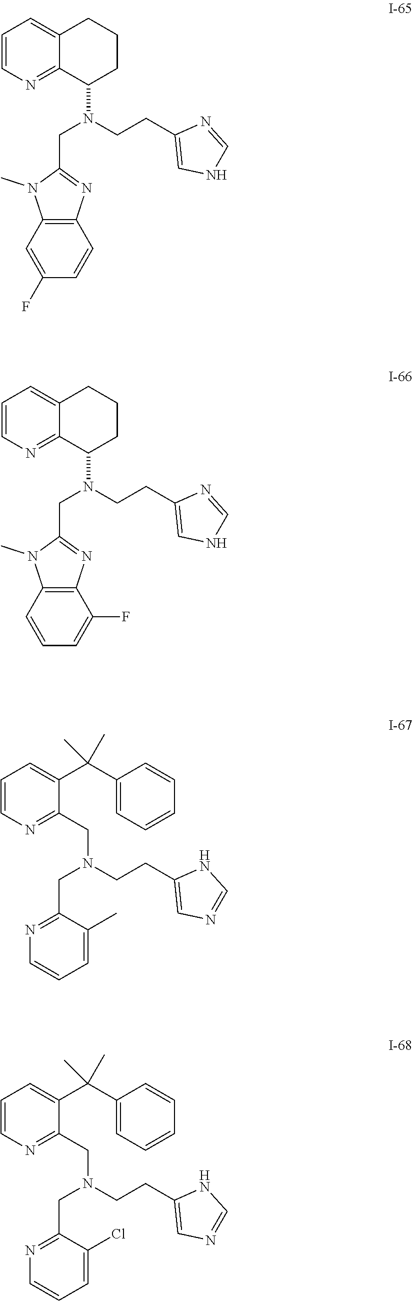 [SMILES: Cn1c(CN(CCc2c[nH]cn2)[C@H]2CCCc3cccnc32)nc2ccc(F)cc21 ; Cn1c(CN(CCc2c[nH]cn2)[C@H]2CCCc3cccnc32)nc2c(F)cccc21 ; Cc1cccnc1CN(CCc1cnc[nH]1)Cc1ncccc1C(C)(C)c1ccccc1 ; CC(C)(c1ccccc1)c1cccnc1CN(CCc1cnc[nH]1)Cc1ncccc1Cl] 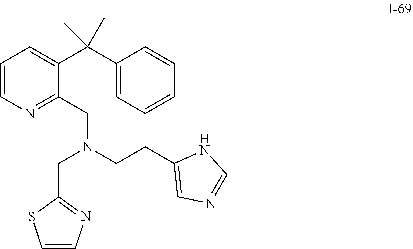 [SMILES: CC(C)(c1ccccc1)c1cccnc1CN(CCc1cnc[nH]1)Cc1nccs1]
or a pharmaceutically acceptable salt thereof.

21. A pharmaceutical composition comprising a compound of claim 1, or a pharmaceutically acceptable salt thereof, and a pharmaceutically acceptable excipient.

22. A method of treating a cancer selected from glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma, comprising administering to a patient in need thereof an effective amount of a compound of claim 1, or a pharmaceutically acceptable salt thereof.

23. A method of treating a cancer selected from acoustic neuroma, astrocytoma (Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma, comprising administering to a patient in need thereof an effective amount of a compound of any one of claim 1, or a pharmaceutically acceptable salt thereof.

24. A method of treating a cancer selected from brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor, comprising administering to a patient in need thereof an effective amount of a compound of claim 1, or a pharmaceutically acceptable salt thereof.

25. The method of claim 22, wherein the patient is an adult human.
